(19) United States
(12) Patent Application Publication
(10) Pub. No.: US 2022/0403136 A1
(43) Pub. Date: Dec. 22, 2022

(54) OPTICAL FILM FORMING COMPOSITION, OPTICAL FILM, AND DISPLAY DEVICE INCLUDING SAME

(71) Applicant: LG CHEM, LTD., Seoul (KR)

(72) Inventors: Bongjun KIM, Daejeon (KR); Sang Pil MOON, Daejeon (KR); Mun Kyu JOO, Daejeon (KR); Duy Hieu LE, Daejeon (KR); Yeon Keun LEE, Daejeon (KR); Hoyong LEE, Daejeon (KR)

(73) Assignee: LG CHEM, LTD., Seoul (KR)

(21) Appl. No.: 17/773,334

(22) PCT Filed: Mar. 4, 2021
(86) PCT No.: PCT/KR2021/002683

§ 371 Date: Apr. 29, 2022

(30) Foreign Application Priority Data
Mar. 4, 2020 (KR) 10-2020-0027228

(51) Int. Cl. C08K 5/00; C07F 15/00; C07F 15/04; C07F 15/06; C07F 3/06; G02B 1/04; C08J 5/18; H01L 51/52

(52) U.S. Cl. CPC C08K 5/0041; C07F 15/0066; C07F 15/045; C07F 15/065; C07F 3/06; G02B 1/04; C08J 5/18; H01L 51/52

(57) ABSTRACT

The present specification relates to a composition for forming an optical film comprising a compound represented by Chemical Formula 1 and a binder resin, an optical film, and a display device comprising the same.

This application is a 35 U.S.C. 371 National Phase Entry Application from PCT/KR2021/002683, filed on Mar. 4, 2021, which claims priority to and the benefits of Korean Patent Application No. 10-2020-0027228, filed with the Korean Intellectual Property Office on Mar. 4, 2020, the entire contents of which are incorporated herein by reference.

### TECHNICAL FIELD

The present specification relates to a composition for forming an optical film, an optical film, and a display device comprising the same.

### BACKGROUND OF THE INVENTION

An electrode in an organic light emitting diode (OLED) is made of a metal element, which causes a problem of significantly reducing a contrast ratio of a display due to high external light reflectance. In view of the above, a circular polarizing plate is laminated to suppress external light reflection, however, this method causes disadvantages of increasing material costs and reducing flexibility as well as failing to flexibly adjust color tones of light emitting colors.

With the purpose of replacing this, a low-cost optical film for anti-reflection introducing a dye and a pigment to an adhesive film has been developed. Currently, such an optical film is inferior in viewing angle properties compared to a circular polarizing plate.

### BRIEF SUMMARY OF THE INVENTION

The present specification is directed to providing a composition for forming an optical film, an optical film, and a display device including the same.

One embodiment of the present specification provides a composition for forming an optical film, the composition comprising a compound represented by the following Chemical Formula 1, and a binder resin.

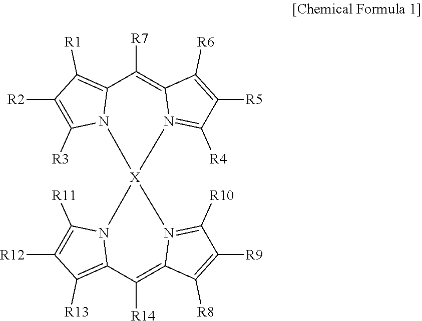

In Chemical Formula 1,

X is Zn; Co; Ni or Pd,

R1 to R6 and R8 to R13 are the same as or different from each other, and each independently hydrogen; deuterium; a halogen group; a nitrile group; a nitro group; —OC(═O)R; —(C═O)NR′R″; —(C═O)OR′″; —(C═O)Rx; a substituted or unsubstituted alkyl group; a substituted or unsubstituted cycloalkyl group; a substituted or unsubstituted aryl group; a substituted or unsubstituted alkoxy group; a substituted or unsubstituted aryloxy group; a substituted or unsubstituted heteroaryloxy group; a substituted or unsubstituted alkylthio group; a substituted or unsubstituted arylthio group; a substituted or unsubstituted heteroarylthio group; or a substituted or unsubstituted heteroaryl group,

R7 and R14 are the same as or different from each other, and each independently hydrogen; a substituted or unsubstituted alkyl group; a substituted or unsubstituted aryl group; a substituted or unsubstituted heteroaryl group; a substituted or unsubstituted aryloxy group; a substituted or unsubstituted arylthio group; a substituted or unsubstituted heteroarylthio group; or a substituted or unsubstituted heteroaryloxy group,

R, R′, R″, R′″ and Rx are the same as or different from each other, and each independently hydrogen; a substituted or unsubstituted alkyl group; a substituted or unsubstituted cycloalkyl group; a substituted or unsubstituted heterocyclic group; or a substituted or unsubstituted aryl group, or R′ and R″ bond to each other to form a ring, and

when R7 and R14 are hydrogen; a substituted or unsubstituted alkyl group; a substituted or unsubstituted aryl group; or a substituted or unsubstituted heteroaryl group and R2, R5, R9 and R12 are —C(═O)OR′″, R′″ is a substituted or unsubstituted aryl group; a substituted or unsubstituted cycloalkyl group; a substituted alkyl group; or a branched unsubstituted alkyl group having 3 to 30 carbon atoms.

Another embodiment of the present specification provides an optical film comprising the composition for forming an optical film or a cured material thereof.

Another embodiment of the present specification provides an adhesive optical filter comprising the optical film; and a surface treatment layer.

Another embodiment of the present specification provides a display device comprising the optical film.

### Advantageous Effects

An optical film comprising a composition for forming an optical film according to one embodiment of the present specification or a cured material thereof effectively absorbs a wavelength of 490 nm to 590 nm, and has excellent reliability that cannot be obtained in general-purpose dyes.

### BRIEF DESCRIPTION OF DRAWINGS

FIG. 1 to FIG. 4 are diagrams showing wavelength-dependent absorption spectra of optical films according to examples.

FIG. 5 is a diagram showing wavelength-dependent absorption spectra of optical films according to comparative examples.

FIG. 6 illustrates a structure of an adhesive film including a release layer provided on one surface of the adhesive film according to one embodiment of the present specification.

FIG. 7 illustrates a structure of an adhesive optical filter according to one embodiment of the present specification.

FIG. 8 illustrates a structure of an OLED device, one example of a display device according to one embodiment of the present specification.

FIG. 9 illustrates a structure of an OLED panel according to one embodiment of the present specification.

FIG. 10 illustrates an OLED device having a bottom emission structure (a) and an OLED device having a top emission structure according to the present specification (b).

FIG. 11 illustrates examples of a structure of an OLED panel provided with a color filter-formed substrate according to the present specification.

FIG. 12 illustrates examples of a structure of a white OLED panel provided with a substrate having a white pixel-including color filter formed therein according to the present specification.

### REFERENCE NUMERAL

1: Surface Treatment Layer
2: Binder Resin Film
3: Adhesive Film
4: Release Layer
10: Adhesive Optical Filter
11: Substrate
12: Lower Electrode
13: Organic Material Layer
14: Upper Electrode
15: Encapsulation Substrate
16: Color Filter-Formed Substrate
17: Substrate Having White Pixel-Including Color Filter Formed Therein
20: OLED Panel
30: OLED Device

### DETAILED DESCRIPTION OF THE INVENTION

One embodiment of the present specification provides a composition for forming an optical film, the composition comprising a compound represented by the following Chemical Formula 1, and a binder resin.

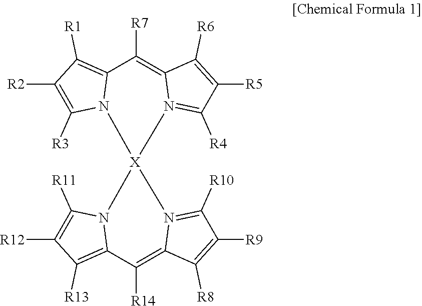

In Chemical Formula 1,

X is Zn; Co; Ni or Pd,

R1 to R6 and R8 to R13 are the same as or different from each other, and each independently hydrogen; deuterium; a halogen group; a nitrile group; a nitro group; —OC(═O)R; —(C═O)NR′R″; —(C═O)OR′″; —(C═O)Rx; a substituted or unsubstituted alkyl group; a substituted or unsubstituted cycloalkyl group; a substituted or unsubstituted aryl group; a substituted or unsubstituted alkoxy group; a substituted or unsubstituted aryloxy group; a substituted or unsubstituted heteroaryloxy group; a substituted or unsubstituted alkylthio group; a substituted or unsubstituted arylthio group; a substituted or unsubstituted heteroarylthio group; or a substituted or unsubstituted heteroaryl group,

R7 and R14 are the same as or different from each other, and each independently hydrogen; a substituted or unsubstituted alkyl group; a substituted or unsubstituted aryl group; a substituted or unsubstituted heteroaryl group; a substituted or unsubstituted aryloxy group; a substituted or unsubstituted arylthio group; a substituted or unsubstituted heteroarylthio group; or a substituted or unsubstituted heteroaryloxy group,

R, R′, R″, R′″ and Rx are the same as or different from each other, and each independently hydrogen; a substituted or unsubstituted alkyl group; a substituted or unsubstituted cycloalkyl group; a substituted or unsubstituted heterocyclic group; or a substituted or unsubstituted aryl group, or R′ and R″ bond to each other to form a ring, and

when R7 and R14 are hydrogen; a substituted or unsubstituted alkyl group; a substituted or unsubstituted aryl group; or a substituted or unsubstituted heteroaryl group and R2, R5, R9 and R12 are —C(═O)OR′″, R′″ is a substituted or unsubstituted aryl group; a substituted or unsubstituted cycloalkyl group; a substituted alkyl group; or a branched unsubstituted alkyl group having 3 to 30 carbon atoms.

By including the compound represented by Chemical Formula 1, the composition for forming an optical film according to one embodiment of the present specification is effective in controlling a transmission color and a reflection color of a display device in a direction that a designer desires due to an operating principle of absorbing a light source with a wavelength of 450 nm to 600 nm.

Specifically, when using the composition for forming an optical film according to one embodiment of the present specification in an OLED device, external light reflection may be efficiently suppressed without using a circular polarizing plate. Not using a circular polarizing plate in an OLED device has advantages of saving material costs, and properly maintaining flexibility of the OLED device.

In addition, by the present specification providing a black color optical film incorporating an organic dye capable of absorbing visible light, an OLED device including the same may suppress high panel reflectance.

In the present specification, the term “substituted or unsubstituted” means being substituted with one, two or more substituents selected from the group consisting of a halogen group; an alkyl group; a cycloalkyl group; an aryl group; an alkoxy group; an aryloxy group; an alkylthio group; an arylthio group; a heteroaryloxy group; a heteroarylthio group; and a heterocyclic group, or being substituted with a substituent linking two or more substituents among the substituents illustrated above, or having no substituents.

In the present specification, the halogen group may be fluorine, chlorine, bromine or iodine.

In the present specification, the alkyl group may be linear or branched, and although not particularly limited thereto, the number of carbon atoms is preferably from 1 to 30. Specific examples thereof may include methyl, ethyl, propyl, isopropyl, butyl, heptyl and the like, but are not limited thereto.

In the present specification, the cycloalkyl group is not particularly limited, but preferably has 3 to 30 carbon atoms, and specific examples thereof may include a cyclopropyl group, a cyclobutyl group, a cyclopentyl group, a cycloheptyl group, a cyclooctyl group and the like, but are not limited thereto.

In the present specification, the aryl group may be monocyclic or polycyclic.

When the aryl group is a monocyclic aryl group, the number of carbon atoms is not particularly limited, but is preferably from 6 to 30. Specific examples of the monocyclic aryl group may include a phenyl group, a biphenyl group, a terphenyl group and the like, but are not limited thereto.

When the aryl group is a polycyclic aryl group, the number of carbon atoms is not particularly limited, but is preferably from 10 to 30. Specific examples of the polycyclic aryl group may include a naphthyl group, an anthracenyl group, a phenanthryl group, a pyrenyl group, a perylenyl group, a chrysenyl group, a fluorenyl group and the like, but are not limited thereto.

In the present specification, the alkoxy group may be linear, branched or cyclic. The number of carbon atoms of the alkoxy group is not particularly limited, but is preferably from 1 to 20. Specific examples thereof may include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the aryl group in the aryloxy group and the arylthio group is the same as the examples of the aryl group described above. Specific examples of the aryloxy group may include phenoxy, p-tolyloxy, m-tolyloxy, 3,5-dimethyl-phenoxy, 2,4,6-trimethylphenoxy, p-tert-butylphenoxy, 3-biphenyloxy, 4-biphenyloxy, 1-naphthyloxy, 2-naphthyloxy, 4-methyl-1-naphthyloxy, 5-methyl-2-naphthyloxy, 1-anthryloxy, 2-anthryloxy, 9-anthryloxy, 1-phenanthryloxy, 3-phenanthryloxy, 9-phenanthryloxy and the like, and specific examples of the arylthio group may include a phenylthio group, a 2-methylphenylthio group, a 4-tert-butylphenylthio group and the like, however, the aryloxy group and the arylthiol group are not limited thereto.

In the present specification, the heterocyclic group is a group including one or more atoms that are not carbon, that is, heteroatoms, and specifically, the heteroatom may include one or more atoms selected from the group consisting of O, N, Se, S and the like. The number of carbon atoms is not particularly limited, but is preferably from 2 to 30, and the heterocyclic group may be monocyclic or polycyclic. Examples of the heterocyclic group may include a thiophene group, a pyridyl group, a pyrimidyl group, a triazinyl group, a benzothiophene group, a dibenzothiophene group, a benzofuranyl group, a dibenzofuranyl group and the like, but are not limited thereto.

In the present specification, the heteroaryl group means an aromatic group among the heterocyclic group.

In the present specification, the alkyl group in the alkylthio group is the same as the examples of the alkyl group described above. Specific examples of the alkylthio group may include a methylthio group, an ethylthio group, a tert-butylthio group, a hexylthio group, an octylthio group and the like, but are not limited thereto.

In the present specification, the aryl group in the arylthio group is the same as the examples of the aryl group described above.

In one embodiment of the present specification, the coumarin group and the chromone group are respectively represented by

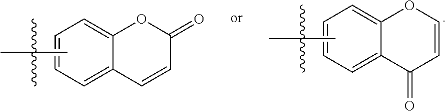

In the present specification, the heteroaryl group in the heteroaryloxy group and the heteroarylthio group is the same as the examples of the heteroaryl group described above.

In one embodiment of the present specification, X is Zn; Co; Ni or Pd.

In one embodiment of the present specification, X is Zn.

In one embodiment of the present specification, X is Co.

In one embodiment of the present specification, X is Pd.

In one embodiment of the present specification, X is Ni.

In one embodiment of the present specification, R1 to R6 and R8 to R13 are the same as or different from each other, and each independently hydrogen; deuterium; a halogen group; a nitrile group; a nitro group; —OC(═O)R; —(C═O)NR′R″; —(C═O)OR′″; —(C═O)Rx; a substituted or unsubstituted alkyl group; a substituted or unsubstituted cycloalkyl group; a substituted or unsubstituted aryl group; a substituted or unsubstituted alkoxy group; a substituted or unsubstituted aryloxy group; a substituted or unsubstituted heteroaryloxy group; a substituted or unsubstituted alkylthio group; a substituted or unsubstituted arylthio group; a substituted or unsubstituted heteroarylthio group; or a substituted or unsubstituted heteroaryl group.

In one embodiment of the present specification, R1 to R6 and R8 to R13 are the same as or different from each other, and each independently hydrogen; deuterium; a halogen group; a nitrile group; a nitro group; —OC(═O)R; —(C═O)NR′R″; —(C═O)OR′″; —(C═O)Rx; a substituted or unsubstituted alkyl group having 1 to 30 carbon atoms; a substituted or unsubstituted cycloalkyl group having 3 to 30 carbon atoms; a substituted or unsubstituted aryl group having 6 to 30 carbon atoms; a substituted or unsubstituted alkoxy group having 1 to 30 carbon atoms; a substituted or unsubstituted aryloxy group having 6 to 30 carbon atoms; a substituted or unsubstituted heteroaryloxy group having 2 to 30 carbon atoms; a substituted or unsubstituted alkylthio group having 1 to 30 carbon atoms; a substituted or unsubstituted arylthio group having 6 to 30 carbon atoms; a substituted or unsubstituted heteroarylthio group having 2 to 30 carbon atoms; or a substituted or unsubstituted heteroaryl group having 2 to 30 carbon atoms.

In one embodiment of the present specification, R1 to R6 and R8 to R13 are the same as or different from each other, and each independently hydrogen; deuterium; a halogen group; a nitrile group; a nitro group; —OC(═O)R; —(C═O)NR′R″; —(C═O)OR′″; —(C═O)Rx; a substituted or unsubstituted alkyl group having 1 to 20 carbon atoms; a substituted or unsubstituted cycloalkyl group having 3 to 20 carbon atoms; a substituted or unsubstituted aryl group having 6 to 20 carbon atoms; a substituted or unsubstituted alkoxy group having 1 to 20 carbon atoms; a substituted or unsubstituted aryloxy group having 6 to 20 carbon atoms; a substituted or unsubstituted heteroaryloxy group having 2 to 20 carbon atoms; a substituted or unsubstituted alkylthio group having 1 to 20 carbon atoms; a substituted or unsubstituted arylthio group having 6 to 20 carbon atoms; a substituted or unsubstituted heteroarylthio group having 2 to 20 carbon atoms; or a substituted or unsubstituted heteroaryl group having 2 to 20 carbon atoms.

In one embodiment of the present specification, R1 to R6 and R8 to R13 are the same as or different from each other, and each independently hydrogen; deuterium; a halogen group; a nitrile group; a nitro group; —OC(═O)R; —(C═O)NR′R″; —(C═O)OR′″; —(C═O)Rx; a substituted or unsubstituted alkyl group having 1 to 10 carbon atoms; a substituted or unsubstituted cycloalkyl group having 3 to 10 carbon atoms; a substituted or unsubstituted aryl group having 6 to 12 carbon atoms; a substituted or unsubstituted alkoxy group having 1 to 10 carbon atoms; a substituted or unsubstituted aryloxy group having 6 to 12 carbon atoms; a substituted or unsubstituted heteroaryloxy group having 2 to 10 carbon atoms; a substituted or unsubstituted alkylthio group having 1 to 10 carbon atoms; a substituted or unsubstituted arylthio group having 6 to 12 carbon atoms; a substituted or unsubstituted heteroarylthio group having 2 to 12 carbon atoms; or a substituted or unsubstituted heteroaryl group having 2 to 12 carbon atoms.

In one embodiment of the present specification, R7 and R14 are the same as or different from each other, and each independently hydrogen; a substituted or unsubstituted alkyl group; a substituted or unsubstituted aryl group; a substituted or unsubstituted heteroaryl group; a substituted or unsubstituted aryloxy group; a substituted or unsubstituted arylthio group; a substituted or unsubstituted heteroarylthio group; or a substituted or unsubstituted heteroaryloxy group.

In one embodiment of the present specification, R7 and R14 are the same as or different from each other, and each independently hydrogen; a substituted or unsubstituted alkyl group having 1 to 30 carbon atoms; a substituted or unsubstituted aryl group having 6 to 30 carbon atoms; a substituted or unsubstituted heteroaryl group having 2 to 30 carbon atoms; a substituted or unsubstituted aryloxy group having 6 to 30 carbon atoms; a substituted or unsubstituted arylthio group having 6 to 30 carbon atoms; a substituted or unsubstituted heteroarylthio group having 2 to 30 carbon atoms; or a substituted or unsubstituted heteroaryloxy group having 2 to 30 carbon atoms.

In one embodiment of the present specification, R7 and R14 are the same as or different from each other, and each independently hydrogen; a substituted or unsubstituted alkyl group having 1 to 20 carbon atoms; a substituted or unsubstituted aryl group having 6 to 20 carbon atoms; a substituted or unsubstituted heteroaryl group having 2 to 20 carbon atoms; a substituted or unsubstituted aryloxy group having 6 to 20 carbon atoms; a substituted or unsubstituted arylthio group having 6 to 20 carbon atoms; a substituted or unsubstituted heteroarylthio group having 2 to 20 carbon atoms; or a substituted or unsubstituted heteroaryloxy group having 2 to 20 carbon atoms.

In one embodiment of the present specification, R7 and R14 are the same as or different from each other, and each independently hydrogen; a substituted or unsubstituted alkyl group having 1 to 10 carbon atoms; a substituted or unsubstituted aryl group having 6 to 12 carbon atoms; a substituted or unsubstituted heteroaryl group having 2 to 10 carbon atoms; a substituted or unsubstituted aryloxy group having 6 to 12 carbon atoms; a substituted or unsubstituted arylthio group having 6 to 12 carbon atoms; a substituted or unsubstituted heteroarylthio group having 2 to 10 carbon atoms; or a substituted or unsubstituted heteroaryloxy group having 2 to 10 carbon atoms.

In one embodiment of the present specification, R, R′, R″, R′″ and Rx are the same as or different from each other, and each independently hydrogen; a substituted or unsubstituted alkyl group; a substituted or unsubstituted cycloalkyl group; a substituted or unsubstituted heterocyclic group; or a substituted or unsubstituted aryl group, or R′ and R″ bond to each other to form a ring. In one embodiment of the present specification, R, R′, R″, R′″ and Rx are the same as or different from each other, and each independently hydrogen; a substituted or unsubstituted alkyl group having 1 to 30 carbon atoms; a substituted or unsubstituted cycloalkyl group having 3 to 30 carbon atoms; a substituted or unsubstituted heteroaryl group having 2 to 30 carbon atoms; or a substituted or unsubstituted aryl group having 6 to 30 carbon atoms, or R′ and R″ bond to each other to form an O-including heteroring.

In one embodiment of the present specification, R, R′, R″, R′″ and Rx are the same as or different from each other, and each independently hydrogen; a substituted or unsubstituted alkyl group having 1 to 20 carbon atoms; a substituted or unsubstituted cycloalkyl group having 3 to 20 carbon atoms; a substituted or unsubstituted heteroaryl group having 2 to 20 carbon atoms; or a substituted or unsubstituted aryl group having 6 to 20 carbon atoms, or R′ and R″ bond to each other to form an O-including heteroring.

In one embodiment of the present specification, R, R′, R″, R′″ and Rx are the same as or different from each other, and each independently hydrogen; a substituted or unsubstituted alkyl group having 1 to 10 carbon atoms; a substituted or unsubstituted cycloalkyl group having 3 to 10 carbon atoms; a substituted or unsubstituted heteroaryl group having 2 to 10 carbon atoms; or a substituted or unsubstituted aryl group having 6 to 12 carbon atoms, or R′ and R″ bond to each other to form an O-including heteroring.

In one embodiment of the present specification, R1 to R6 and R8 to R13 are the same as or different from each other, and each independently hydrogen; deuterium; fluorine; a nitrile group; a nitro group; —(C═O)NR′R″; —(C═O)OR′″; —(C═O)Rx; a substituted or unsubstituted methyl group; a substituted or unsubstituted isopropyl group; a substituted or unsubstituted cyclopentyl group; a substituted or unsubstituted cyclohexyl group; a substituted or unsubstituted phenyl group; or a substituted or unsubstituted dibenzofuranyl group.

In one embodiment of the present specification, R1 to R6 and R8 to R13 are the same as or different from each other, and each independently hydrogen; fluorine; a nitrile group; a nitro group; —(C═O)NR′R″; —(C═O)OR′″; —(C═O)Rx; a methyl group; a trifluoromethyl group; an isopropyl group; a cyclopentyl group substituted with a methyl group; a cyclohexyl group unsubstituted or substituted with a methyl group or an isopropyl group; a phenyl group substituted with a trifluoromethyl group; or a dibenzofuranyl group.

In one embodiment of the present specification, R1, R6, R8 and R13 are the same as or different from each other, and each independently hydrogen; deuterium; a substituted or unsubstituted alkyl group; or a substituted or unsubstituted cycloalkyl group.

In one embodiment of the present specification, R1, R6, R8 and R13 are the same as or different from each other, and each independently hydrogen; deuterium; a substituted or unsubstituted alkyl group having 1 to 30 carbon atoms; or a substituted or unsubstituted cycloalkyl group having 3 to 30 carbon atoms.

In one embodiment of the present specification, R1, R6, R8 and R13 are the same as or different from each other, and each independently hydrogen; deuterium; a substituted or unsubstituted alkyl group having 1 to 20 carbon atoms; or a substituted or unsubstituted cycloalkyl group having 3 to 20 carbon atoms.

In one embodiment of the present specification, R1, R6, R8 and R13 are the same as or different from each other, and each independently hydrogen; deuterium; a substituted or unsubstituted alkyl group having 1 to 10 carbon atoms; or a substituted or unsubstituted cycloalkyl group having 3 to 10 carbon atoms.

In one embodiment of the present specification, R1, R6, R8 and R13 are the same as or different from each other, and each independently hydrogen; a substituted or unsubstituted methyl group; or a substituted or unsubstituted cyclohexyl group.

In one embodiment of the present specification, R1, R6, R8 and R13 are the same as or different from each other, and each independently hydrogen; a methyl group; or a cyclohexyl group unsubstituted or substituted with a methyl group or an isopropyl group.

In one embodiment of the present specification, at least one of R2, R5, R9 and R12 is a halogen group; a nitrile group; a nitro group; —(C═O)NR′R″; —(C═O)OR′″; —(C═O)Rx; a substituted or unsubstituted aryl group; a substituted or unsubstituted heteroaryl group; or an alkyl group substituted with fluorine.

In one embodiment of the present specification, at least one of R2, R5, R9 and R12 is chlorine; fluorine; bromine; iodine; a nitrile group; a nitro group; —(C═O)NR′R″; —(C═O)OR′″; —(C═O)Rx; a substituted or unsubstituted aryl group having 6 to 30 carbon atoms; a substituted or unsubstituted heteroaryl group having 3 to 30 carbon atoms; or an alkyl group having 1 to 30 carbon atoms substituted with fluorine.

In one embodiment of the present specification, at least one of R2, R5, R9 and R12 is chlorine; fluorine; bromine; iodine; a nitrile group; a nitro group; —(C═O)NR′R″; —(C═O)OR′″; —(C═O)Rx; a substituted or unsubstituted aryl group having 6 to 20 carbon atoms; a substituted or unsubstituted heteroaryl group having 3 to 20 carbon atoms; or an alkyl group having 1 to 20 carbon atoms substituted with fluorine.

In one embodiment of the present specification, at least one of R2, R5, R9 and R12 is chlorine; fluorine; bromine; iodine; a nitrile group; a nitro group; —(C═O)NR′R″; —(C═O)OR′″; —(C═O)Rx; a substituted or unsubstituted aryl group having 6 to 12 carbon atoms; a substituted or unsubstituted heteroaryl group having 3 to 12 carbon atoms; or an alkyl group having 1 to 10 carbon atoms substituted with fluorine.

In one embodiment of the present specification, at least one of R2, R5, R9 and R12 is chlorine; fluorine; bromine; iodine; a nitrile group; a nitro group; —(C═O)NR′R″; —(C═O)OR′″; —(C═O)Rx; a phenyl group substituted with a trifluoromethyl group; or a methyl group substituted with fluorine.

In one embodiment of the present specification, at least one of R2, R5, R9 and R12 is chlorine; fluorine; bromine; iodine; a nitrile group; a phenyl group substituted with a trifluoromethyl group; —(C═O)OR′″; or —CONR′R″.

In one embodiment of the present specification, R, R′, R″, R′″ and Rx are the same as or different from each other, and each independently hydrogen; a substituted or unsubstituted methyl group; a substituted or unsubstituted ethyl group; a substituted or unsubstituted cyclohexyl group; or a substituted or unsubstituted phenyl group, or R′ and R″ bond to each other to form a morpholinyl group.

In one embodiment of the present specification, R, R′, R″, R′″ and Rx are the same as or different from each other, and each independently hydrogen; a methyl group; an ethyl group; a methyl group substituted with an alkoxy group; a methyl group substituted with a phenyl group substituted with a nitro group; a phenyl group; a phenyl group substituted with a nitro group; a phenyl group substituted with a tert-butyl group; or a phenyl group substituted with a nitrile group, or R′ and R″ bond to each other to form a morpholinyl group.

In one embodiment of the present specification, R′″ is a methyl group; an ethyl group; a methyl group substituted with an alkoxy group; a methyl group substituted with a phenyl group substituted with a nitro group; a phenyl group substituted with a nitro group; a phenyl group substituted with a tert-butyl group; or a phenyl group substituted with a nitrile group.

The methyl group substituted with an alkoxy group may be represented by any one of the following chemical formulae.

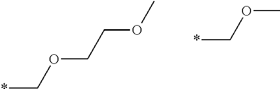

In the chemical formulae,

*- means a site linked to other substituents or linking sites.

The methyl group substituted with an alkoxy group may be further substituted with a methoxy group.

In one embodiment of the present specification, R′ and R″ are the same as or different from each other, and each independently hydrogen; a methyl group; a methyl group substituted with a phenyl group substituted with a nitro group; a methyl group substituted with a methoxy group; an ethyl group; a cyclohexyl group; a phenyl group substituted with a nitro group; or a phenyl group, or bond to each other to form a morpholinyl group.

In one embodiment of the present specification, Rx is hydrogen.

In one embodiment of the present specification, R3, R4, R10 and R11 are the same as or different from each other, and each independently hydrogen; a substituted or unsubstituted alkyl group having 1 to 30 carbon atoms; or a substituted or unsubstituted cycloalkyl group having 3 to 30 carbon atoms.

In one embodiment of the present specification, R3, R4, R10 and R11 are the same as or different from each other, and each independently hydrogen; a substituted or unsubstituted alkyl group having 1 to 20 carbon atoms; or a substituted or unsubstituted cycloalkyl group having 3 to 20 carbon atoms.

In one embodiment of the present specification, R3, R4, R10 and R11 are the same as or different from each other, and each independently hydrogen; a substituted or unsubstituted alkyl group having 1 to 10 carbon atoms; or a substituted or unsubstituted cycloalkyl group having 3 to 10 carbon atoms.

In one embodiment of the present specification, R3, R4, R10 and R11 are the same as or different from each other, and each independently hydrogen; a substituted or unsubstituted methyl group; a substituted or unsubstituted isopropyl group; a substituted or unsubstituted cyclopentyl group; or a substituted or unsubstituted cyclohexyl group.

In one embodiment of the present specification, R3, R4, R10 and R11 are the same as or different from each other, and each independently hydrogen; a methyl group; an isopropyl group; a cyclopentyl group substituted with a methyl group; or a cyclohexyl group unsubstituted or substituted with a methyl group or an isopropyl group.

In one embodiment of the present specification, R7 and R14 are the same as or different from each other, and each independently hydrogen; a substituted or unsubstituted phenyl group; a substituted or unsubstituted biphenyl group; a substituted or unsubstituted naphthyl group; a substituted or unsubstituted pyridinyl group; or a substituted or unsubstituted dibenzofuranyl group.

In one embodiment of the present specification, R7 and R14 are the same as or different from each other, and each independently hydrogen; a phenyl group unsubstituted or substituted with fluorine, a nitrile group, a methyl group, a trifluoromethyl group, a tert-butyl group or a methoxy group; a substituted or unsubstituted biphenyl group; a substituted or unsubstituted naphthyl group; a substituted or unsubstituted pyridinyl group; or a substituted or unsubstituted dibenzofuranyl group.

In one embodiment of the present specification, R7 and R14 are the same as or different from each other, and each independently hydrogen; a phenyl group unsubstituted or substituted with fluorine, a nitrile group, a methyl group, a trifluoromethyl group, a tert-butyl group or a methoxy group; a biphenyl group substituted with a methyl group; a naphthyl group; a pyridinyl group; or a dibenzofuranyl group.

In one embodiment of the present specification, at least one of R3, R4, R10 and R11 is a substituted or unsubstituted alkoxy group; a substituted or unsubstituted aryloxy group; a substituted or unsubstituted heteroaryloxy group; a substituted or unsubstituted arylthio group; a substituted or unsubstituted heteroarylthio group; or a substituted or unsubstituted alkylthio group.

In one embodiment of the present specification, at least one of R3, R4, R10 and R11 is a substituted or unsubstituted alkoxy group having 1 to 30 carbon atoms; a substituted or unsubstituted aryloxy group having 6 to 30 carbon atoms; a substituted or unsubstituted heteroaryloxy group having 2 to 30 carbon atoms; a substituted or unsubstituted arylthio group having 6 to 30 carbon atoms; a substituted or unsubstituted heteroarylthio group having 2 to 30 carbon atoms; or a substituted or unsubstituted alkylthio group having 1 to 30 carbon atoms.

In one embodiment of the present specification, at least one of R3, R4, R10 and R11 is a substituted or unsubstituted alkoxy group having 1 to 20 carbon atoms; a substituted or unsubstituted aryloxy group having 6 to 20 carbon atoms; a substituted or unsubstituted heteroaryloxy group having 2 to 20 carbon atoms; a substituted or unsubstituted arylthio group having 6 to 20 carbon atoms; a substituted or unsubstituted heteroarylthio group having 2 to 20 carbon atoms; or a substituted or unsubstituted alkylthio group having 1 to 20 carbon atoms.

In one embodiment of the present specification, at least one of R3, R4, R10 and R11 is a substituted or unsubstituted alkoxy group having 1 to 10 carbon atoms; a substituted or unsubstituted aryloxy group having 6 to 12 carbon atoms; a substituted or unsubstituted heteroaryloxy group having 2 to 10 carbon atoms; a substituted or unsubstituted arylthio group having 6 to 12 carbon atoms; a substituted or unsubstituted heteroarylthio group having 2 to 10 carbon atoms; or a substituted or unsubstituted alkylthio group having 1 to 10 carbon atoms.

In one embodiment of the present specification, at least one of R3, R4, R10 and R11 is a substituted or unsubstituted phenyloxy group; a substituted or unsubstituted isopropoxy group; a substituted or unsubstituted ethoxy group; a substituted or unsubstituted phenylthio group; a substituted or unsubstituted pyridinethio group; a substituted or unsubstituted isopropylthio group; or a substituted or unsubstituted pyridinoxy group.

In one embodiment of the present specification, at least one of R3, R4, R10 and R11 is a phenyloxy group unsubstituted or substituted with an ethyl group, a tert-butyl group, a methoxy group, fluorine, a nitrile group, a nitro group or a trifluoromethyl group; an isopropoxy group substituted with fluorine; an ethoxy group substituted with fluorine; a phenylthio group substituted with chlorine; a pyridinethio group; an isopropylthio group; or a pyridinoxy group.

In one embodiment of the present specification, R1, R6, R8 and R13 are the same as or different from each other, and each independently hydrogen; a methyl group; a cyclopentyl group; a cyclohexyl group; an isopropylthio group; a phenyloxy group substituted with a nitrile group; or a phenylthio group substituted with chlorine.

In one embodiment of the present specification, R2, R5, R9 and R12 are the same as or different from each other, and each independently hydrogen; a halogen group; a nitrile group; a substituted or unsubstituted phenyl group; —(C═O)NR′R″; or —(C═O)OR′″.

In one embodiment of the present specification, R2, R5, R9 and R12 are the same as or different from each other, and each independently hydrogen; chlorine; fluorine; bromine; iodine; a nitrile group; a substituted or unsubstituted phenyl group; —(C═O)NR′R″; or —(C═O)OR′″.

In one embodiment of the present specification, R2, R5, R9 and R12 are the same as or different from each other, and each independently hydrogen; chlorine; fluorine; bromine; iodine; a nitrile group; a phenyl group substituted with a trifluoromethyl group; —(C═O)NR′R″; or —(C═O)OR′″.

In one embodiment of the present specification, R′, R″ and R′″ are the same as or different from each other, and each independently a substituted or unsubstituted alkyl group; or a substituted or unsubstituted heteroaryl group.

In one embodiment of the present specification, R′, R″ and R′″ are the same as or different from each other, and each independently a substituted or unsubstituted alkyl group having 1 to 30 carbon atoms; or a substituted or unsubstituted heteroaryl group having 3 to 30 carbon atoms.

In one embodiment of the present specification, R′, R″ and R′″ are the same as or different from each other, and each independently a substituted or unsubstituted alkyl group having 1 to 20 carbon atoms; or a substituted or unsubstituted heteroaryl group having 3 to 20 carbon atoms.

In one embodiment of the present specification, R′, R″ and R′″ are the same as or different from each other, and each independently a substituted or unsubstituted alkyl group having 1 to 10 carbon atoms; or a substituted or unsubstituted heteroaryl group having 3 to 10 carbon atoms.

In one embodiment of the present specification, R′, R″ and R′″ are the same as or different from each other, and each independently a substituted or unsubstituted methyl group; a substituted or unsubstituted ethyl group; or a substituted or unsubstituted coumarin group.

In one embodiment of the present specification, R′, R″ and R′″ are the same as or different from each other, and each independently a methyl group; a methyl group substituted with a phenyl group substituted with a nitro group; an ethyl group; or a coumarin group.

In one embodiment of the present specification, R is a methyl group substituted with a phenyl group substituted with a nitro group; an ethyl group; or a coumarin group.

In one embodiment of the present specification, R′ and R″ are a methyl group.

In one embodiment of the present specification, R7 and R14 are the same as or different from each other, and each independently hydrogen; deuterium; a substituted or unsubstituted alkyl group; a substituted or unsubstituted aryl group; or a substituted or unsubstituted heteroaryl group.

In one embodiment of the present specification, R7 and R14 are the same as or different from each other, and each independently hydrogen; deuterium; a substituted or unsubstituted alkyl group having 1 to 30 carbon atoms; a substituted or unsubstituted aryl group having 6 to 30 carbon atoms; or a substituted or unsubstituted heteroaryl group having 3 to 30 carbon atoms.

In one embodiment of the present specification, R7 and R14 are the same as or different from each other, and each independently hydrogen; deuterium; a substituted or unsubstituted alkyl group having 1 to 20 carbon atoms; a substituted or unsubstituted aryl group having 6 to 20 carbon atoms; or a substituted or unsubstituted heteroaryl group having 3 to 20 carbon atoms.

In one embodiment of the present specification, R7 and R14 are the same as or different from each other, and each independently hydrogen; deuterium; a substituted or unsubstituted alkyl group having 1 to 10 carbon atoms; a substituted or unsubstituted aryl group having 6 to 12 carbon atoms; or a substituted or unsubstituted heteroaryl group having 3 to 10 carbon atoms.

In one embodiment of the present specification, R7 and R14 are the same as or different from each other, and each independently hydrogen; a substituted or unsubstituted methyl group; a substituted or unsubstituted phenyl group; a substituted or unsubstituted pyridinyl group; a substituted or unsubstituted dibenzofuranyl group; a substituted or unsubstituted quinolinyl group; a substituted or unsubstituted furanyl group; a substituted or unsubstituted thiophenyl group; or a substituted or unsubstituted benzofuranyl group.

In one embodiment of the present specification, R7 and R14 are the same as or different from each other, and each independently hydrogen; a methyl group; a trifluoromethyl group; a phenyl group unsubstituted or substituted with fluorine, a methyl group, a trifluoromethyl group, a tert-butyl group or a methoxy group; a pyridinyl group; a dibenzofuranyl group; a quinolinyl group; a furanyl group; a thiophenyl group; or a benzofuranyl group.

In one embodiment of the present specification, R7 and R14 are the same as or different from each other, and each independently hydrogen; a methyl group; a trifluoromethyl group; a phenyl group; a phenyl group substituted with fluorine; a phenyl group substituted with a methyl group; a phenyl group substituted with a trifluoromethyl group; a phenyl group substituted with a tert-butyl group; a phenyl group substituted with a methyl group and a methoxy group; a phenyl group substituted with a methyl group and a tert-butyl group; a pyridinyl group; a dibenzofuranyl group; a quinolinyl group; a furanyl group; a thiophenyl group; or a benzofuranyl group.

In one embodiment of the present specification, when R7 and R14 are hydrogen; a substituted or unsubstituted alkyl group; a substituted or unsubstituted aryl group; or a substituted or unsubstituted heteroaryl group and R2, R5, R9 and R12 are —C(═O)OR′″, R′″ is a substituted or unsubstituted aryl group; a substituted or unsubstituted cycloalkyl group; a substituted alkyl group; or a branched unsubstituted alkyl group having 3 to 30 carbon atoms.

In one embodiment of the present specification, when R2, R5, R9 and R12 are —C(═O)OR′″ and R′″ is a substituted alkyl group, it means an alkyl group substituted with at least one substituent other than an alkyl group.

In one embodiment of the present specification, when R2, R5, R9 and R12 are —C(═O)OR′″ and R′″ is a substituted alkyl group, it is an alkyl group substituted with a halogen group, a nitrile group, a nitro group, an alkyl group substituted with a halogen group, an alkyl group substituted with an alkoxy group, —C(═O)OR, —(C═O)NR′R″ or —CHO, and R, R′ and R″ are the same as described above.

In one embodiment of the present specification, when R2, R5, R9 and R12 are —C(═O)OR′″ and R′″ is a substituted alkyl group, it is an alkyl group having 1 to 30 carbon atoms substituted with an ethoxy group substituted with a methoxy group, a methyl group substituted with a methoxy group, a phenyl group substituted with a nitro group, a methoxy group, a trifluoromethyl group or a nitrile group.

In one embodiment of the present specification, when R2, R5, R9 and R12 are —C(═O)OR′″ and R′″ is a substituted alkyl group, it is an alkyl group having 1 to 20 carbon atoms substituted with an ethoxy group substituted with a methoxy group, a methyl group substituted with a methoxy group, a phenyl group substituted with a nitro group, a methoxy group, a trifluoromethyl group or a nitrile group.

In one embodiment of the present specification, when R2, R5, R9 and R12 are —C(═O)OR′″ and R′″ is a substituted alkyl group, it is an alkyl group having 1 to 10 carbon atoms substituted with an ethoxy group substituted with a methoxy group, a methyl group substituted with a methoxy group, a phenyl group substituted with a nitro group, a methoxy group, a trifluoromethyl group or a nitrile group.

In one embodiment of the present specification, when R2, R5, R9 and R12 are —C(═O)OR′″ and R′″ is a substituted alkyl group, it is an alkyl group having 1 to 5 carbon atoms substituted with an ethoxy group substituted with a methoxy group, a methyl group substituted with a methoxy group, a phenyl group substituted with a nitro group, a methoxy group, a trifluoromethyl group or a nitrile group.

In one embodiment of the present specification, when R2, R5, R9 and R12 are —C(═O)OR′″ and R′″ is a substituted alkyl group, it is a methyl group substituted with an ethoxy group substituted with a methoxy group, a phenyl group substituted with a nitro group, a methoxy group, a trifluoromethyl group or a nitrile group; or an n-propyl group substituted with a methyl group substituted with a methoxy group.

In one embodiment of the present specification, when R2, R5, R9 and R12 are —C(═O)OR′″ and R′″ is a branched unsubstituted alkyl group having 3 to 30 carbon atoms, R is preferably a 1-ethylpropyl group; or a 2-ethylhexyl group.

In one embodiment of the present specification, when R2, R5, R9 and R12 are —C(═O)OR′″, R′″ is a substituted or unsubstituted aryl group having 6 to 30 carbon atoms; a substituted or unsubstituted cycloalkyl group having 3 to 30 carbon atoms; a substituted alkyl group having 1 to 30 carbon atoms; or a branched unsubstituted alkyl group having 3 to 30 carbon atoms.

In one embodiment of the present specification, when R2, R5, R9 and R12 are —C(═O)OR′″, R′″ is a substituted or unsubstituted aryl group having 6 to 20 carbon atoms; a substituted or unsubstituted cycloalkyl group having 3 to 20 carbon atoms; a substituted alkyl group having 1 to 20 carbon atoms; or a branched unsubstituted alkyl group having 3 to 20 carbon atoms.

In one embodiment of the present specification, when R2, R5, R9 and R12 are —C(═O)OR′″, R′″ is a substituted or unsubstituted aryl group having 6 to 12 carbon atoms; a substituted or unsubstituted cycloalkyl group having 3 to 10 carbon atoms; a substituted alkyl group having 1 to 10 carbon atoms; or a branched unsubstituted alkyl group having 3 to 10 carbon atoms.

In one embodiment of the present specification, when R2, R5, R9 and R12 are —C(═O)OR′″, R′″ is a substituted or unsubstituted phenyl group; a substituted or unsubstituted cyclohexyl group; a substituted methyl group; a substituted ethyl group; a substituted n-propyl group; a 1-ethylpropyl group; or a 2-ethylhexyl group.

In one embodiment of the present specification, when R2, R5, R9 and R12 are —C(═O)OR′″, R′″ is a substituted or unsubstituted phenyl group; a substituted or unsubstituted cyclohexyl group; a substituted methyl group; a 1-ethylpropyl group; or a 2-ethylhexyl group.

In one embodiment of the present specification, when R2, R5, R9 and R12 are —C(═O)OR′″, R′″ is a phenyl group substituted with fluorine or a trifluoromethyl group; a cyclohexyl group substituted with one or more selected from the group consisting of a methyl group and an isopropyl group; a methyl group substituted with a nitrile group; an ethyl group substituted with fluorine; an isopropyl group substituted with fluorine; an n-propyl group substituted with a methyl group substituted with a methoxy group; a 1-ethylpropyl group; or a 2-ethylhexyl group.

In one embodiment of the present specification, Chemical Formula 1 may be represented by the following Chemical Formula 1-1.

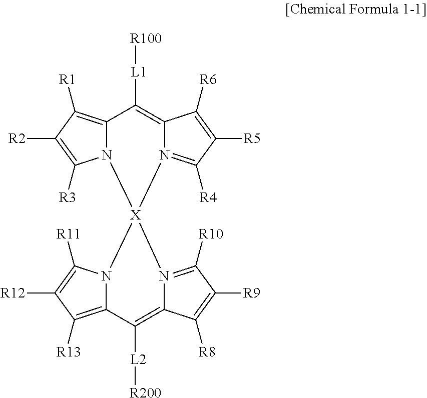

In Chemical Formula 1-1,

R1, R2, R5, R6, R8, R9, R12 and R13 are the same as or different from each other, and each independently hydrogen; a halogen group; an aldehyde group; a nitrile group; a nitro group; a substituted or unsubstituted ester group; a substituted or unsubstituted amide group; a substituted or unsubstituted alkyl group; a substituted or unsubstituted cycloalkyl group; a substituted or unsubstituted aryl group; a substituted or unsubstituted aryloxy group; a substituted or unsubstituted arylthio group; or a substituted or unsubstituted heteroaryl group,

R3, R4, R10 and R11 are each independently hydrogen; a substituted or unsubstituted alkyl group; or a substituted or unsubstituted cycloalkyl group,

L1 and L2 are each independently 0 or S,

R100 and R200 are each independently a substituted or unsubstituted aryl group; or a substituted or unsubstituted heteroaryl group, and

X is Zn, Co, Ni or Pd.

In one embodiment of the present specification, R1, R2, R5, R6, R8, R9, R12 and R13 are the same as or different from each other, and each independently hydrogen; a halogen group; a nitrile group; a nitro group; —CHO; —COOR; —(C═O)NR′R″; a linear or branched alkyl group; a fluoroalkyl group; a cycloalkyl group unsubstituted or substituted with an alkyl group; an aryl group unsubstituted or substituted with one or more substituents selected from among a nitrile group, a halogen group, an alkyl group and a fluoroalkyl group; a polycyclic heterocyclic group; an aryloxy group unsubstituted or substituted with one or more substituents selected from among a halogen group and an alkyl group; or an arylthio group unsubstituted or substituted with one or more substituents selected from among a halogen group and an alkyl group, and R, R′ and R″ are the same as or different from each other and each independently a substituted or unsubstituted alkyl group; a substituted or unsubstituted aryl group; or a substituted or unsubstituted heteroaryl group, and R′ and R″ may bond to each other to form a ring.

In one embodiment of the present specification, R1, R2, R5, R6, R8, R9, R12 and R13 are each hydrogen; a halogen group; a nitrile group; a nitro group; —CHO; —COOR; —(C═O)NR′R″; a C₁-C₁₀ alkyl group; a C₁-C₁₀ fluoroalkyl group; a C₃-C₁₂ cycloalkyl group; a C₅-C₁₅ alkylcycloalkyl group; an aryl group unsubstituted or substituted with one or more substituents selected from among a nitrile group, a halogen group, a C₁-C₁₀ alkyl group and a fluoroalkyl group; a C₆-C₁₀ aryloxy group unsubstituted or substituted with one or more substituents selected from among an alkyl group and a halogen group; a C₆-C₁₀ arylthio group unsubstituted or substituted with one or more substituents selected from among an alkyl group and a halogen group; or an O-including heteroaryl group, and R, R′ and R″ are each a C₁-C₁₀ alkyl group; a C₅-C₁₅ alkylaryl group; or a C₅-C₁₅ nitroaryl group.

In one embodiment of the present specification, R1, R2, R5, R6, R8, R9, R12 and R13 are each hydrogen; fluorine; chlorine; bromine; —CN; —NO₂; —CHO; —COOR; —(C═O)NR′R″; a C₁-C₅ alkyl group; a C₁-C₅ fluoroalkyl group; a C₃-C₈ cycloalkyl group; a C₅-C₁₀ alkylcycloalkyl group; a phenyl group unsubstituted or substituted with one or more substituents selected from among —CN, fluorine, a C₁-C₅ alkyl group and a trifluoroalkyl group; a phenoxy group unsubstituted or substituted with one or more substituents selected from among a C₁-C₅ alkyl group, fluorine, chlorine and bromine; a phenylthio group unsubstituted or substituted with one or more substituents selected from among a C₁-C₅ alkyl group, fluorine, chlorine and bromine; or an O-including polycyclic heteroaryl group, and R, R′ and R″ are each a C₁-C₅ alkyl group; a C₅-C₁₂ alkylaryl group; or a C₈-C₁₂ nitroaryl group.

In one embodiment of the present specification, R1, R2, R5, R6, R8, R9, R12 and R13 are each hydrogen; fluorine; chlorine; bromine; —CN; —NO₂; —CHO; —COOR; —(C═O)NR′R″; a methyl group; an ethyl group; a propyl group; a t-butyl group; —CF₃; a cyclopropyl group; a cyclobutyl group; a cyclopentyl group; a cyclohexyl group; a methylcyclohexyl group; a propylcyclohexyl group; a phenyl group unsubstituted or substituted with —CN, fluorine, a methyl group, an ethyl group, a propyl group, a t-butyl group or —CF₃; a phenoxy group substituted with a t-butyl group or fluorine; a phenylthio group substituted with chlorine; or a dibenzofuranyl group, and R, R′ and R″ are each a methyl group; an ethyl group; a t-butylphenyl group; or a nitrophenyl group.

In one embodiment of the present specification, Chemical Formula 1-1 is any one of the following Chemical Formulae 1-1-1 to 1-1-8.

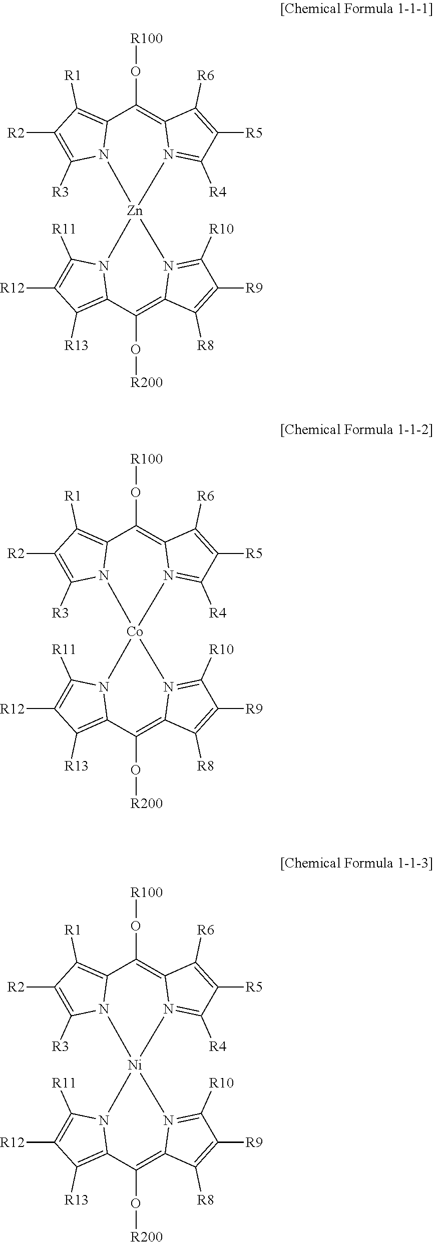

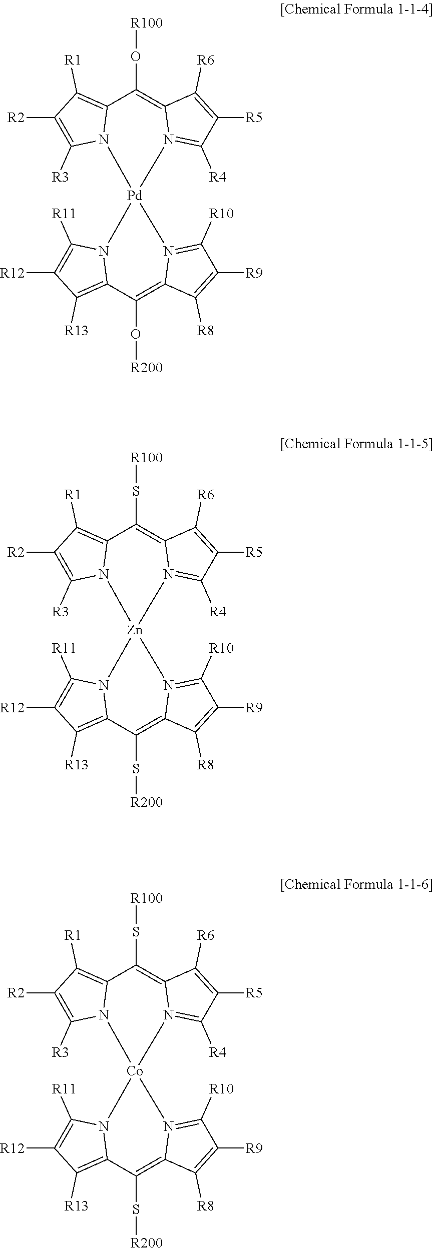

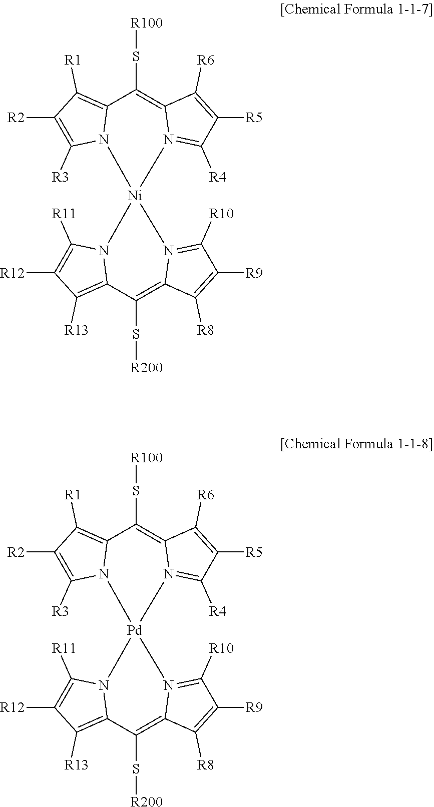

In Chemical Formulae 1-1-1 to 1-1-8,

each substituent has the same definition as in Chemical Formula 1-1.

In one embodiment of the present specification, R3, R4, R10 and R11 are the same as or different from each other, and each independently hydrogen; a linear or branched alkyl group; or a cycloalkyl group unsubstituted or substituted with an alkyl group.

In one embodiment of the present specification, R3, R4, R10 and R11 are each hydrogen.

In one embodiment of the present specification, R3, R4, R10 and R11 are each a C₁-C₁₀ alkyl group.

In one embodiment of the present specification, R3, R4, R10 and R11 are each a C₁-C₁₀ linear alkyl group.

In one embodiment of the present specification, R3, R4, R10 and R11 are each a C₁-C₅ alkyl group.

In one embodiment of the present specification, R3, R4, R10 and R11 are each a C₁-C₅ linear alkyl group.

In one embodiment of the present specification, R3, R4, R10 and R11 are each a C₁-C₃ alkyl group.

In one embodiment of the present specification, R3, R4, R10 and R11 are each a C₁-C₃ linear alkyl group.

In one embodiment of the present specification, R3, R4, R10 and R11 are each a methyl group or a propyl group.

In one embodiment of the present specification, R3, R4, R10 and R11 are each a cycloalkyl group unsubstituted or substituted with a C₁-C₁₀ alkyl group.

In one embodiment of the present specification, R3, R4, R10 and R11 are each a C₃-C₁₂ cycloalkyl group.

In one embodiment of the present specification, R3, R4, R10 and R11 are each a C₃-C₈ cycloalkyl group.

In one embodiment of the present specification, R3, R4, R10 and R11 are each a cyclopropyl group, a cyclobutyl group, a cyclopentyl group or a cyclohexyl group.

In one embodiment of the present specification, R3, R4, R10 and R11 are each a C₅-C₁₅ alkylcycloalkyl group.

In one embodiment of the present specification, R3, R4, R10 and R11 are each a C₅-C₁₀ alkylcycloalkyl group.

In one embodiment of the present specification, R3, R4, R10 and R11 are each a methylcyclohexyl group or a propylcyclohexyl group.

In one embodiment of the present specification, R100 and R200 are the same as or different from each other, and each independently an aryl group unsubstituted or substituted with one or more substituents selected from among an alkyl group, a fluoroalkyl group, a halogen group, a nitrile group, an alkoxy group and an aryl group; or a heteroaryl group unsubstituted or substituted with an alkyl group or ═O.

In one embodiment of the present specification, R100 and R200 are each an aryl group unsubstituted or substituted with one or more substituents selected from among a methyl group, an ethyl group, a propyl group, a t-butyl group, fluorine, a trifluoromethyl group, a nitrile group, a methoxy group and a phenyl group; or a heteroaryl group unsubstituted or substituted with a methyl group or ═O.

In one embodiment of the present specification, R100 and R200 are each a phenyl group unsubstituted or substituted with one or more substituents selected from among a methyl group, an ethyl group, a propyl group, a t-butyl group, fluorine, a trifluoromethyl group, a nitrile group, a methoxy group and a phenyl group; a dibenzofuranyl group; a pyridine group unsubstituted or substituted with a methyl group; or a benzopyranyl group unsubstituted or substituted with ═O.

In one embodiment of the present specification, examples of the benzopyranyl group substituted with ═O may include a coumarin group, a chromone group and the like.

In one embodiment of the present specification, Chemical Formula 1 is represented by the following Chemical Formula 2-1.

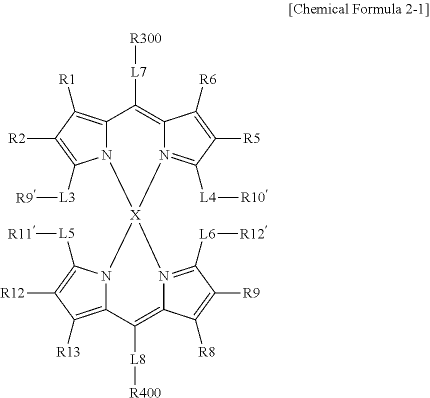

In Chemical Formula 2-1,

R1, R2, R5, R6, R8, R9, R12 and R13 are the same as or different from each other, and each independently hydrogen; a halogen group; an aldehyde group; a nitrile group; a nitro group; a substituted or unsubstituted ester group; a substituted or unsubstituted amide group; a substituted or unsubstituted alkyl group; a substituted or unsubstituted cycloalkyl group; a substituted or unsubstituted aryl group; a substituted or unsubstituted aryloxy group; a substituted or unsubstituted arylthio group; or a substituted or unsubstituted heteroaryl group,

R9′ to R12′ are the same as or different from each other, and each independently a substituted or unsubstituted alkyl group; a substituted or unsubstituted aryl group; or a substituted or unsubstituted heteroaryl group,

L3 to L6, L7 and L8 are each independently O or S,

R300 and R400 are each independently a substituted or unsubstituted aryl group; or a substituted or unsubstituted heteroaryl group, and

X is Zn, Co, Ni or Pd.

In one embodiment of the present specification, R1, R2, R5, R6, R8, R9, R12 and R13 are the same as or different from each other, and each independently hydrogen; a halogen group; a nitrile group; a nitro group; —CHO; —COOR; —(C═O)NR′R″; a linear or branched alkyl group; a fluoroalkyl group; a cycloalkyl group unsubstituted or substituted with an alkyl group; an aryl group unsubstituted or substituted with one or more substituents selected from among a nitrile group, a halogen group, an alkyl group and a fluoroalkyl group; a polycyclic heterocyclic group; an aryloxy group unsubstituted or substituted with one or more substituents selected from among a halogen group, an alkyl group and a fluoroalkyl group; or an arylthio group unsubstituted or substituted with one or more substituents selected from among a halogen group and an alkyl group, and R, R′ and R″ are the same as or different from each other and each independently a substituted or unsubstituted alkyl group; a substituted or unsubstituted aryl group; or a substituted or unsubstituted heteroaryl group, and R′ and R″ may bond to each other to form a ring.

In one embodiment of the present specification, R1, R2, R5, R6, R8, R9, R12 and R13 are each hydrogen; a halogen group; a nitrile group; a nitro group; —CHO; —COOR; —(C═O)NR′R″; a C₁-C₁₀ alkyl group; a C₁-C₁₀ fluoroalkyl group; a C₃-C₁₂ cycloalkyl group; a C₅-C₁₅ alkylcycloalkyl group; an aryl group unsubstituted or substituted with a nitrile group, a halogen group, a C₁-C₁₀ alkyl group or a fluoroalkyl group; a C₆-C₁₀ aryloxy group unsubstituted or substituted with one or more substituents selected from among an alkyl group, a fluoroalkyl group and a halogen group; a C₆-C₁₀ arylthio group unsubstituted or substituted with one or more substituents selected from among an alkyl group and a halogen group; or an O-including heteroaryl group, and R, R′ and R″ are each a C₁-C₁₀ alkyl group; a C₂-C₁₀ alkoxyalkyl group; a C₅-C₁₅ alkylaryl group; a C₅-C₁₅ arylalkyl group unsubstituted or substituted with NO₂; or an O-including heteroaryl group unsubstituted or substituted with ═O, and R′ and R″ may bond to each other to form an O-including heteroring.

In one embodiment of the present specification, R1, R2, R5, R6, R8, R9, R12 and R13 are each hydrogen; fluorine; chlorine; bromine; —CN; —NO₂; —CHO; —COOR; —(C═O)NR′R″; a C₁-C₅ alkyl group; a C₁-C₅ fluoroalkyl group; a C₃-C₈ cycloalkyl group; a C₅-C₁₀ alkylcycloalkyl group; a phenyl group unsubstituted or substituted with one or more substituents selected from among —CN, fluorine, a C₁-C₅ alkyl group and a trifluoroalkyl group; a phenoxy group unsubstituted or substituted with one or more substituents selected from among a C₁-C₅ alkyl group, a trifluoroalkyl group, fluorine, chlorine and bromine; a phenylthio group unsubstituted or substituted with one or more substituents selected from among a C₁-C₅ alkyl group, fluorine, chlorine and bromine; or an O-including polycyclic heteroaryl group, and R, R′ and R″ are each a C₁-C₅ alkyl group; a C₂-C₅ alkoxyalkyl group; a C₅-C₁₂ alkylaryl group; a benzyl group unsubstituted or substituted with NO₂; or a benzopyranyl group unsubstituted or substituted with ═0, and when R′ and R″ are each a C₁-C₅ alkyl group and a C₂-C₅ alkoxyalkyl group, R′ and R″ may bond to each other to form an O-including heteroring.

In one embodiment of the present specification, R1, R2, R5, R6, R8, R9, R12 and R13 are each hydrogen; fluorine; chlorine; bromine; —CN; —NO₂; —CHO; —COOR; —(C═O)NR′R″; a methyl group; an ethyl group; a propyl group; a t-butyl group; —CF₃; a cyclopropyl group; a cyclopentyl group; a cyclohexyl group; a methylcyclohexyl group; a propylcyclohexyl group; a phenyl group unsubstituted or substituted with —CN, fluorine, a methyl group, an ethyl group, a propyl group, a t-butyl group or —CF₃; a phenoxy group substituted with a t-butyl group, —CF₃ or fluorine; a phenylthio group substituted with chlorine; or a dibenzofuranyl group, and R, R′ and R″ are each a methyl group; an ethyl group; a methoxyethyl group; a t-butylphenyl group; a nitrobenzyl group; or a coumarin group, and by R′ and R″ bonding to each other to form an O-including heteroring, NR′R″ may become a morpholinyl group.

In one embodiment of the present specification, Chemical Formula 2-1 is any one of the following Chemical Formulae 2-1-1 to 2-1-8.

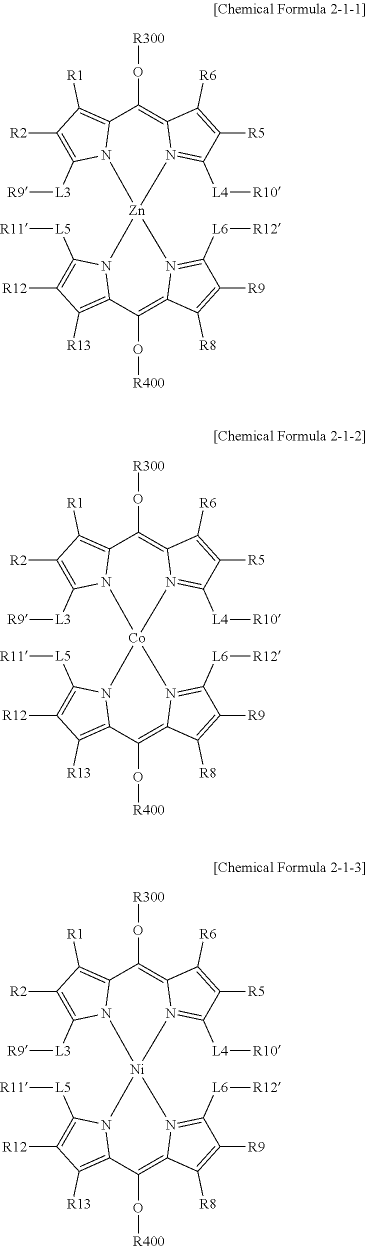

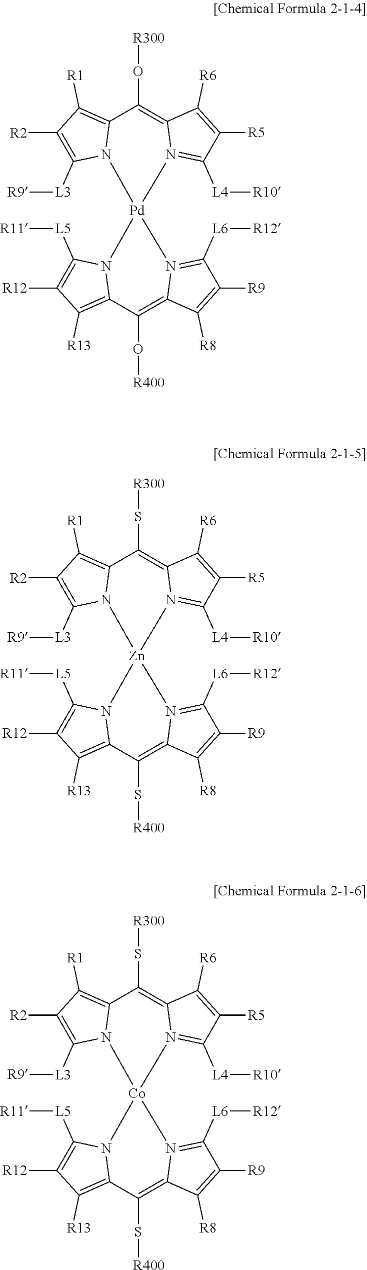

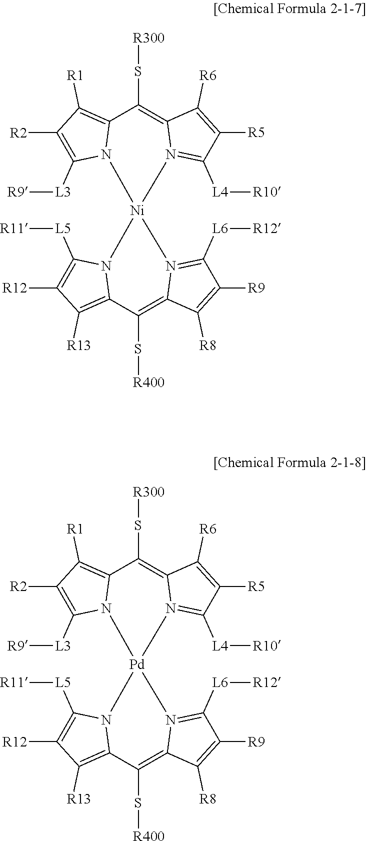

In Chemical Formulae 2-1-1 to 2-1-8, each substituent has the same definition as in Chemical Formula 2-1.

In one embodiment of the present specification, R9′ to R12′ are the same as or different from each other, and each independently an alkyl group unsubstituted or substituted with a halogen group; an aryl group unsubstituted or substituted with one or more substituents selected from among a nitrile group, a nitro group, a halogen group, an alkyl group and an alkoxy group; or a substituted or unsubstituted heteroaryl group including one or more of N and O.

In one embodiment of the present specification, R9′ to R12′ are each an alkyl group unsubstituted or substituted with fluorine.

In one embodiment of the present specification, R9′ to R12′ are each a hexafluoropropanyl group.

In one embodiment of the present specification, R9′ to R12′ are each a perfluoroalkyl group.

In one embodiment of the present specification, R9′ to R12′ are each a perfluoroethyl group.

In one embodiment of the present specification, R9′ to R12′ are each a phenyl group unsubstituted or substituted with one or more substituents selected from among a nitrile group, a nitro group, a halogen group, a C₁-C₁₀ alkyl group, a C₁-C₁₀ fluoroalkyl group and a C₁-C₁₀ alkoxy group.

In one embodiment of the present specification, R9′ to R12′ are each a phenyl group unsubstituted or substituted with one or more substituents selected from among —CN, —NO₂, fluorine, chlorine, a methyl group, a t-butyl group, —CF₃ and a methoxy group.

In one embodiment of the present specification, R9′ to R12′ are each an N- or O-including heteroaryl group unsubstituted or substituted with ═0.

In one embodiment of the present specification, R9′ to R12′ are each an N-including monocyclic or polycyclic heteroaryl group; an O-including monocyclic or polycyclic heteroaryl group unsubstituted or substituted with ═0.

In one embodiment of the present specification, R9′ to R12′ are each an N-including monocyclic heteroaryl group; an O-including polycyclic heteroaryl group; or a benzopyranyl group unsubstituted or substituted with ═0.

In one embodiment of the present specification, R9′ to R12′ are each a pyridinyl group, a dibenzofuranyl group, a coumarin group or a chromone group.

In one embodiment of the present specification, R300 and R400 are the same as or different from each other, and each independently an aryl group unsubstituted or substituted with one or more substituents selected from among a nitrile group, a halogen group, an alkyl group, an alkoxy group, an aryl group and an alkoxyaryl group; or a substituted or unsubstituted heteroaryl group including one or more of N and O.

In one embodiment of the present specification, R300 and R400 are each a phenyl group unsubstituted or substituted with one or more substituents selected from among a nitrile group, a halogen group, a C₁-C₁₀ alkyl group, a C₁-C₁₀ alkoxy group, a C₆-C₁₀ aryl group and a C₇-C₁₅ alkoxyaryl group.

In one embodiment of the present specification, R300 and R400 are each a phenyl group unsubstituted or substituted with one or more substituents selected from among —CN, chlorine, fluorine, a methyl group, an ethyl group, a propyl group, a t-butyl group, a methoxy group, a phenyl group and a methoxyphenyl group.

In one embodiment of the present specification, R300 and R400 are each a naphthyl group.

In one embodiment of the present specification, R300 and R400 are each an N- or O-including heteroaryl group unsubstituted or substituted with ═0.

In one embodiment of the present specification, R300 and R400 are each an N-including polycyclic heteroaryl group; or a benzopyranyl group unsubstituted or substituted with ═O.

In one embodiment of the present specification, R300 and R400 are each a carbazole group, a coumarin group or a chromone group.

In one embodiment of the present specification, the compound represented by Chemical Formula 1 may be represented by any one of the following compounds.

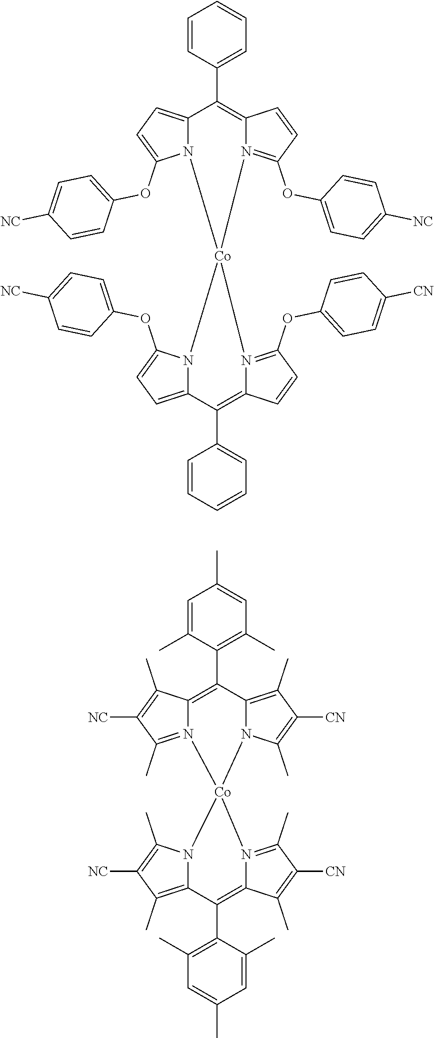

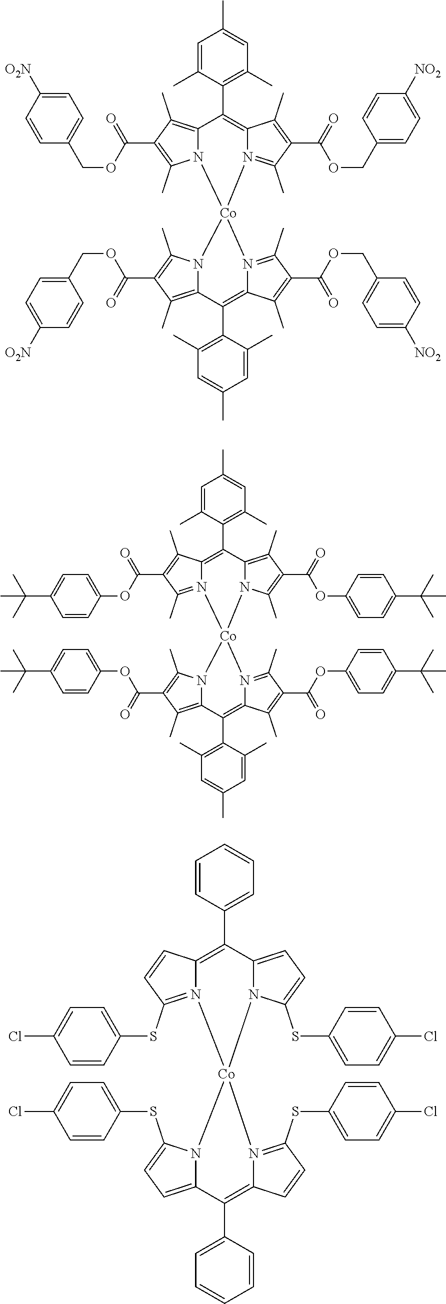

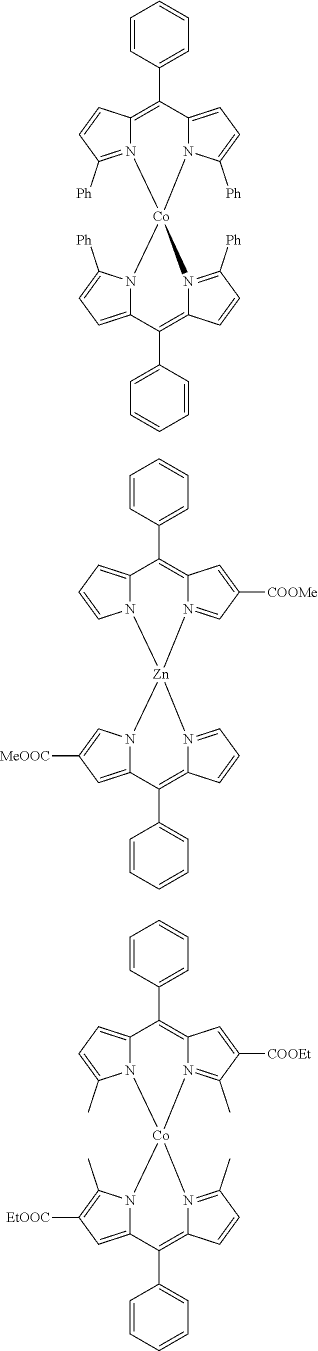

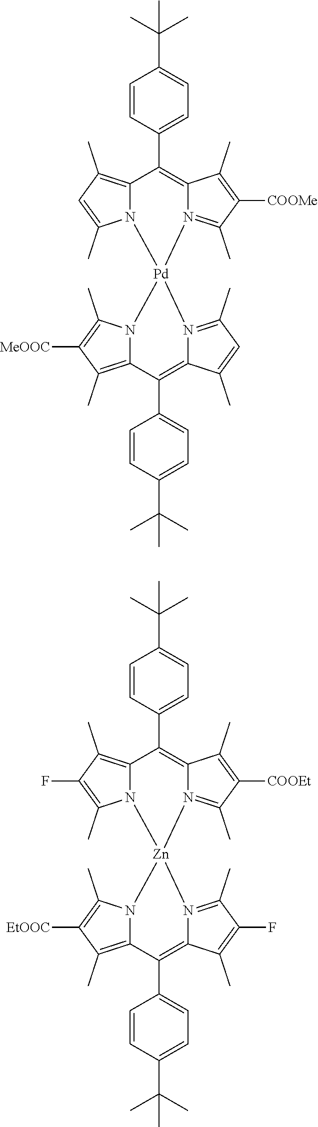

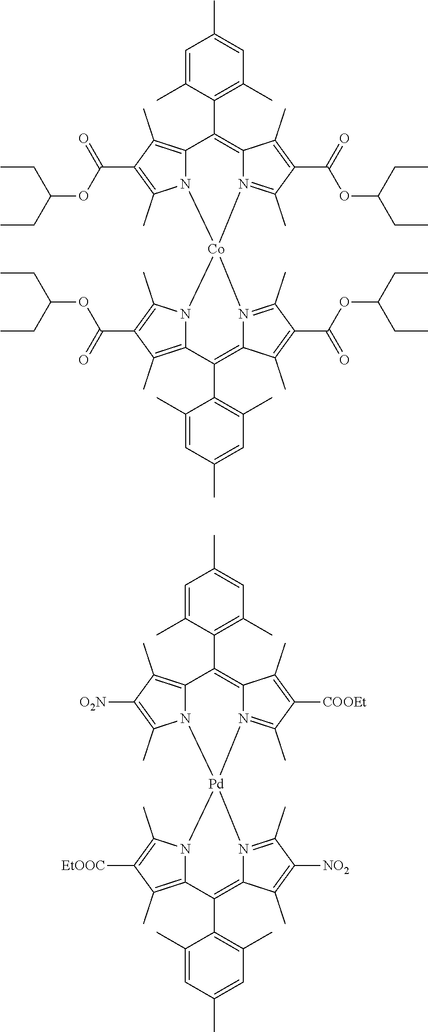

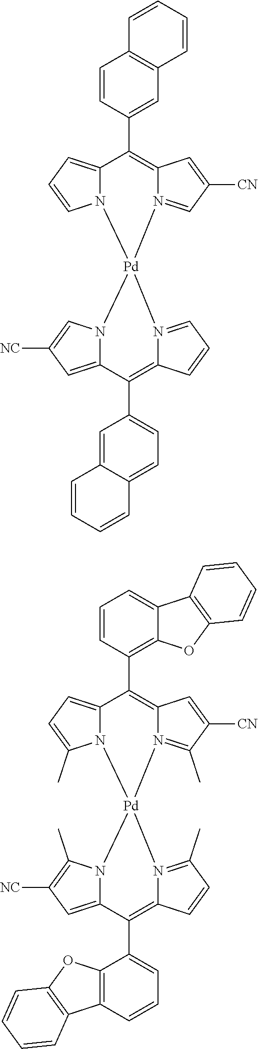

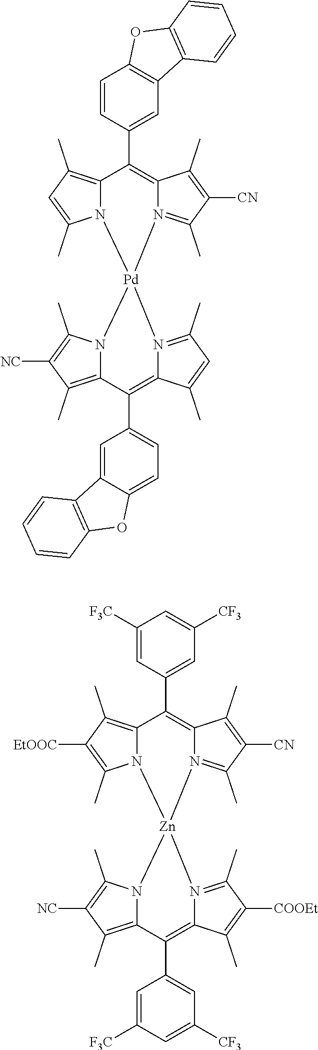

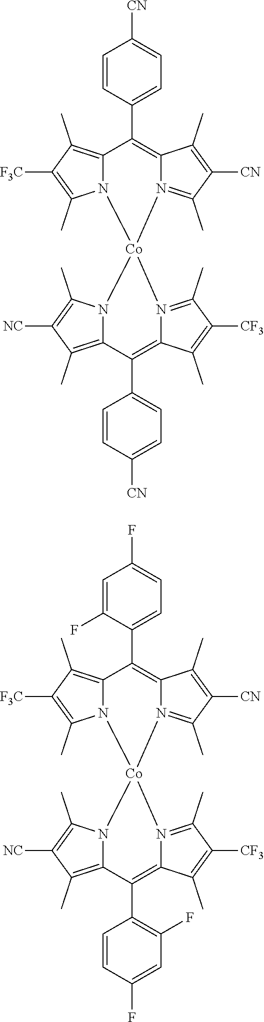

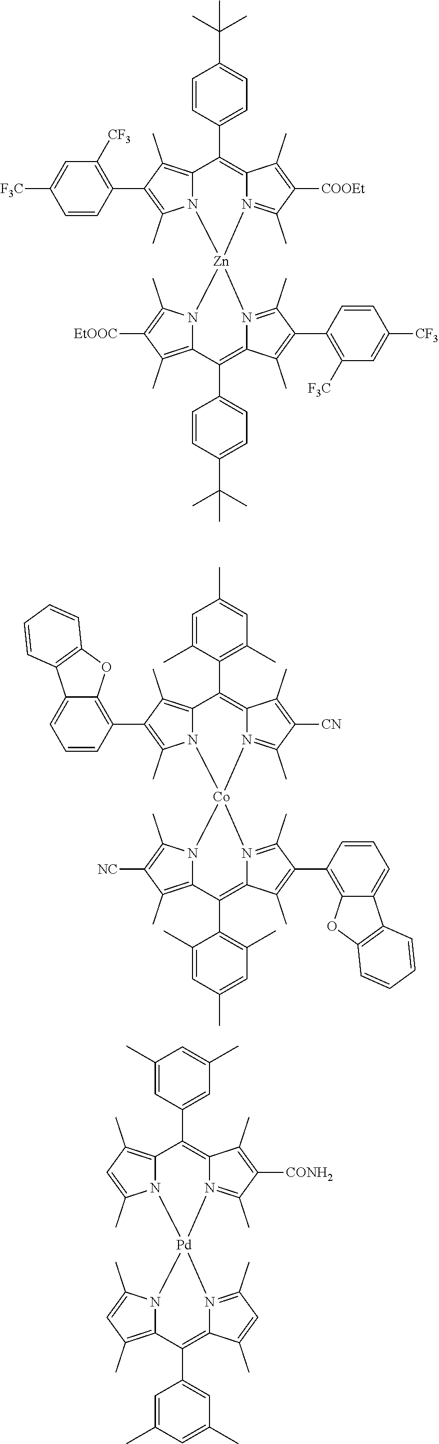

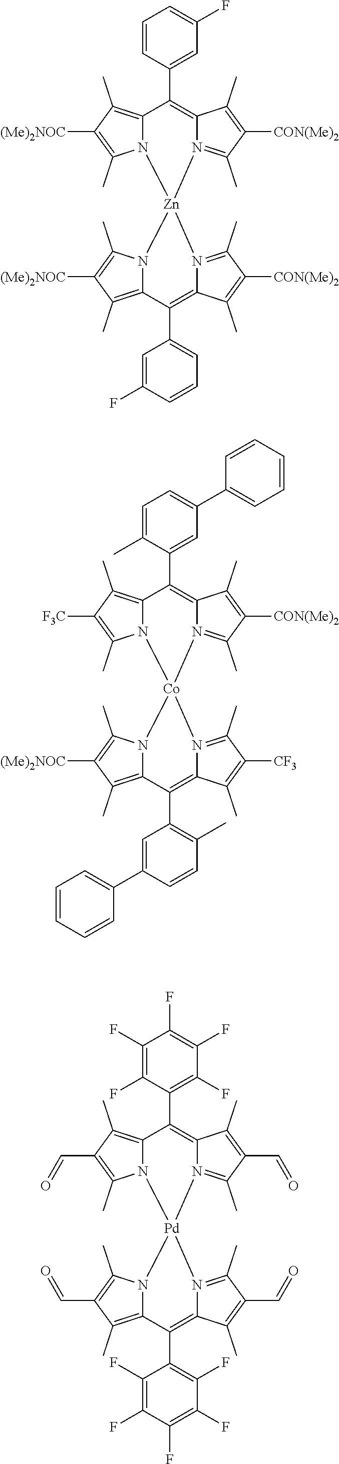

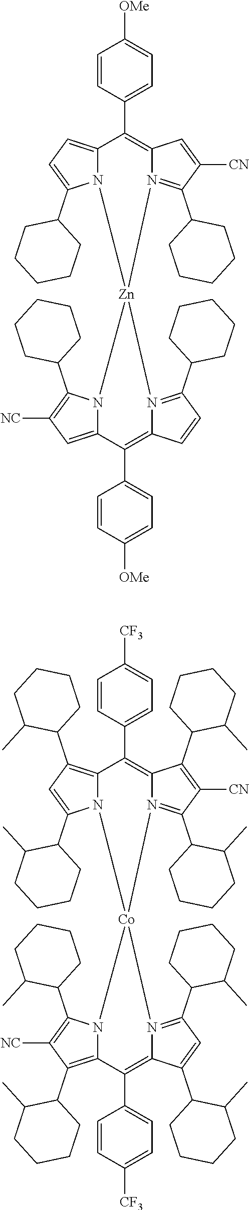

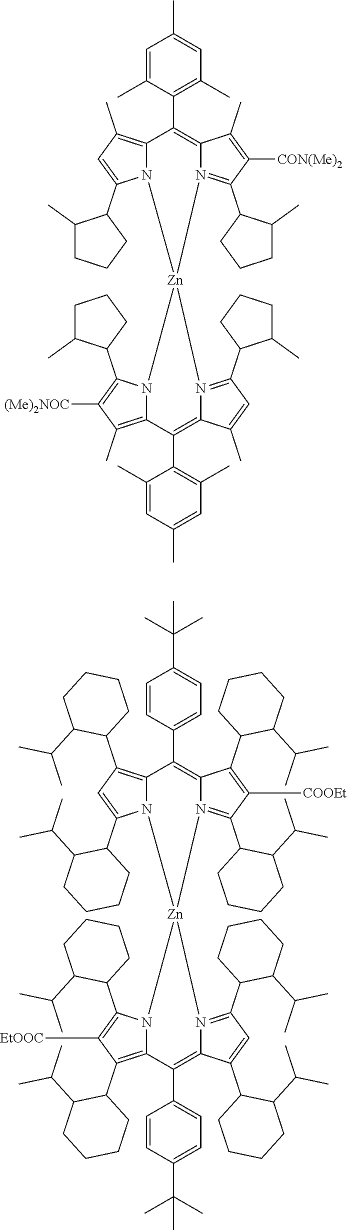

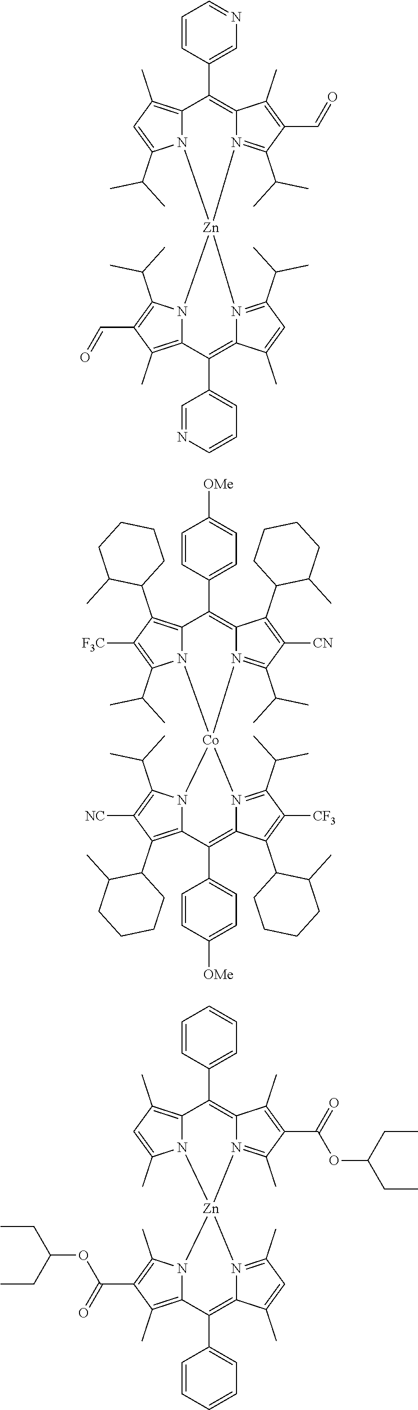

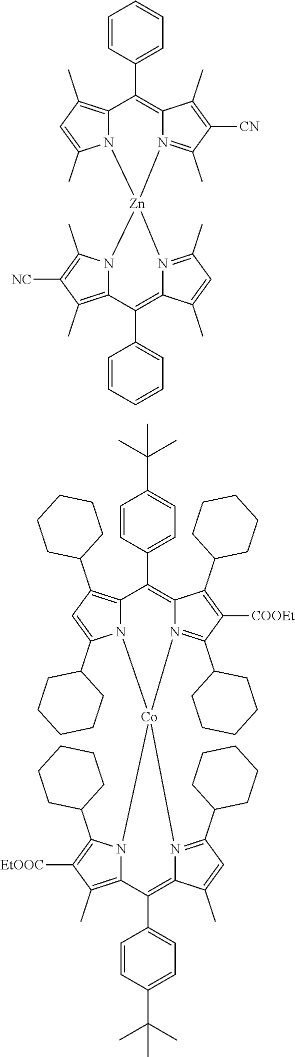

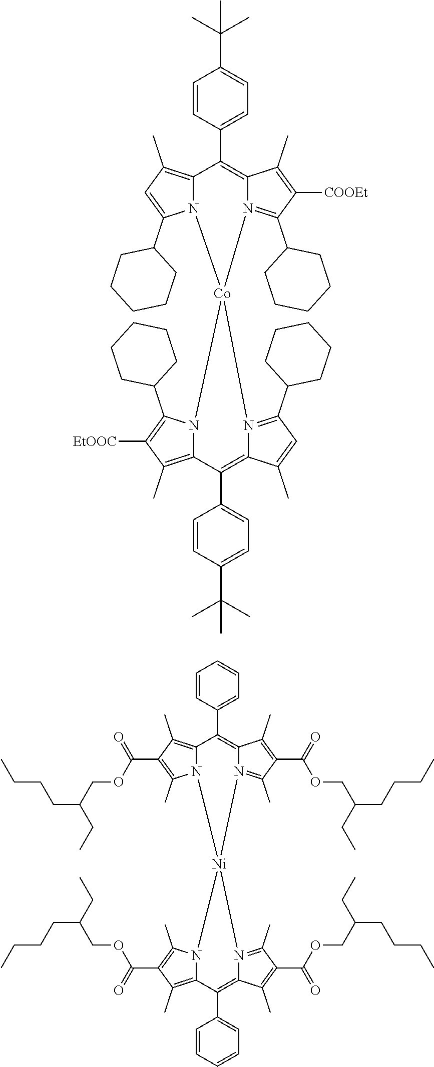

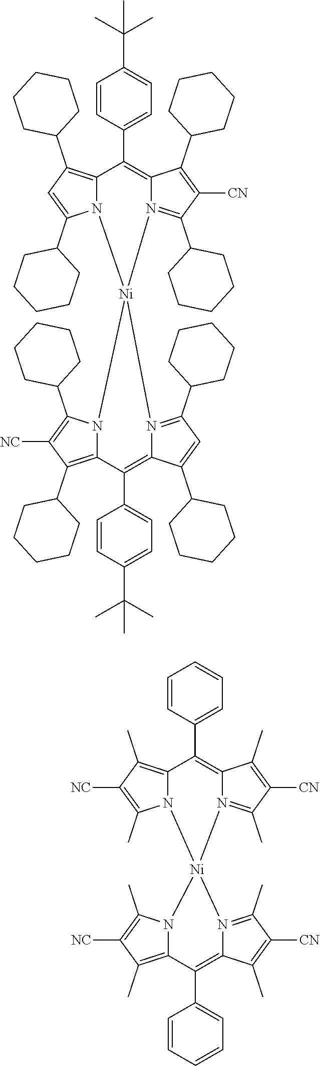

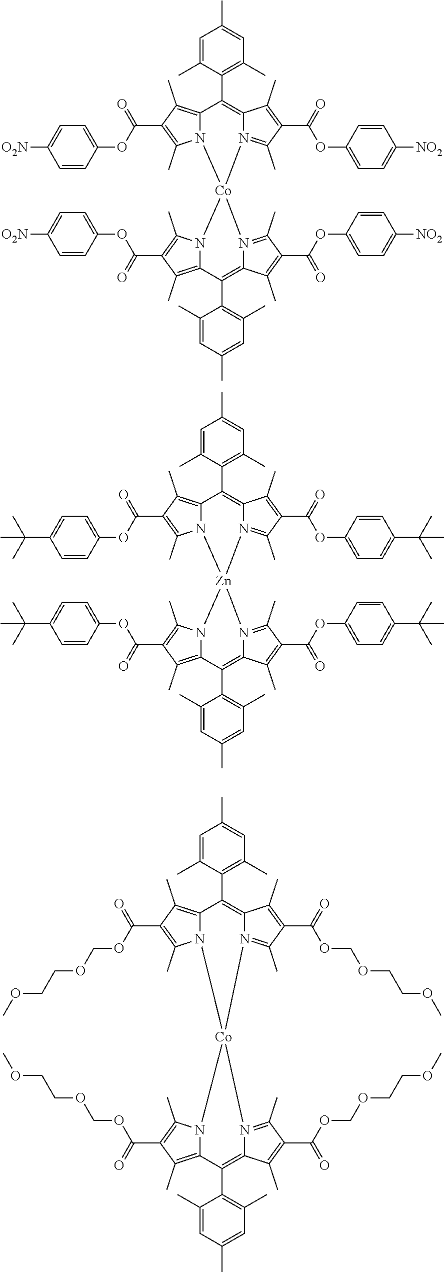

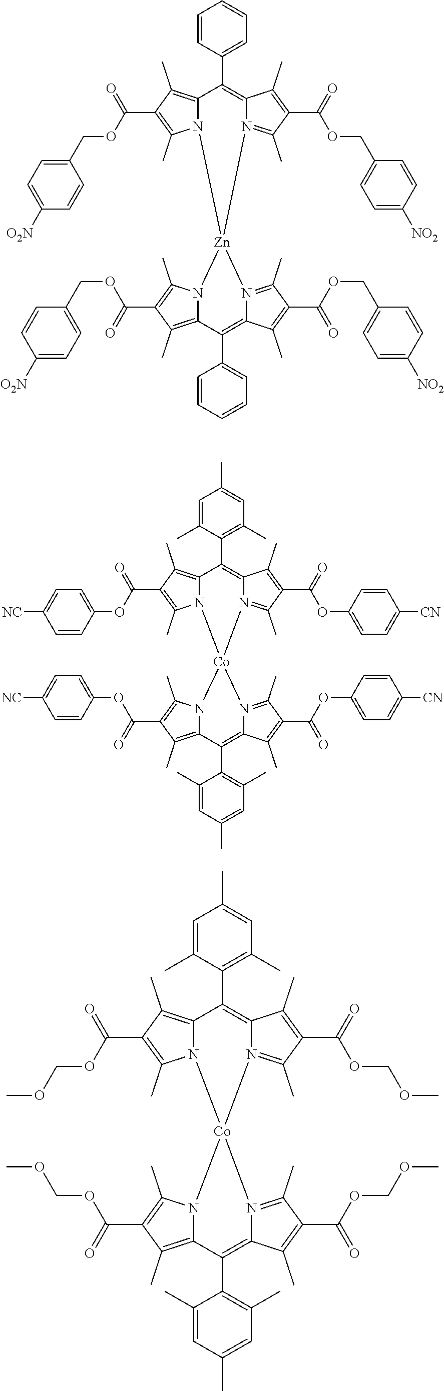

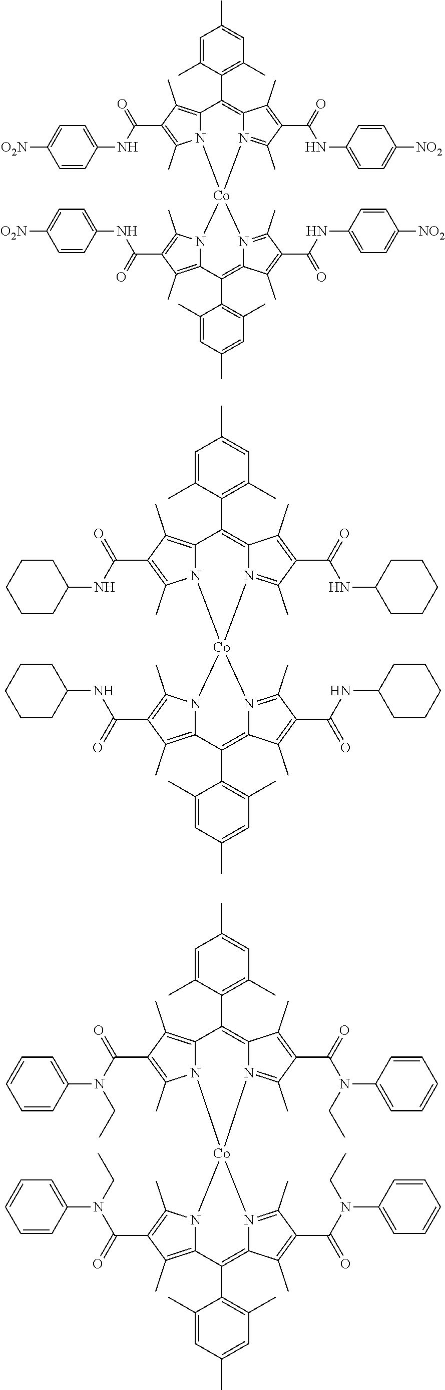

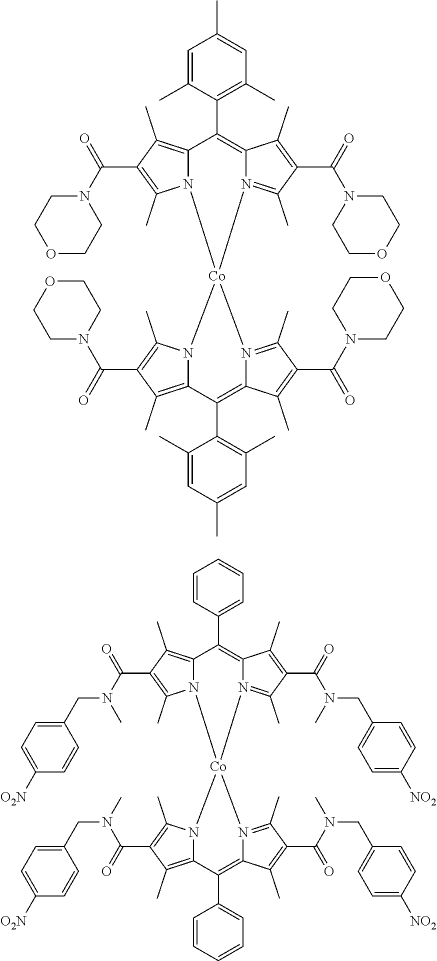

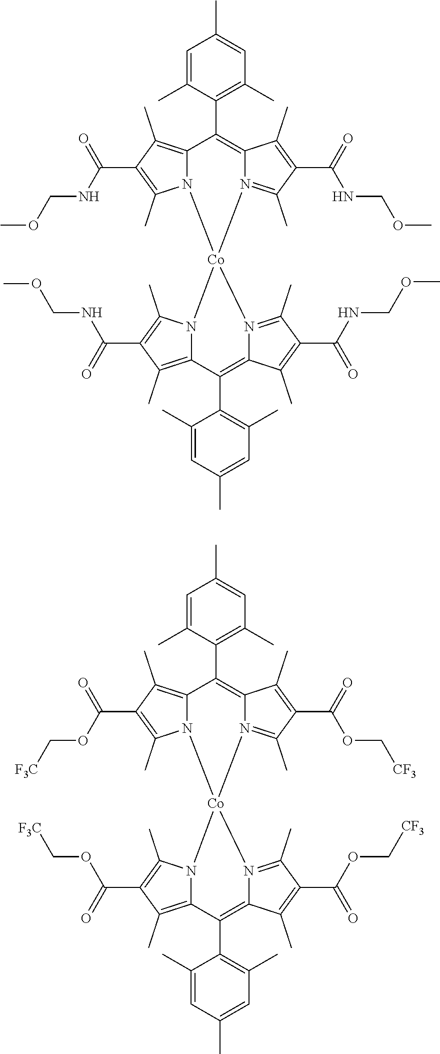

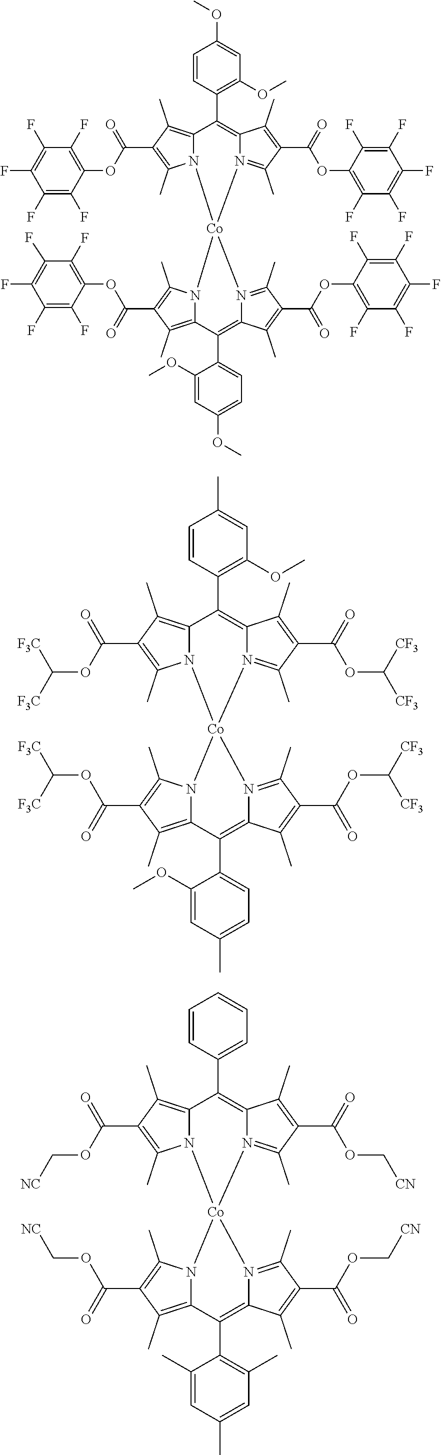

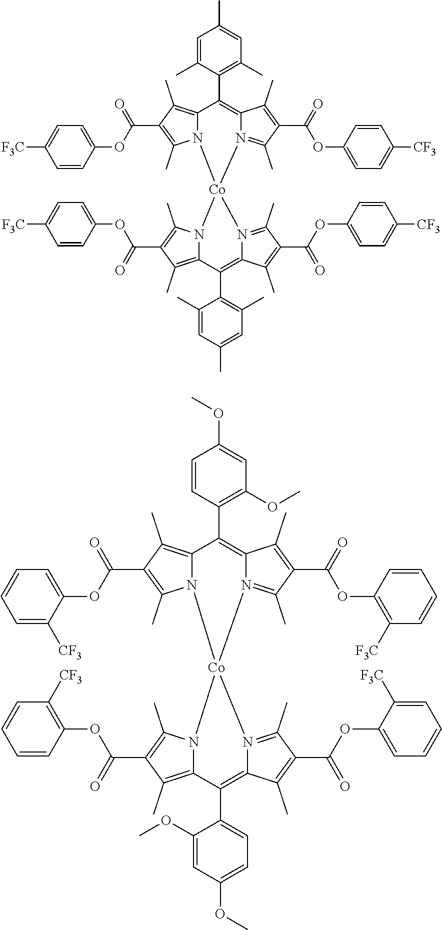

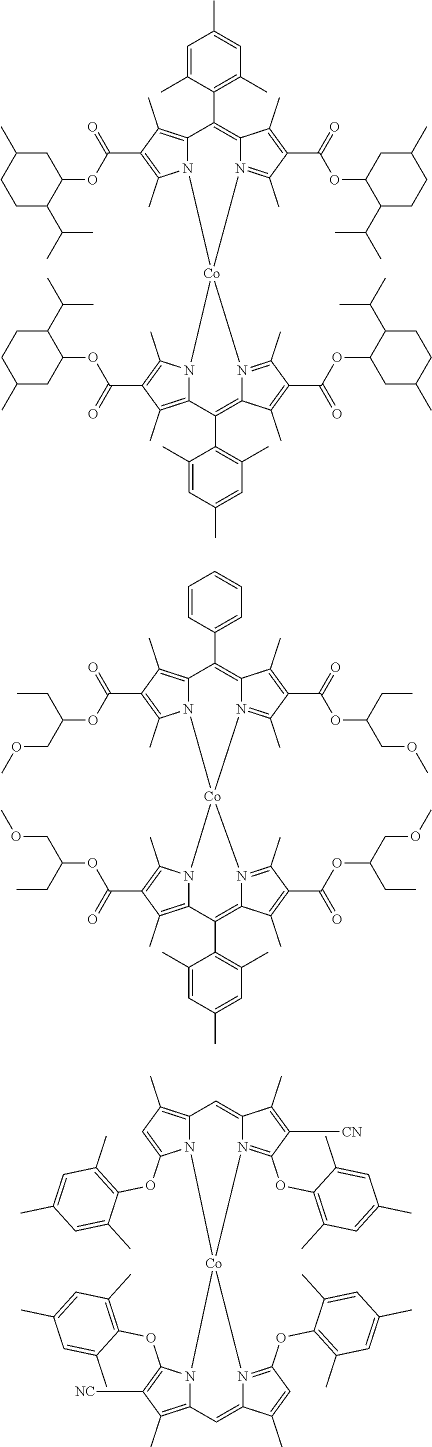

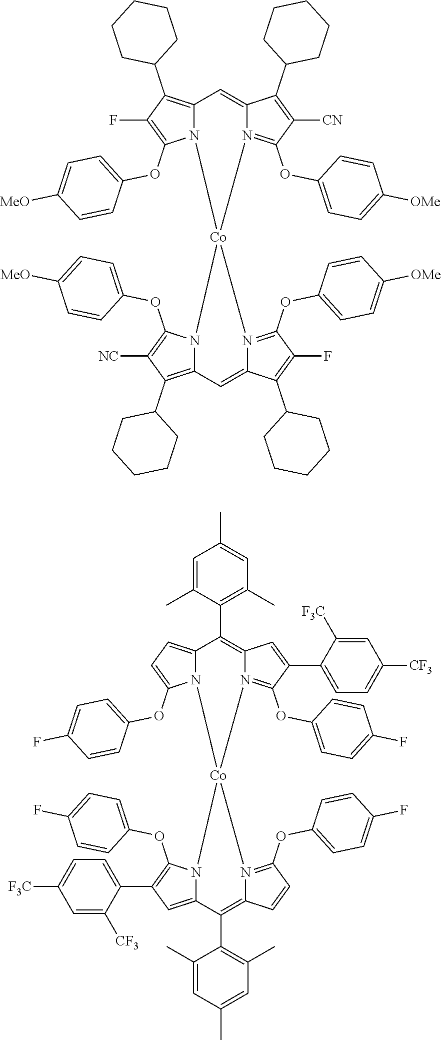

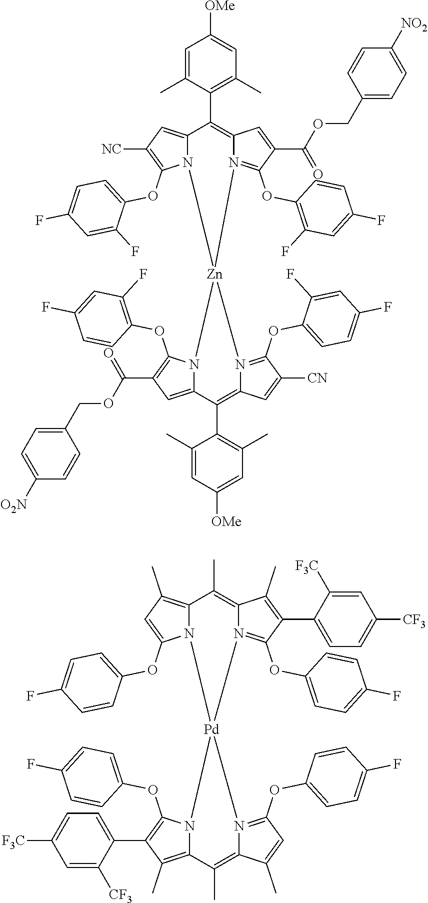

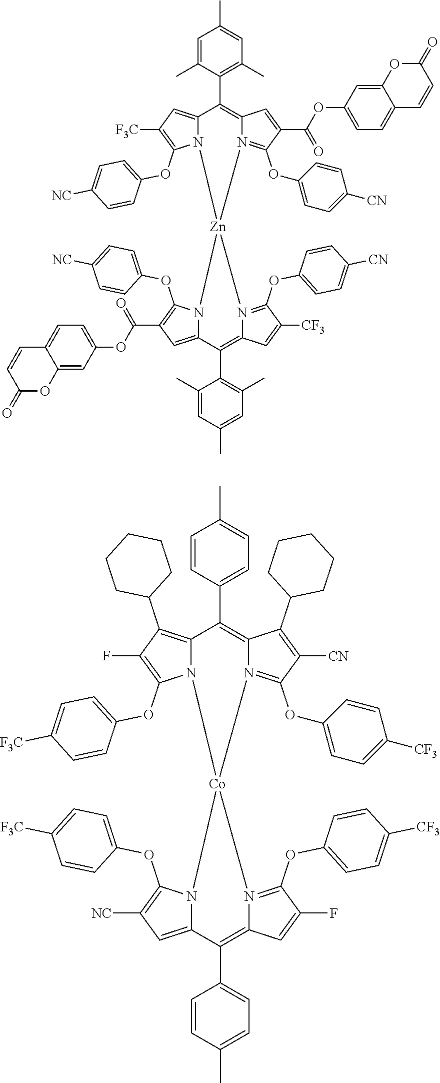

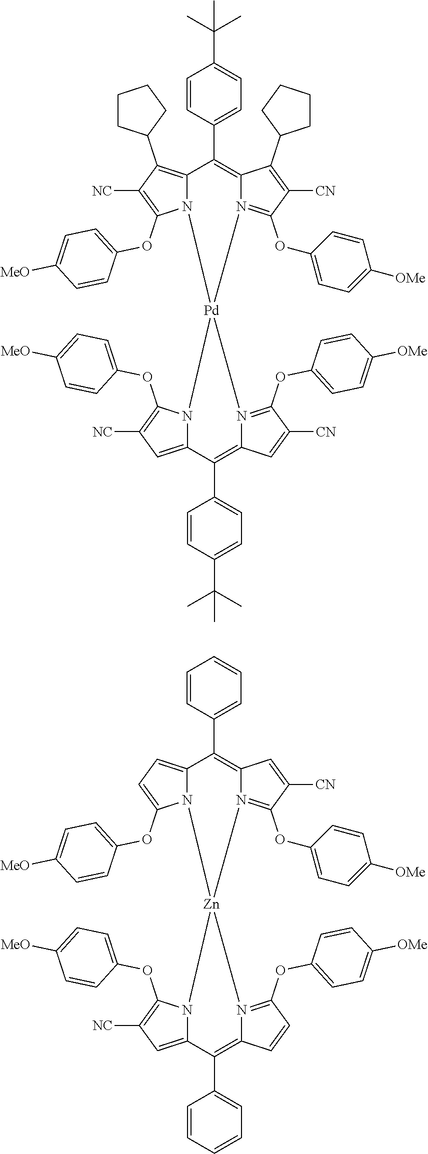

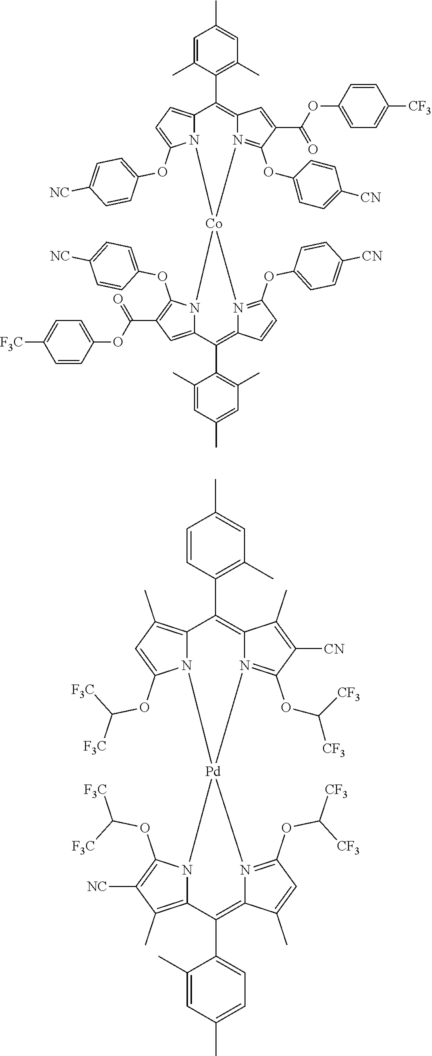

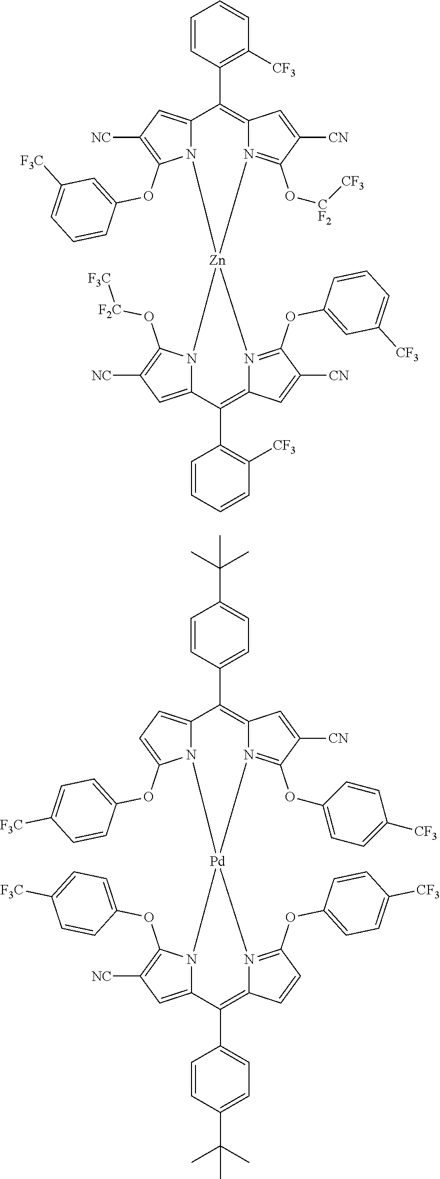

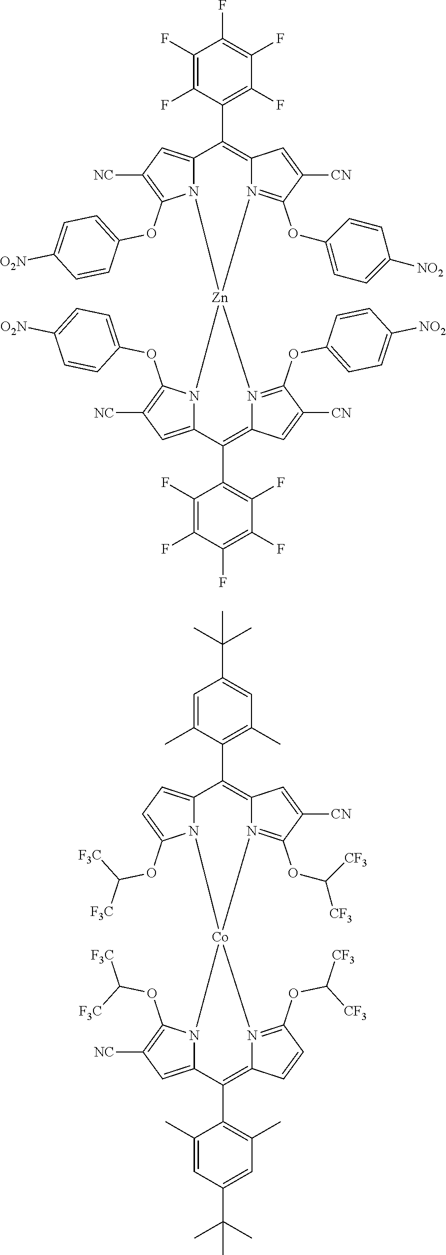

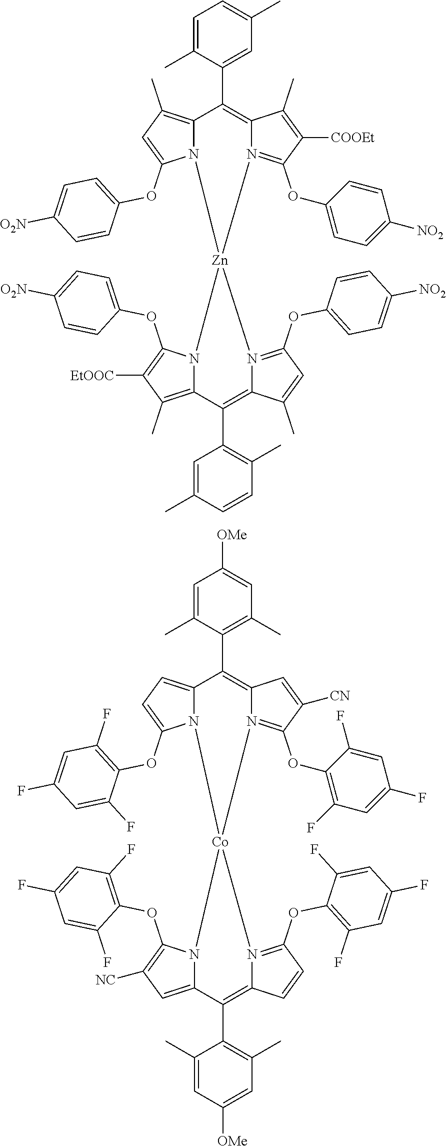

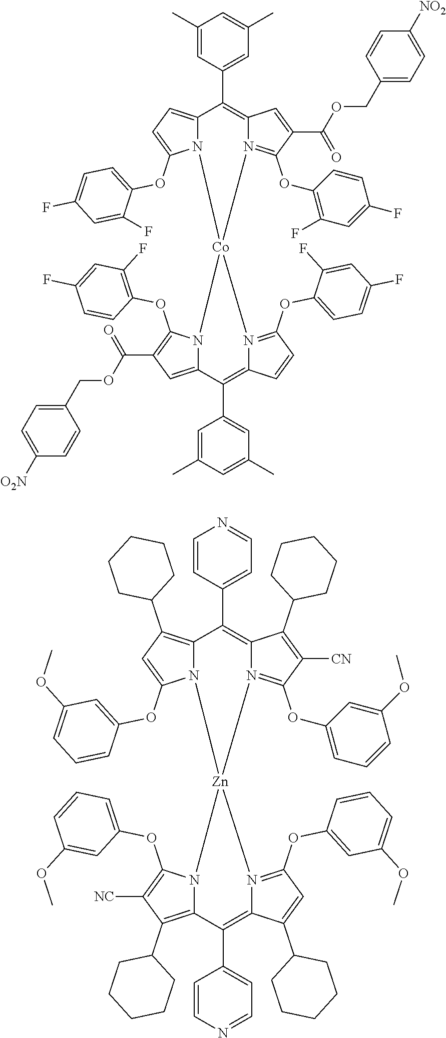

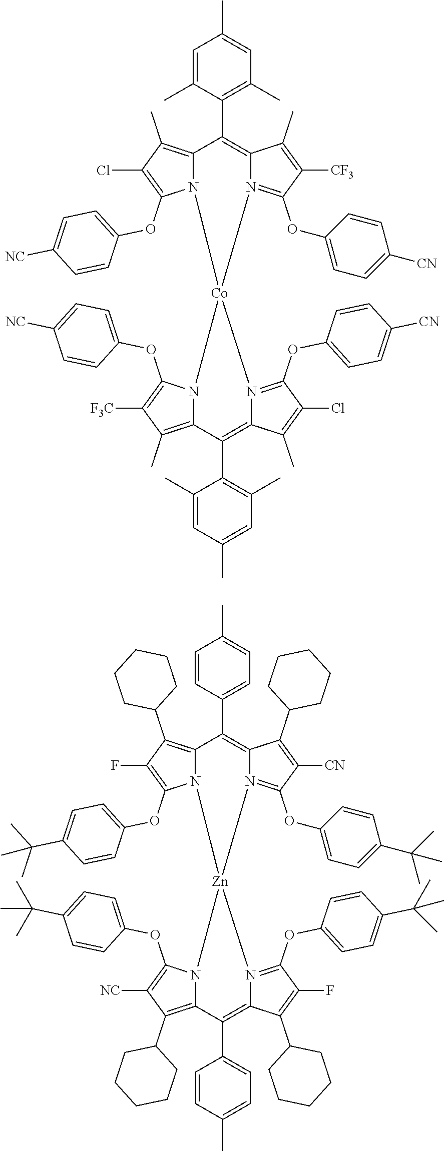

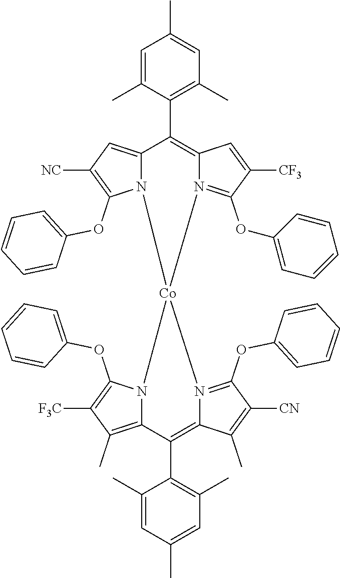

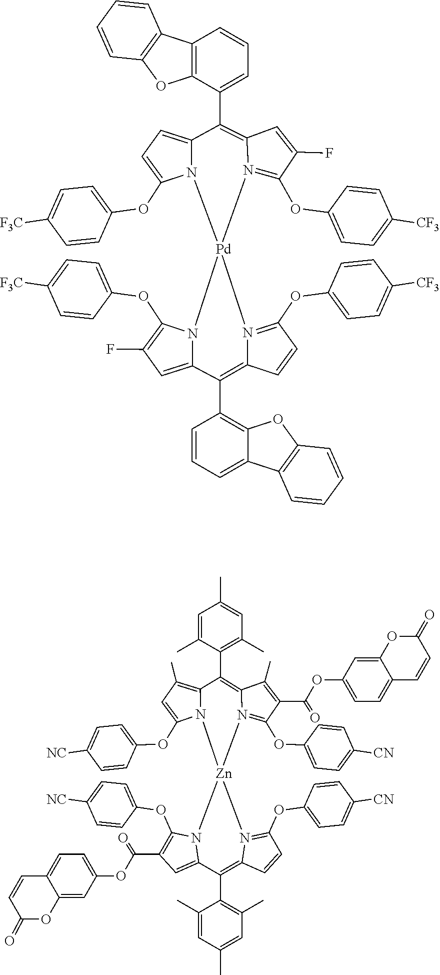

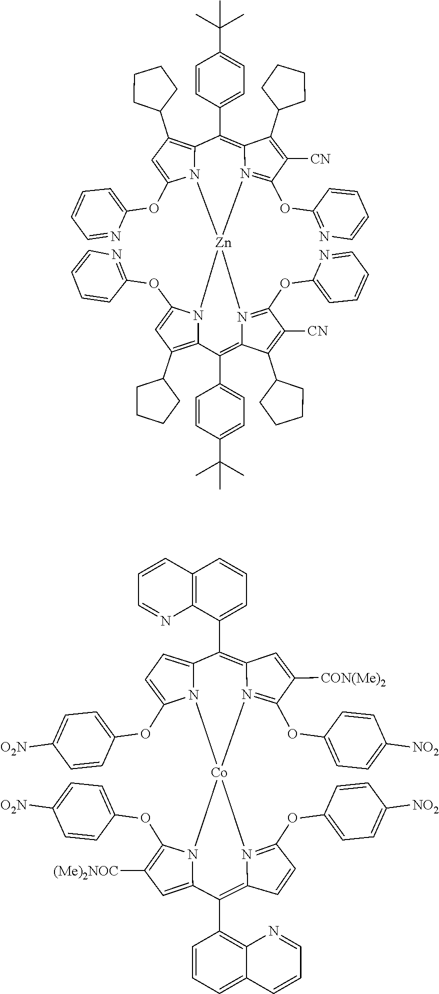

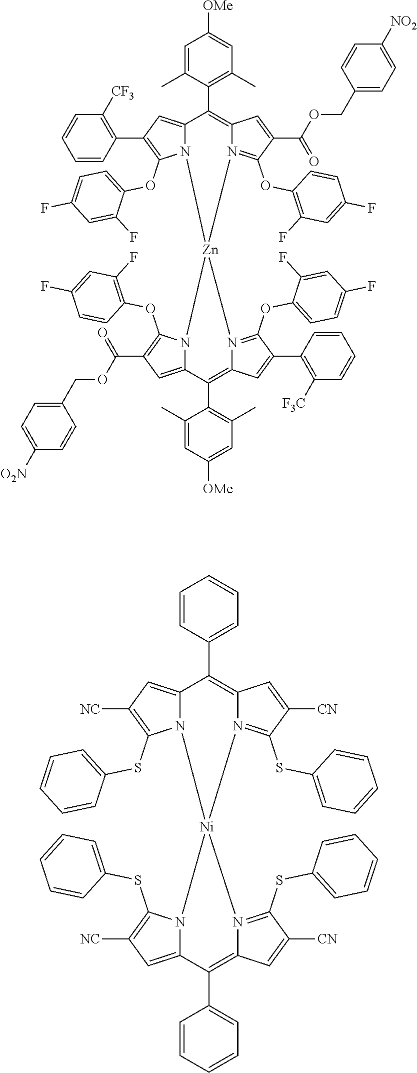

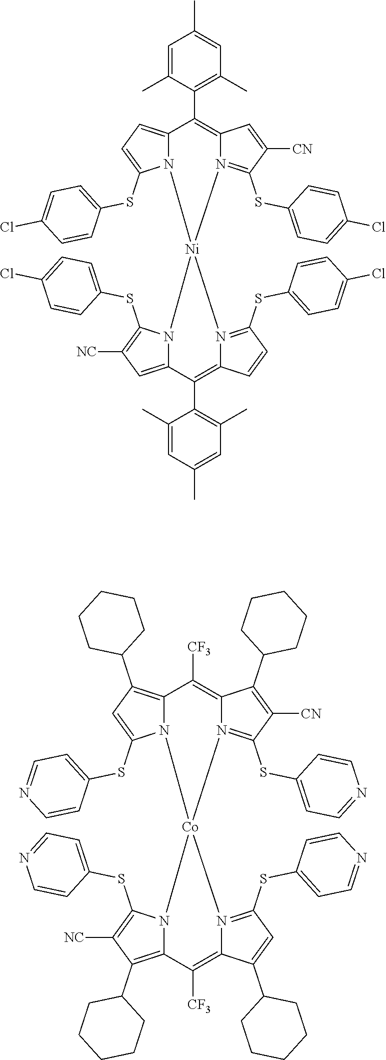

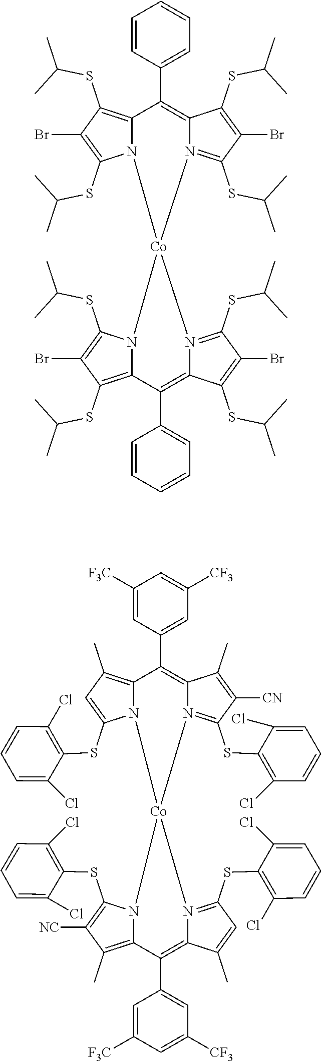

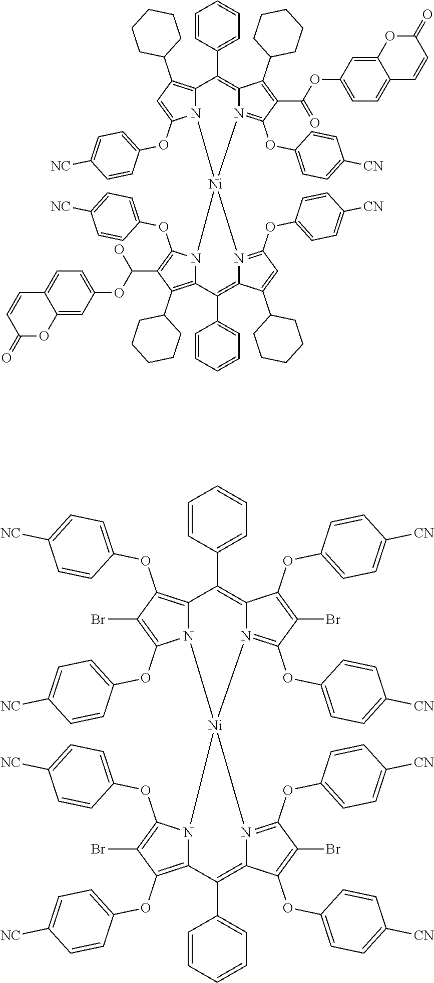

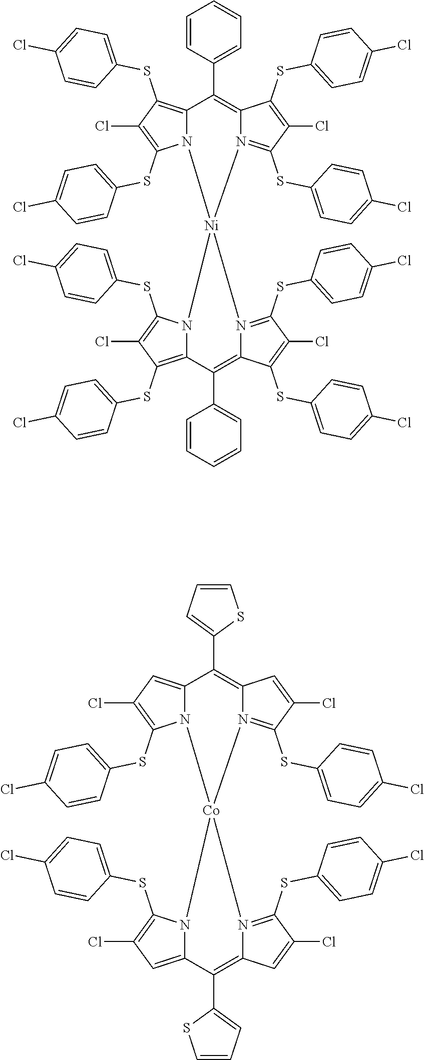

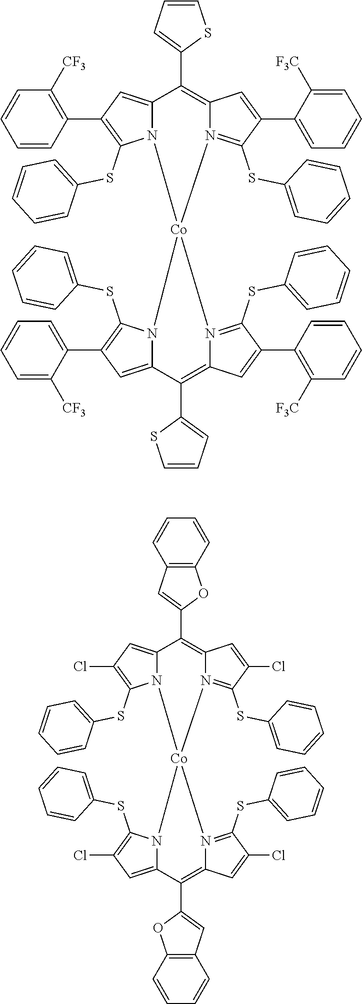

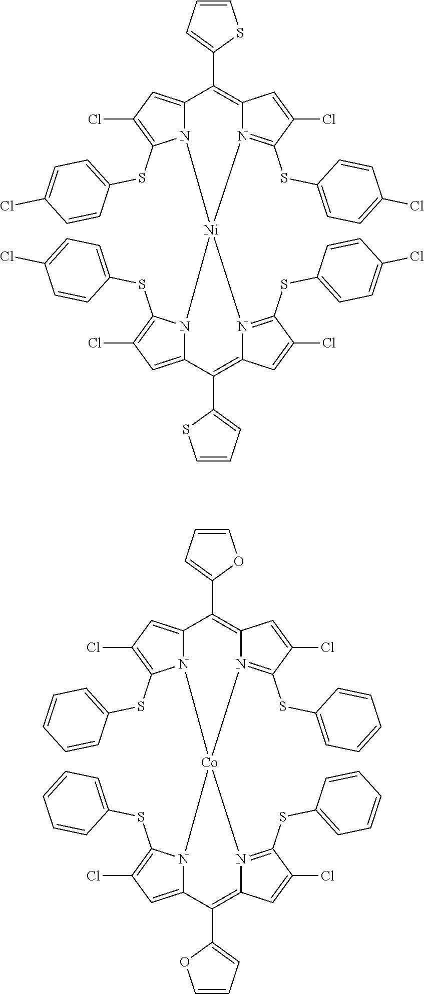

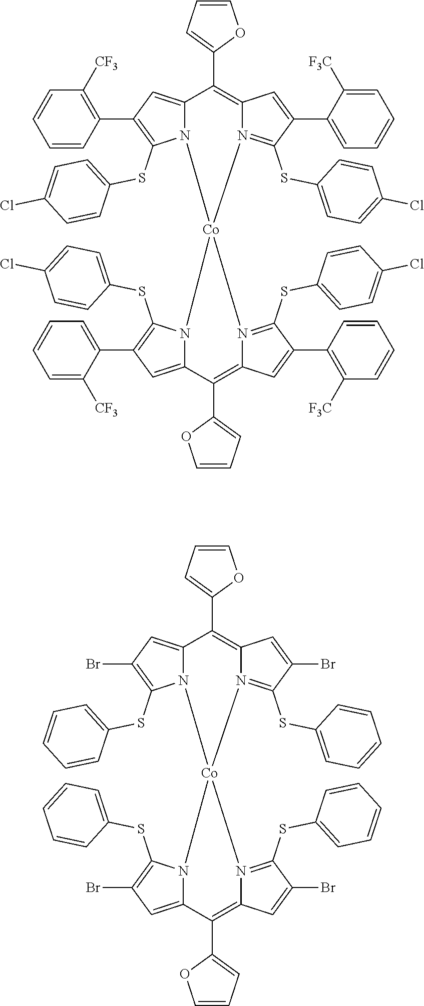

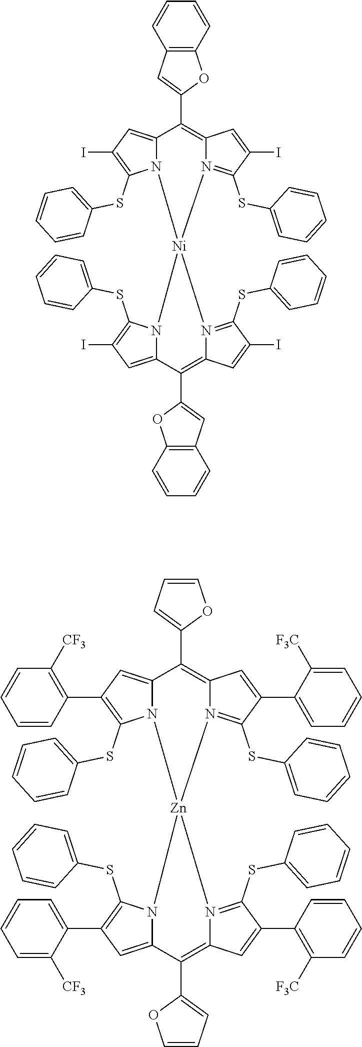

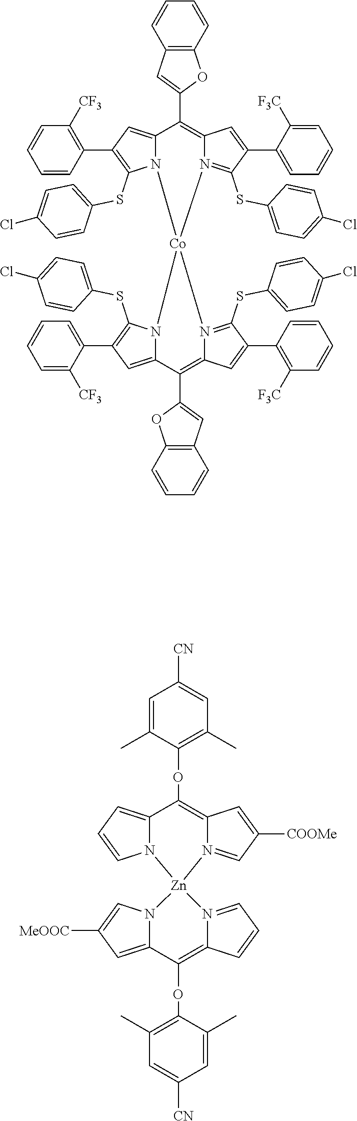

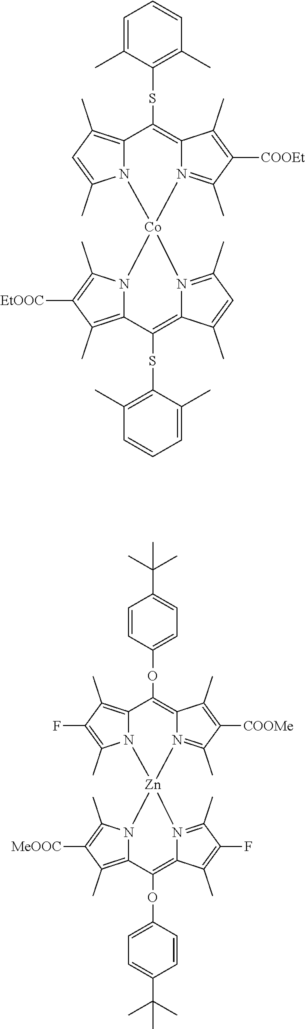

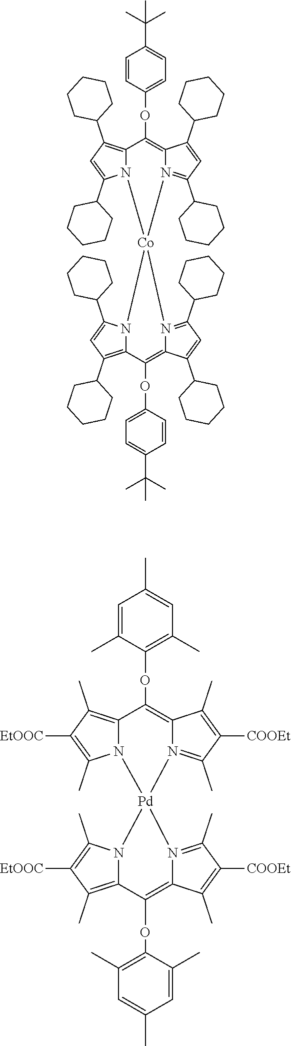

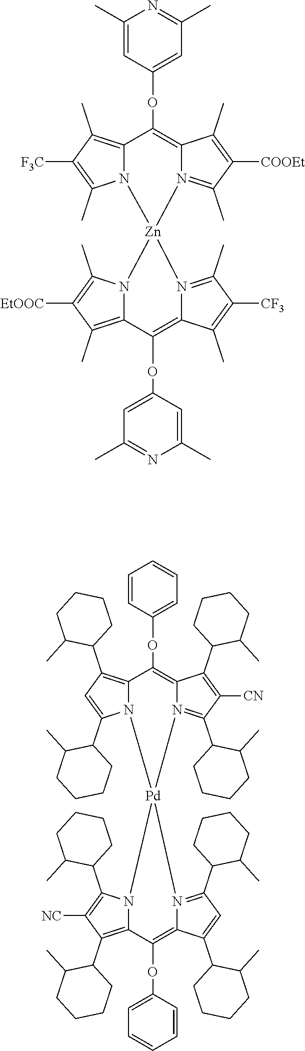

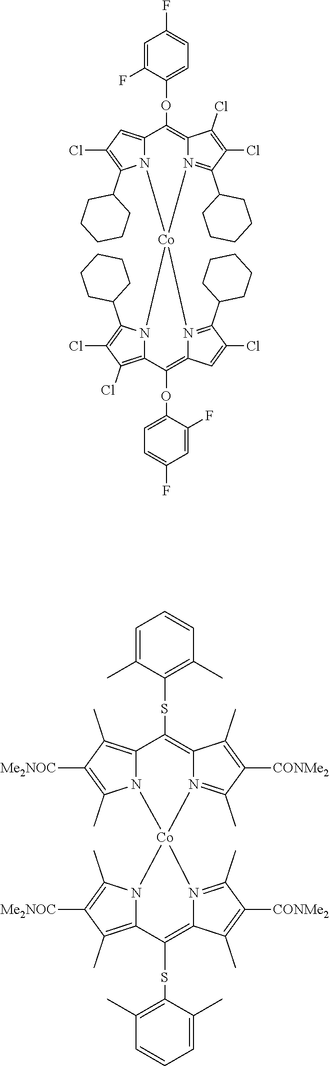

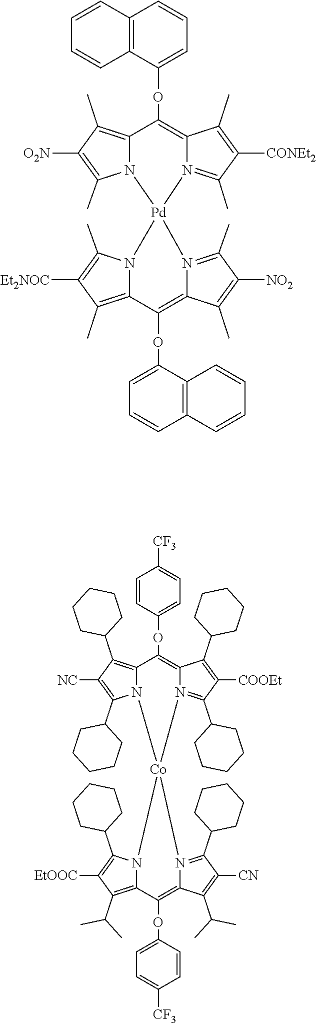

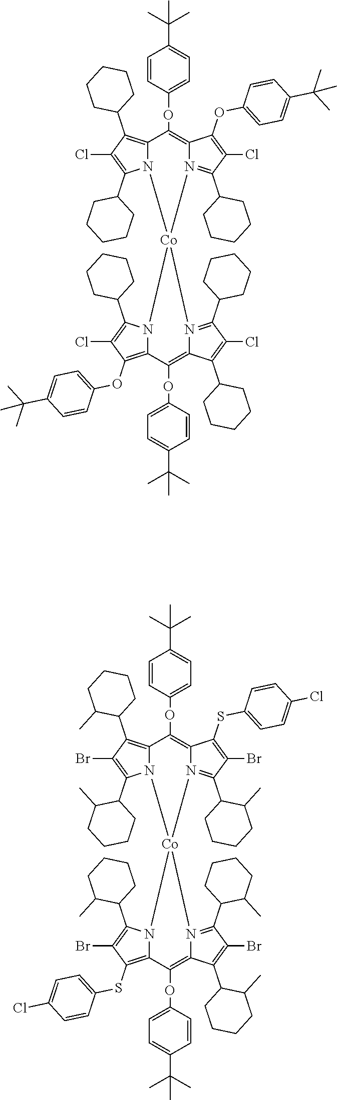

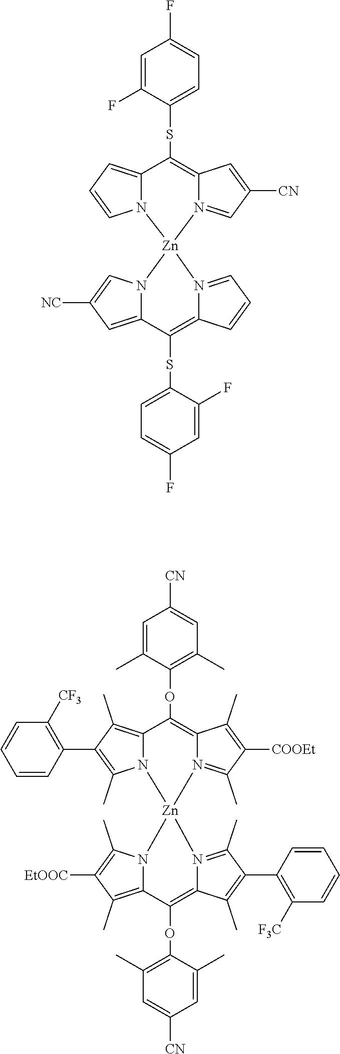

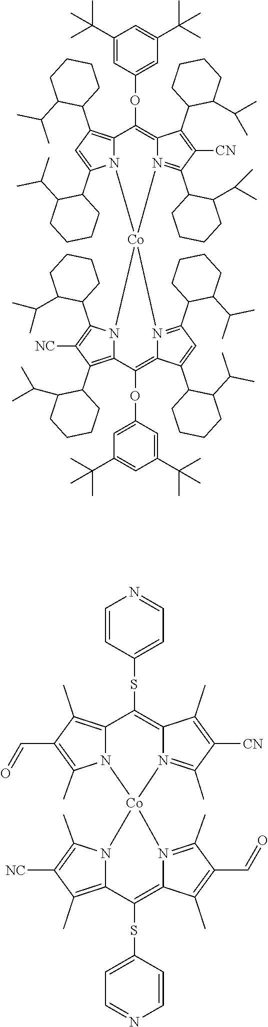

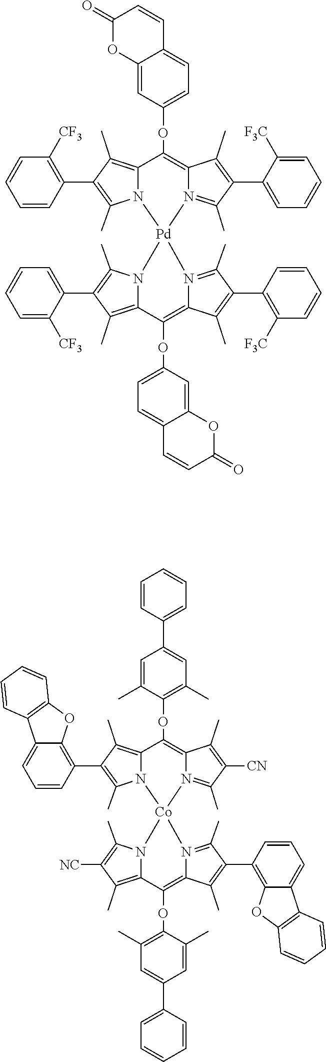

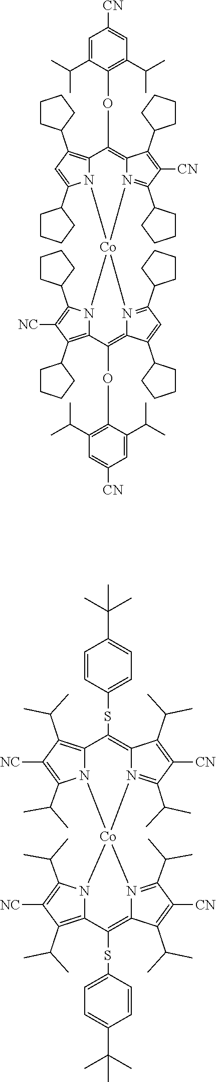

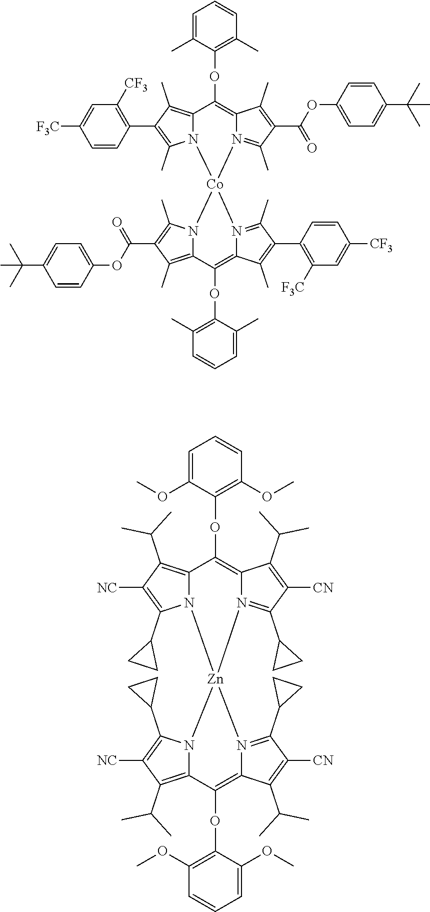

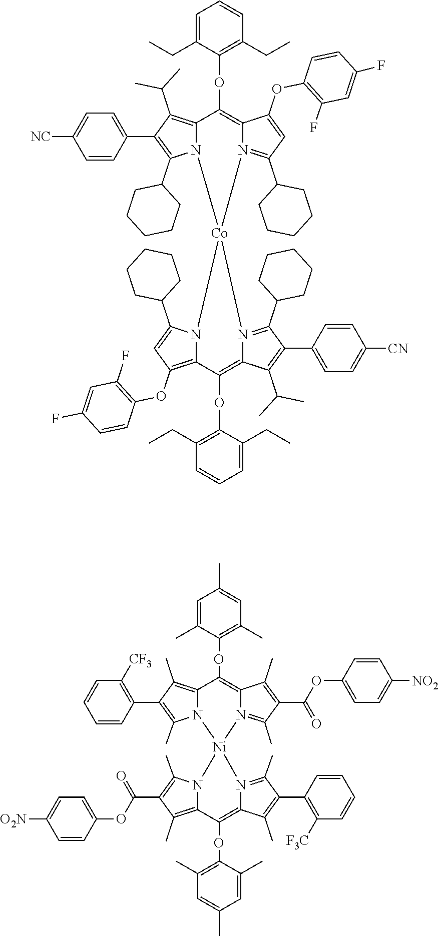

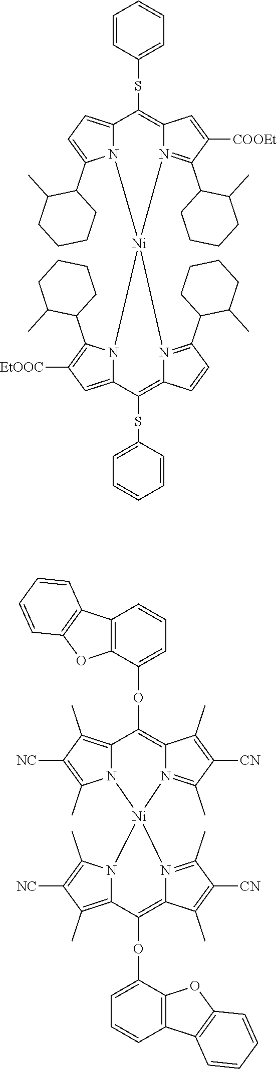

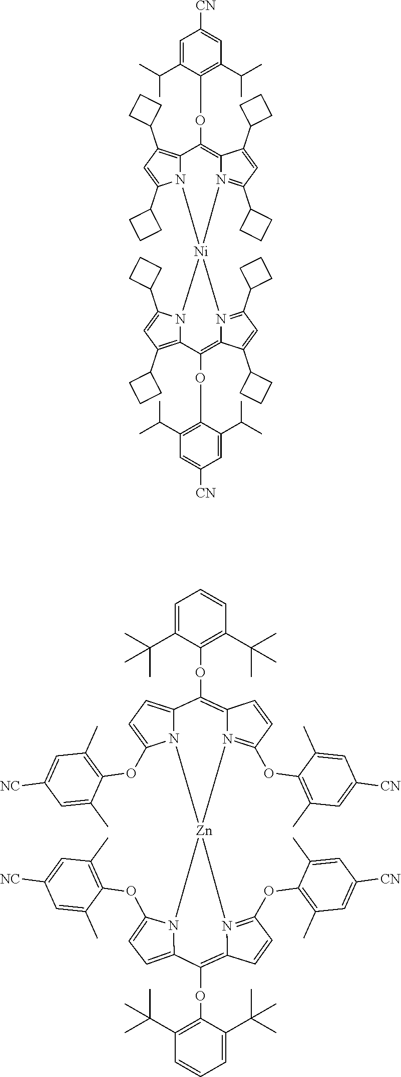

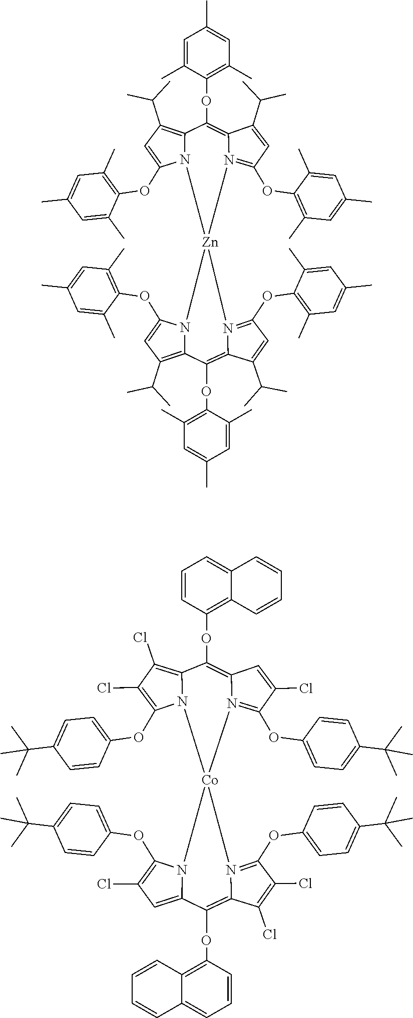

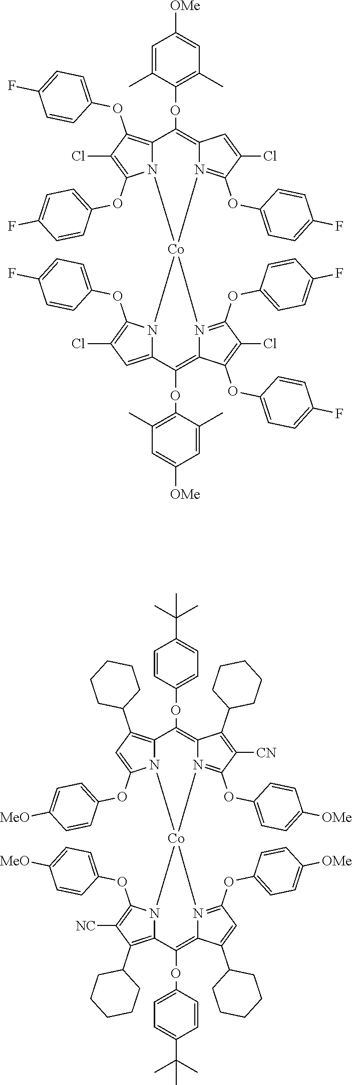

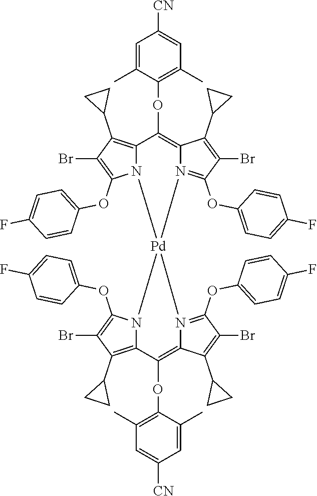

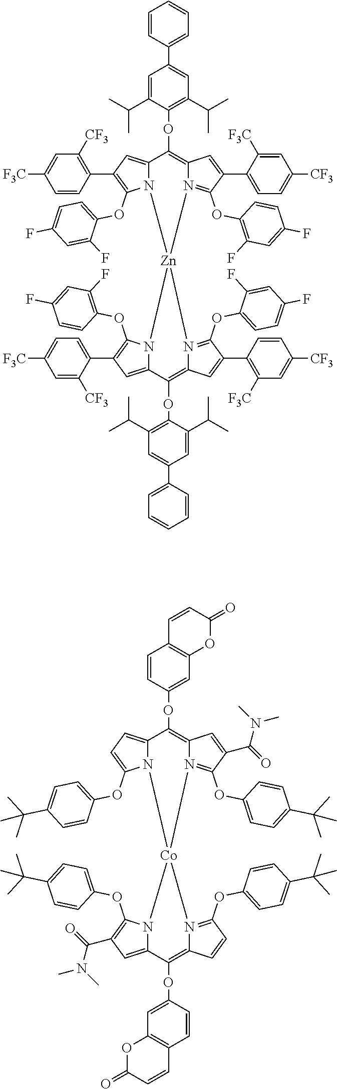

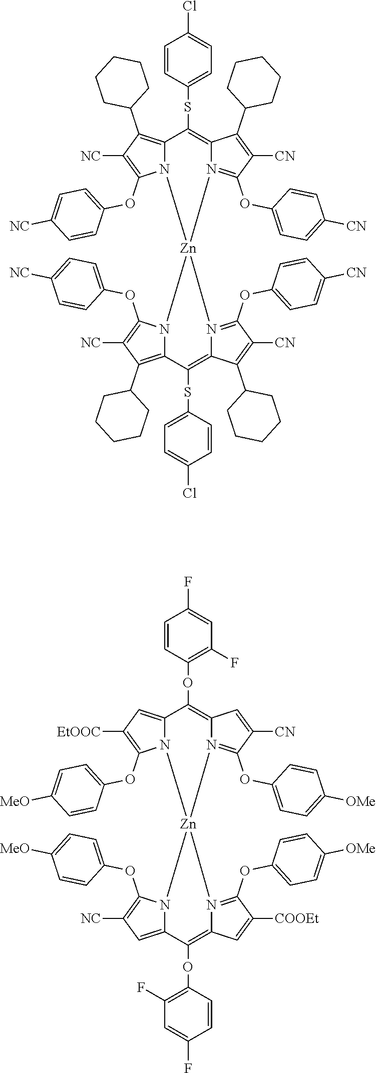

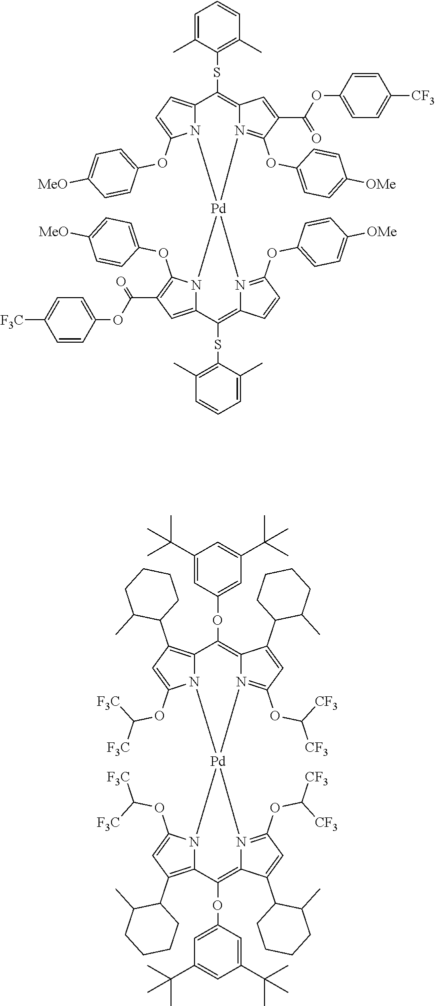

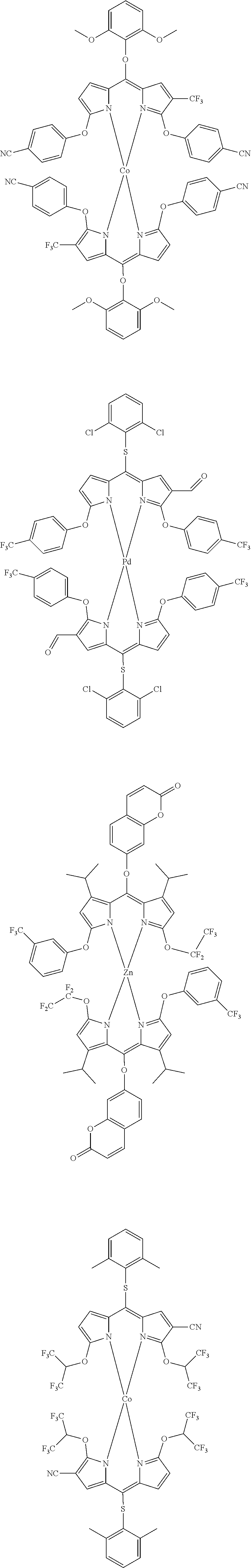

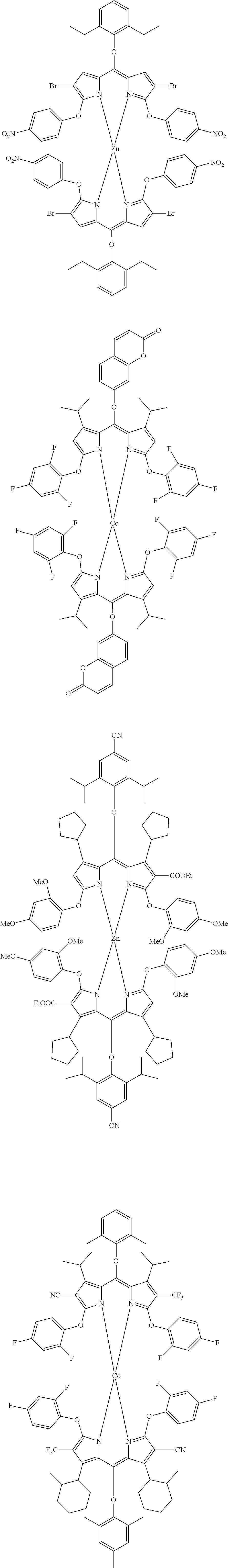

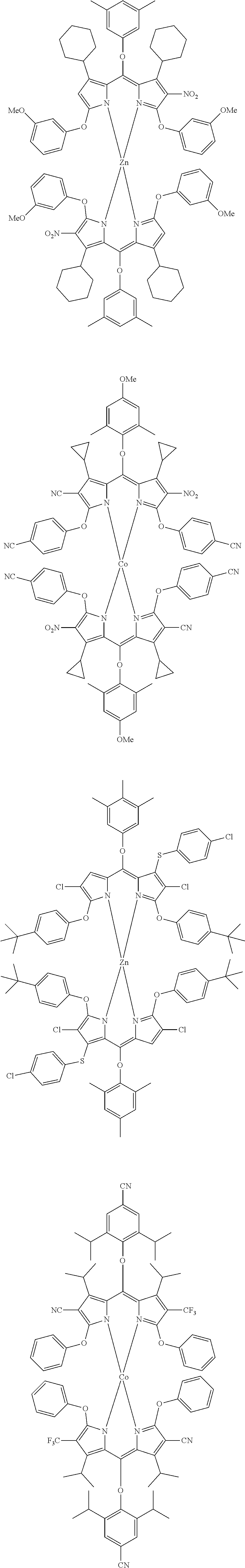

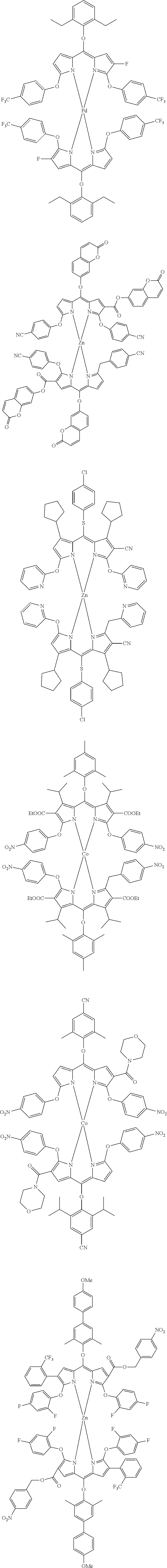

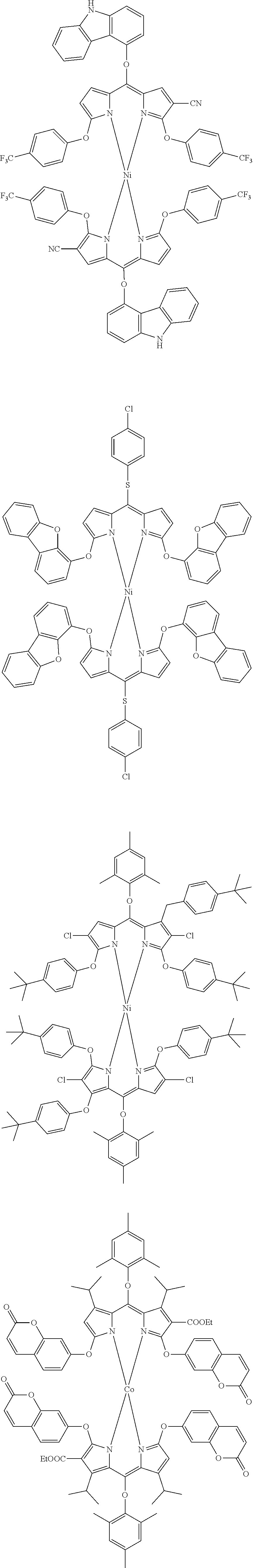

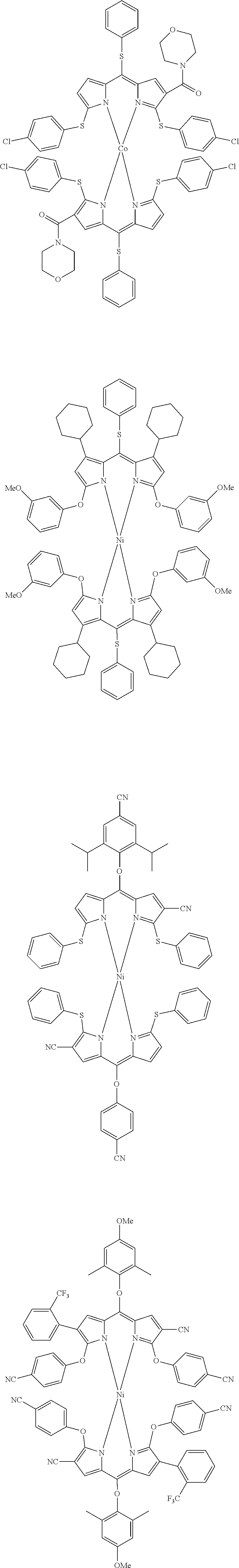

In the compounds,

Me means a methyl group, and Et means an ethyl group.

In one embodiment of the present specification, the composition for forming an optical film further includes at least one selected from the group consisting of an antioxidant, an antistatic agent, a hindered amine-based light stabilizer, a UV stabilizer, a crosslinking agent, a coupling agent, a catalyst and a solvent. Preferably, the composition for forming an optical film further includes an antioxidant, an antistatic agent and a hindered amine-based light stabilizer.

In one embodiment of the present specification, examples of the antioxidant may include KINOX 10 (pentaerythrityl tetrakis[3-(3,5-di-tert-butyl-4-hydroxyphenyl)propionate]), KINOX 76 (octadecyl-3-(3,5-di-tert-butyl-4-hydroxyphenyl)propionate), KINOX 30 (1,3,5-trimethyl-2,4,6-tris(3,5-di-tert-butyl-4-hydroxybenzyl)benzene), KINOX 34 (1,3,5-tris(3,5-di-tert-butyl-4-hydroxybenzyl)-1,3,5-triazine-2,4,6-(1H,3H,5H)-trione), KINOX 98 (N,N′-hexamethylene-bis[3-(3,5-di-tert-butyl-4-hydroxyphenyl)propionamide]), KINOX 68 (tris(2,4-di-tert-butylphenyl)phosphite), KINOX 28 (bis (2,4-dicumylphenyl)pentaerythritol diphosphite), or combinations thereof, however, the antioxidant is not limited thereto, and those commonly used in the art may be employed.

In one embodiment of the present specification, as the antistatic agent, ionic compounds or metal salts may be used, for example. As the ionic compound, known organic salts usable as an antistatic agent may be used, for example. As the metal salt, known alkali metal salts or alkaline earth metal salts usable as an antistatic agent may be used, for example. When further including the antistatic agent, the adhesive layer has antistatic properties and thereby may reduce generation of static electricity.

In one embodiment of the present specification, examples of the hindered amine-based light stabilizer may include LA 63P (1,2,3,4-butanetetracarboxylic acid-β,β,β′,β′-tetramethyl-2,4,8,10-tetraoxaspiro[5.5]undecane-3,9-diethanol copolymer, 1,2,2,6,6-pentamethyl-4-piperidinyl ester), IRESORB 770 (decanedioic acid, 1,10-bis(2,2,6,6-tetramethyl-4-piperidinyl)ester), Tinuvin 292 (decanedioic acid, 1,10-bis(1,2,2,6,6-pentamethyl-4-piperidinyl)ester), Tinuvin 123 (decanedioic acid, bis (2,2,6,6-tetramethyl-1-(octyloxy)-4-piperidinyl)ester), or combinations thereof, however, the hindered amine-based light stabilizer is not limited thereto, and those commonly used in the art may be employed.

Examples of the UV stabilizer may include LA-F 70 (tris[2-hydroxy-3-methyl-4-hexyloxyphenyl]-1,3,5-triazine), Tinuvin 360 (bis[2-hydroxy-5-tert-octyl-3-(benzotriazol-2-yl)phenyl]methane), Tinuvin 99-2 (benzenepropanoic acid, 3-(2H-benzotriazol-2-yl)-5-(1,1-dimethylethyl)-4-hydroxy-C7-9-branched and linear alkyl esters), Tinuvin 1130 (a) 50% 13-[3-(2-H-benzotriazol-2-yl)-4-hydroxy-5-tert-butylphenyl]-propionic acid-poly(ethylene glycol) 300-ester, b) 38% bis{-[3-(2-H-benzotriazol-2-yl)-4-hydroxy-5-tert-butylphenyl]-propionic acid}-poly(ethylene glycol) 300-ester, and c) 12% polyethylene glycol), Tinuvin 400 (2-[4-[(2-hydroxy-3-dodecyloxypropyl)oxy]-2-hydroxyphenyl]-4,6-bis(2,4-dimethylphenyl)-1,3,5-triazine & 2-[4-[(2-hydroxy-3-tridecyloxypropyl)oxy]-2-hydroxyphenyl]-4,6-bis(2,4-dimethylphenyl)-1,3,5-triazine), Tinuvin 477, or combinations thereof, however, the UV stabilizer is not limited thereto, and those commonly used in the art may be employed.

The crosslinking agent may be an isocyanate-based crosslinking agent.

Examples of the isocyanate-based crosslinking agent may include tolylene diisocyanate, xylene diisocyanate, 2,4-diphenylmethane diisocyanate, 4,4-diphenylmethane diisocyanate, hexamethylene diisocyanate, isophorone diisocyanate, tetramethylxylene diisocyanate, naphthalene diisocyanate, triphenylmethane triisocyanate, methylenebis triisocyanate and the like, but are not limited thereto.

The coupling agent may be a silane-based coupling agent, but is not limited thereto, and those known in the art may be properly employed.

The catalyst may be a tin-based catalyst, but is not limited thereto, and those known in the art may be properly employed.

The solvent may be methyl ethyl ketone, but is not limited thereto, and those known in the art may be properly employed.

In one embodiment of the present specification, the composition for forming an optical film includes a styrene-based resin or elastomer, an urethane-based resin or elastomer, a polyolefin-based resin or elastomer, a polyoxyalkylene-based resin or elastomer, a polyester-based resin or elastomer, a polyvinyl chloride-based resin or elastomer, a polycarbonate-based resin or elastomer, a polyphenylene sulfide-based resin or elastomer, a polyamide-based resin or elastomer, an acrylate-based resin or elastomer, an epoxy-based resin or elastomer, a silicone-based resin or elastomer, a fluorine-based resin or elastomer, or a mixture thereof.

In one embodiment of the present specification, the composition for forming an optical film further includes an azo-based dye or pigment, a metal-containing azo-based dye or pigment, a quinoline-based dye or pigment, a methine-based dye or pigment, a coumarin-based dye or pigment, a porphyrin-based dye or pigment, an azaporphyrin-based dye or pigment, a phthalocyanine-based dye or pigment, an anthraquinone-based dye or pigment, a perylene-based dye or pigment, a squarylium-based dye or pigment, a benzoazole-based dye or pigment, or a triazine-based dye or pigment.

In one embodiment of the present specification, the composition for forming an optical film includes the compound represented by Chemical Formula 1 in 0.001 parts by weight to 5 parts by weight and the binder resin in 85 parts by weight to 99 parts by weight based on the total weight of 100 parts by weight of the composition for forming an optical film.

The composition for forming an optical film may further include, as necessary, the antioxidant in 0.01 parts by weight to 5 parts by weight, the antistatic agent in 0.01 parts by weight to 5 parts by weight, the hindered amine-based light stabilizer in 0.001 parts by weight to 1 parts by weight, the crosslinking agent and the coupling agent in 0.001 parts by weight to 1 parts by weight, and the catalyst in 0.0001 parts by weight to 0.1 parts by weight based on the total weight of 100 parts by weight of the composition for forming an optical film.

The composition for forming an optical film may further include, as necessary, the solvent in parts by weight to 50 parts by weight based on the total weight of 100 parts by weight of the composition for forming an optical film.

The composition for forming an optical film may further include, as necessary, the UV stabilizer in 0.05 parts by weight to 5 parts by weight based on the total weight of 100 parts by weight of the composition for forming an optical film.

The composition for forming an optical film may further include, as necessary, the dye or pigment in 0.001 parts by weight to 5 parts by weight based on the total weight of 100 parts by weight of the composition for forming an optical film.

The content of the dye may be adjusted in order to obtain an optical film having target transmittance according to the intended use, and when using in less than 0.001 parts by weight, the effect of using the dye is difficult to obtain. It is preferred not to exceed a maximum of 5 parts by weight, and this is due to the fact that precipitates of the dye may occur in the adhesive when the content of the dye is too excessive.

The crosslinking agent and the coupling agent are materials performing crosslinking of the adhesive, and the effect is difficult to obtain when the content is less than 0.001 parts by weight, and the content of greater than 1 parts by weight may cause direct damages by reacting with the dye.

The catalyst is a material facilitating the crosslinking reaction, and the effect is difficult to obtain when the content is less than 0.0001 parts by weight, and the content of greater than 0.1 parts by weight may decline adhesive properties.

The antioxidant is a material preventing oxidation of the composition, and the effect is difficult to obtain when the content is less than 0.01 parts by weight, and the content of greater than 5 parts by weight may cause a reliability problem of the dye or decline adhesive properties of the adhesive.

The antistatic agent is a material performing a role of preventing static electricity of the film, and the effect is difficult to obtain when the content is less than 0.01 parts by weight, and the content of greater than 5 parts by weight may cause a reliability problem of the dye or decline adhesive properties of the adhesive.

The hindered amine-based light stabilizer is a material enhancing light resistance reliability, and the effect is difficult to obtain when the content is less than 0.001 parts by weight, and the content of greater than 1 parts by weight may decline adhesive properties.

When a film such as an anti-reflection layer is not used in the optical film according to the present specification, the UV stabilizer may be added to enhance UV reliability. The effect of the UV stabilizer is difficult to obtain when the content is less than 0.05 parts by weight, and the content of greater than 5 parts by weight may decline adhesive properties.

One embodiment of the present specification provides an optical film including the composition for forming an optical film or a cured material thereof.

In one embodiment of the present specification, the optical film may include the composition for forming an optical film according to one embodiment of the present specification as it is.

In one embodiment of the present specification, the optical film may include a cured material of the composition for forming an optical film according to one embodiment of the present specification.

In the present specification, the cured material is obtained by curing after the solvent included in the adhesive composition is dried, and each constituent included in the adhesive composition is crosslinked by forming chemical and/or physical bonds.

In one embodiment of the present specification, the optical film is an adhesive film or a binder resin film.

The optical film may preferably be an adhesive film. When the optical film is an adhesive film, the adhesive film may function as an optical adhesive layer. The function as an optical adhesive layer means forming a black color adhesive film incorporating an organic dye capable of absorbing visible light, and an OLED panel including the same suppresses high panel reflectance. In other words, visible light transmittance of the adhesive film may be controlled in a range of approximately 30% to 90%, and transmittance in a visible region may be properly adjusted depending on the panel reflectance and the reflected color.

When the optical film is an adhesive film, the binder resin included in the composition for forming an optical film may specifically be an adhesive resin, that is, an acrylate-based resin or elastomer, and as constituents other than the binder resin included in the composition for forming an optical film, the constituents described above may be used.

As the acrylate-based resin or elastomer, a copolymer formed with two or more types of monomers selected from among, for example, methyl (meth)acrylate, ethyl (meth)acrylate, butyl (meth)acrylate, ethylhexyl (meth)acrylate, hydroxyethyl (meth)acrylate, (meth)acrylic acid and dimethylaminoethyl (meth) acrylate may be used, however, the acrylate-based resin or elastomer is not limited thereto. When the optical film is an adhesive film, the binder resin film separately included in the adhesive optical filter is not particularly limited, and materials and thicknesses used in the art may be employed. In examples to describe later, a binder resin film-attached optical film is described, however, the binder resin film does not affect reliability evaluation of heat resistance, moisture resistance and light resistance to describe later. As the adhesive optical filter, descriptions to provide later are applied.

In one embodiment of the present application, the adhesive film may have a thickness of greater than or equal to 3 μm and less than or equal to 100 μm. The thickness may be preferably greater than or equal to 5 m and less than or equal to 80 μm, and more preferably greater than or equal to 10 μm and less than or equal to 50 μm.

In one embodiment of the present specification, the adhesive film further includes a release layer provided on one surface thereof.

FIG. 6 illustrates a structure when a release layer (4) is provided on one surface of the adhesive film (3) according to one embodiment of the present specification.

In the present specification, the release layer means a transparent layer formed on one surface of the adhesive film through a release treatment, and, as long as it does not adversely affect in the manufacturing process of the adhesive film, may be employed without limit in terms of materials, thicknesses, properties and the like. The release layer provided on one surface of the adhesive film may be removed after manufacturing the adhesive film.

The release layer may include one or more selected from the group consisting of acetate-based, polyester-based, polyethersulphone-based, polycarbonate-based, polyamide-based, polyimide-based, polyolefin-based, cycloolefin-based, polyurethane-based, acryl-based, fluorine-based and silicone-based resins, but is not limited thereto.

The release layer may have a thickness of greater than or equal to 10 nm and less than or equal to 1,000 nm, preferably greater than or equal to 20 nm and less than or equal to 800 nm, and more preferably greater than or equal to 40 nm and less than or equal to 100 nm, however, the thickness is not limited thereto.

In the present specification, the adhesive film may be manufactured by coating the adhesive composition described above on the release layer or a base using a bar coater. The adhesive film may be manufactured by coating the adhesive composition described above on a base using a bar coater, and then drying the result. Descriptions on the base will be provided later. The methods of coating and drying are not particularly limited, and methods used in the art may be properly employed.

In addition, in another embodiment of the present specification, the optical film may be a binder resin film.

When the optical film is a binder resin film, the binder resin included in the composition for forming an optical film may specifically be polymethyl methacrylate, polystyrene, polyethylene, polyethylene terephthalate or the like, and as constituents other than the binder resin, the constituents described above may be used.

In one embodiment of the present specification, the binder resin film may have a thickness of greater than or equal to 10 μm and less than or equal to 200 μm, preferably greater than or equal to 15 μm and less than or equal to 100 μm, and more preferably greater than or equal to 20 μm and less than or equal to 75 μm.

In one embodiment of the present specification, TAC (cellulose triacetate) may be used as the binder resin layer, however, the binder resin layer is not limited thereto.

In another embodiment of the present specification, the optical film may further include a surface treatment layer.

The surface treatment layer may be, for example, an anti-reflection layer or an anti-glare layer. The surface treatment layer may include a low refractive index layer having a refractive index of approximately 1.4 or less for a wavelength of 550 nm. A lower limit of the refractive index of the low refractive index layer may be, for example, approximately 1.2, and a specific refractive index range may be from 1.31 to 1.35. The low refractive index layer may include a photocurable acrylate containing hollow silica and a fluorine-based acrylate. The surface treatment layer may further include a hard coating layer on one surface of the low refractive index layer. The hard coating layer is a layer protecting a base surface from being damaged by friction and the like, and includes an organic coating agent such as melamine, acryl or urethane, an inorganic coating agent such as silicon-based, or an organic-inorganic hybrid coating agent, and normally has a thickness of 5 m to 30 m.

A material and a thickness of the surface treatment layer are not particularly limited, and materials and thicknesses commonly used in the art may be employed.

The surface treatment layer may be provided on one surface of the binder resin film.

One embodiment of the present specification provides an adhesive optical filter including the optical film.

Hereinafter, an adhesive optical filter when the optical film is an adhesive film will be described.

One embodiment of the present specification provides an adhesive optical filter including an optical film; and a surface treatment layer.

The adhesive optical filter includes an adhesive film; and a surface treatment layer provided on one surface of the adhesive film.

In addition, one embodiment of the present specification provides an adhesive optical filter further including a binder resin film between the adhesive film and the surface treatment layer.

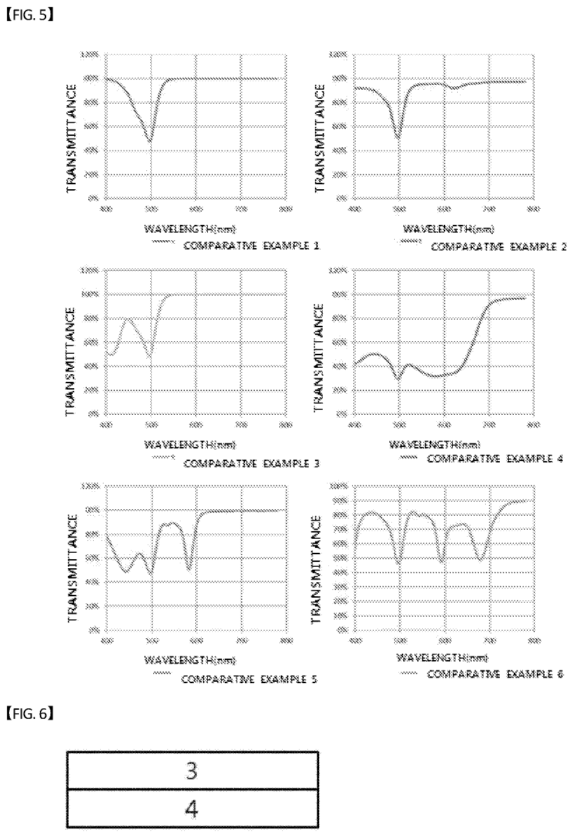

Specifically, FIG. 7 illustrates a structure of the adhesive optical filter according to one embodiment of the present specification. The adhesive optical filter (10) includes a binder resin film (2); the adhesive film (3) provided on one surface of the binder resin film (2); and a surface treatment layer (1) provided on a surface opposite to the surface where the binder resin film (2) and the adhesive film (3) are in contact with each other.

In one embodiment of the present specification, the adhesive film or the binder resin film may each be a single layer or a multilayer. The multilayer means 2 or 3 layers.

When the adhesive film or the binder resin film is a multilayer, the adhesive films or the binder resin films may be each independently the same as or different from each other. Being different from each other may be a difference in the types of the constituents included each film, or may be a difference in the content thereof.

The adhesive optical filter may be manufactured by consecutively laminating a surface treatment layer provided on one surface of a binder resin film, and then laminating the adhesive film on a surface opposite to the surface in contact with the surface treatment layer of the binder resin film.

In addition, the adhesive optical filter may be manufactured by laminating a surface treatment layer provided on one surface of a base, preparing the adhesive film separately, and then attaching the adhesive film on a surface opposite to the surface in contact with the surface treatment layer laminated on the base.

The method of laminating the surface treatment layer on one surface of the base and the method of laminating the adhesive film on a surface opposite to the surface in contact with the surface treatment layer of the binder resin film are not particularly limited, and, for example, methods such as coating may be employed, and other methods used in the art may be properly employed.

One embodiment of the present specification provides a display device including the optical film described above. One embodiment of the present specification provides a display device including an adhesive optical filter including the optical film.

One embodiment of the present specification provides a display device including the adhesive optical filter described above.

In one embodiment of the present specification, the display device further includes a display panel, and the optical film is provided on the display panel.

In one embodiment of the present specification, the display panel is a spontaneous light emitting display panel or a non-spontaneous light emitting display panel.

Examples of the spontaneous light emitting display panel may include an OLED panel and the like not requiring a backlight, and examples of the non-spontaneous light emitting display panel may include an LCD panel and the like requiring a backlight.

In one embodiment of the present specification, the display panel is an OLED panel, and the display device is an OLED device including an OLED panel; and the optical film provided on one surface of the OLED panel.

In one embodiment of the present specification, the OLED panel further includes a white pixel.

In one embodiment of the present specification, the OLED panel is a white OLED panel.

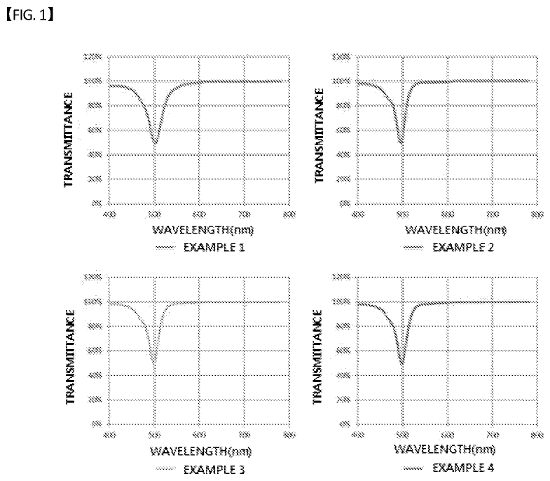

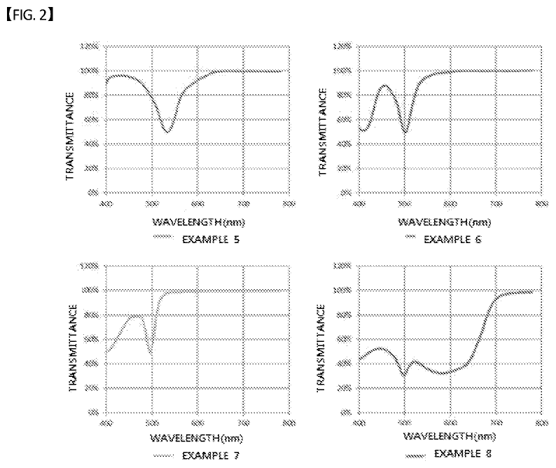

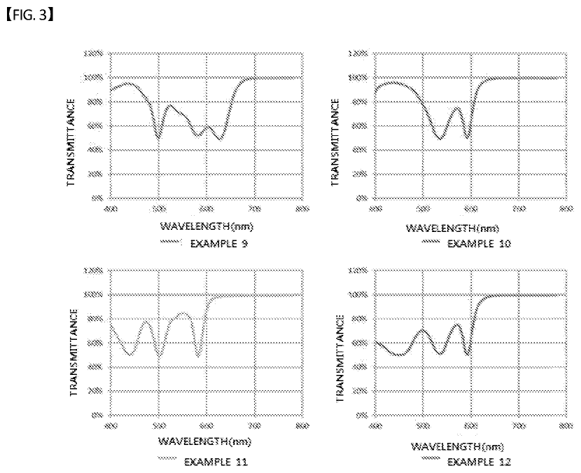

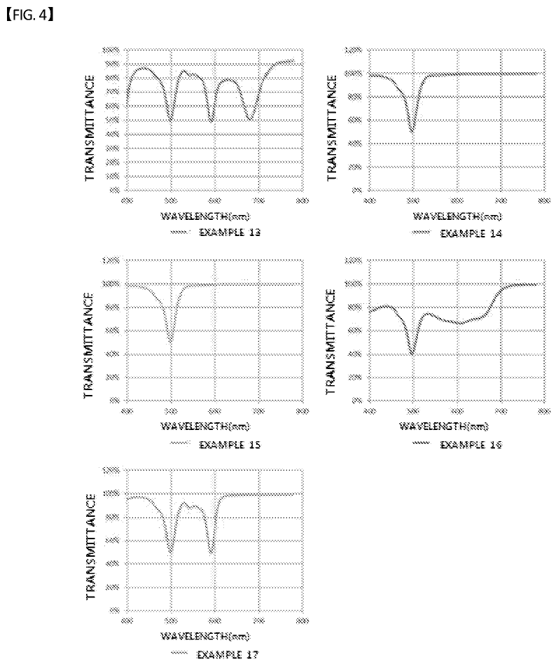

In one embodiment of the present specification, the display device may be included in, for example, TVs, computer monitors, laptops, mobile phones and the like. FIG. 1 to FIG. 4 are diagrams showing wavelength-dependent absorption spectra of the optical films according to examples and FIG. 5 is a diagram showing wavelength-dependent absorption spectra of the optical films according to comparative examples, and the wavelength-dependent absorption spectra of the optical films may be measured using a UV-vis spectrometer. In the diagrams of FIG. 1 to FIG. 5, the horizontal axis represents a wavelength (nm) of the optical film, and the vertical axis represents absorbance. In the comparative examples of FIG. 5, other dyes absorbing a 500 nm region have low reliability.

FIG. 8 illustrates a structure of an OLED device (30), one example of the display device according to one embodiment of the present specification. The OLED device (30) of the present specification may include an OLED panel (20) and an adhesive optical filter (10) provided on one surface of the OLED panel (20) and having the adhesive film (3), the binder resin film (2) and the surface treatment layer (1) consecutively formed therein. Specifically, in the OLED device (30), one surface where the OLED panel (20) and the optical filter (10) are in contact with each other is a surface opposite to the surface where the adhesive film (3) and the binder resin film (2) are in contact with each other.

In the OLED device, the descriptions provided above are applied to the optical film.

In the present specification, the OLED panel may consecutively include a substrate, a lower electrode, an organic material layer and an upper electrode. The organic material layer may include an organic material capable of emitting light when a voltage is applied to the lower electrode and the upper electrode. Any one of the lower electrode and the upper electrode may be an anode, and the other one may be a cathode. The anode is an electrode where holes are injected, and may be made with conductive materials having high work function. The cathode is an electrode where electrons are injected, and may be made with conductive materials having low work function. As the anode, a transparent metal oxide layer such as ITO (indium tin oxide) or IZO (indium zinc oxide) having high work function may be commonly used, and as the cathode, a metal electrode having low work function may be used. An organic material layer is generally transparent, and a transparent display may be obtained when the upper electrode and the lower electrode are made to be transparent. In one example, a transparent display may be obtained when the thickness of the upper electrode or the lower electrode is employed to be very thin.

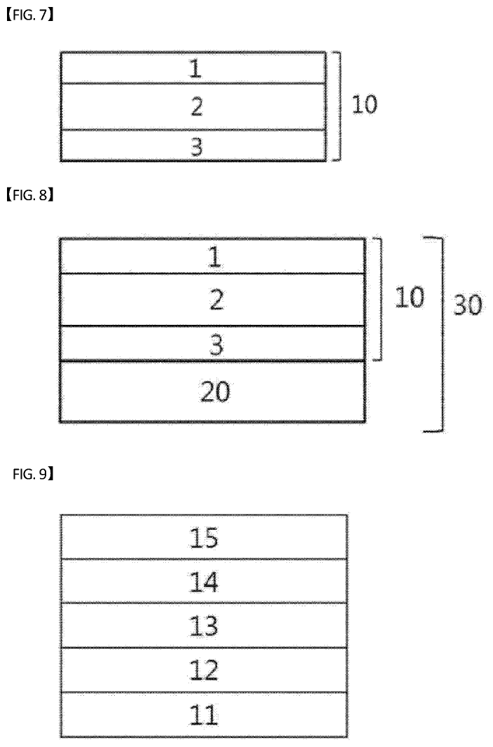

FIG. 9 illustrates of a structure of the OLED panel according to one embodiment of the present specification, and it may be identified that the OLED panel consecutively includes a substrate (11); a lower electrode (12); an organic material layer (13); and an upper electrode (14). The OLED panel may further include an encapsulation substrate (15), which functions to prevent inflow of moisture and/or oxygen from the outside, on the upper electrode.

The organic material layer may include a light emitting layer, and may further include a common layer for charge injection and transport. Specifically, the common layer for charge injection and transport may include a hole transporting layer, a hole injecting layer, an electron injecting layer and an electron transporting layer for balancing electrons and holes, but is not limited thereto.

The optical film may be disposed on a side of the OLED panel where light emits. For example, the adhesive optical filter may be disposed on an outer side of the substrate in a bottom emission structure where light emits toward the substrate side, and the adhesive optical filter may be disposed on an outer side of the encapsulation substrate in a top emission structure where light emits toward the encapsulation substrate side.

Specifically, (a) of FIG. 10 illustrates the OLED device when the OLED panel (20) has a bottom emission structure, and in the bottom emission structure side where light emits from the organic material layer (13) toward the substrate (11) side, the adhesive optical filter (10) may be provided on a surface opposite to the surface where the substrate (11) and the lower electrode (12) are in contact with other, and a surface opposite to the surface in contact with the binder resin film (2) of the adhesive film (3) included in the adhesive optical filter (10) is provided in contact with the substrate (11) of the OLED panel (20).

(b) of FIG. 10 illustrates the OLED device when the OLED panel (20) has a top emission structure, and in the top emission structure where light emits from the organic material layer (13) toward the encapsulation substrate (15) side, the adhesive optical filter (10) may be provided on a surface opposite to the surface where the encapsulation substrate (15) and the upper electrode (14) are in contact with each other, and a surface opposite to the surface in contact with the binder resin film (2) of the adhesive film (3) included in the adhesive optical filter (10) is provided in contact with the encapsulation substrate (15) of the OLED panel (20).

Although not illustrated in the drawings, the OLED panel may have a dual emission structure, and when the OLED panel has a dual emission structure, the optical film may be provided on both outermost side surfaces of the OLED panel, and may also be provided on one outermost side surface of the OLED panel.

The optical film may improve visibility and display performance by minimizing external light from being reflected by a reflective layer made of a metal such as an electrode and a wire of the OLED panel and coming out of the outer side of the OLED panel. The outer side of the OLED panel means an outer side of the encapsulation substrate in the top emission, and means an outer side of the substrate in the bottom emission.

In one example, the OLED panel may further include a color filter-formed substrate as necessary. The color filter means a layer formed by coating color resists of red, green and blue in a specific pattern, and, when light passes through, displaying colors through each color filter.

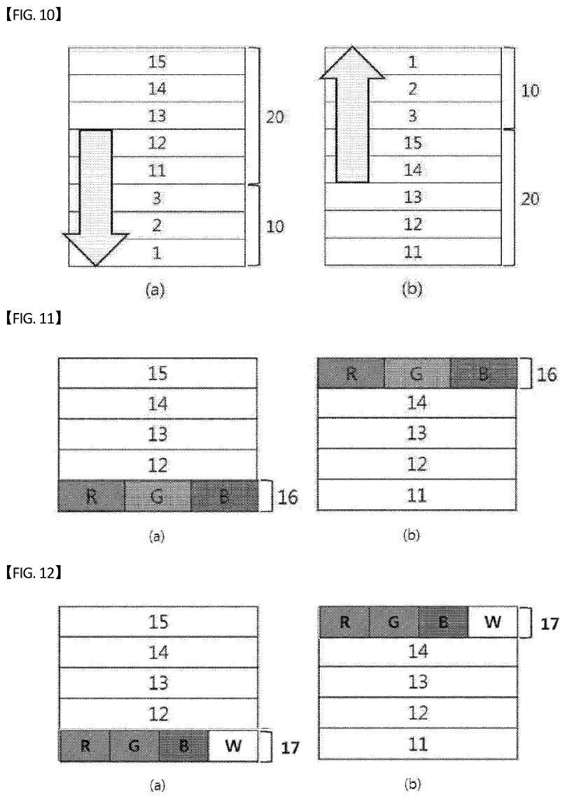

(a) of FIG. 11 illustrates a structure of the OLED panel in a bottom emission structure provided with the color filter-formed substrate (16), and the color filter-formed substrate (16) may be disposed on a surface opposite to the surface where a lower electrode (12) and an organic material layer (13) are in contact with each other. Herein, the OLED panel may have a structure consecutively including an encapsulation substrate (15), a transparent metal oxide electrode (anode) that is an upper electrode (14), the organic material layer (13), a metal electrode (cathode) that is the lower electrode (12) and the color filter-formed substrate (16).

(b) of FIG. 11 illustrates a structure of the OLED panel in a top emission structure provided with the color filter-formed substrate (16), and the color filter-formed substrate (16) may be disposed on a surface opposite to the surface where an upper electrode (14) and an organic material layer (13) are in contact with each other. Herein, the OLED panel may have a structure consecutively including the color filter-formed substrate (16), the upper electrode (14), the organic material layer (13), a lower electrode (12) and a substrate (11). As illustrated, the color filter may include red (R), green and blue regions, and although not separately indicated in the drawing, a black matrix for separating the regions may be further included. When a color filter is present in the OLED panel, lower panel reflectance may be obtained compared when a color filter is not present. Specifically, when a red, green and blue color filter is present in front of a light emitting layer of an OLED, high reflectance in a metal electrode located at the back surface of the light emitting layer is reduced. The panel reflectance means electrode reflection, and specifically means that external light penetrating into the OLED panel is reflected by an electrode included in the OLED panel.

The OLED panel may be employed without particular limit as long as it is used in the art, but may have average reflectance of approximately 30% to 50% in a wavelength range of 400 nm to 600 nm, and may also be an OLED panel with 25% or less. The average reflectance may be expressed as a sum of regular reflected light obtained by light from a light source entering the reflective surface and reflected at the same angle and diffused reflected light that is light scattered and reflected in various directions instead of being regular reflected due to irregularities or curves on the surface, and is expressed by averaging 400 nm to 600 nm reflectance values among the measured reflectance values for each wavelength.

(a) of FIG. 12 illustrates a structure of the white OLED panel in a bottom emission structure provided with a substrate having a white pixel-including color filter formed therein (17), and the substrate having a white pixel-including color filter formed therein (17) may be disposed on a surface opposite to the surface where a lower electrode (12) and an organic material layer (13) are in contact with each other. Herein, the OLED panel may have a structure consecutively including an encapsulation substrate (15), a transparent metal oxide electrode (anode) that is an upper electrode (14), the organic material layer (13), a metal electrode (cathode) that is the lower electrode (12) and the substrate having a white pixel-including color filter formed therein (17).

(b) of FIG. 12 illustrates a structure of the OLED panel in a top emission structure provided with a substrate having a white pixel-including color filter formed therein (17), and the substrate having a white pixel-including color filter formed therein (17) may be disposed on a surface opposite to the surface where an upper electrode (14) and an organic material layer (13) are in contact with each other. Herein, the OLED panel may have a structure consecutively including the substrate having a white pixel-including color filter formed therein (17), the upper electrode (14), the organic material layer (13), a lower electrode (12) and a substrate (11).

Hereinafter, the present specification will be described in detail with reference to examples. However, the examples according to the present specification may be modified to various other forms, and the scope of the present specification is not to be construed as being limited to the examples described below. The examples of the present specification are provided in order to more fully describe the present specification to those having average knowledge in the art.

### Preparation Example Compound 1

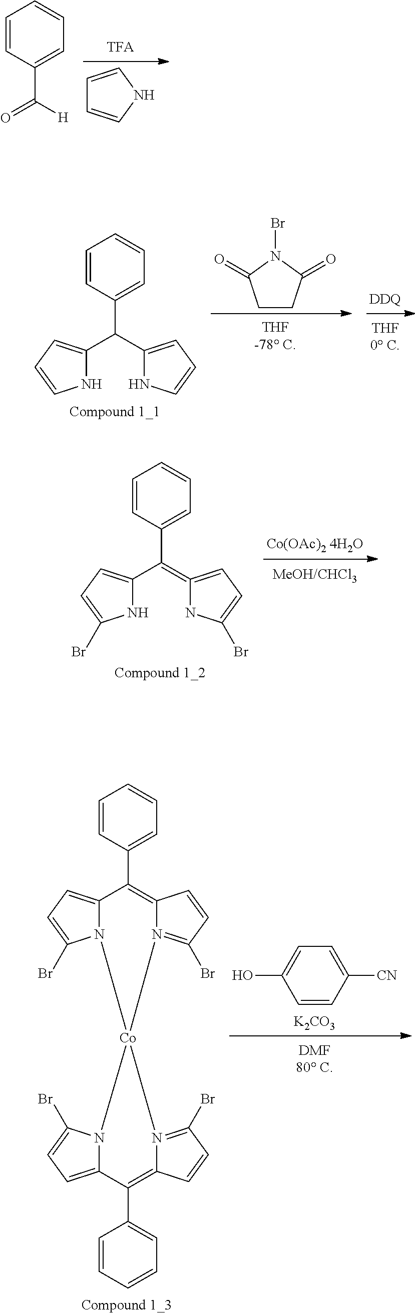

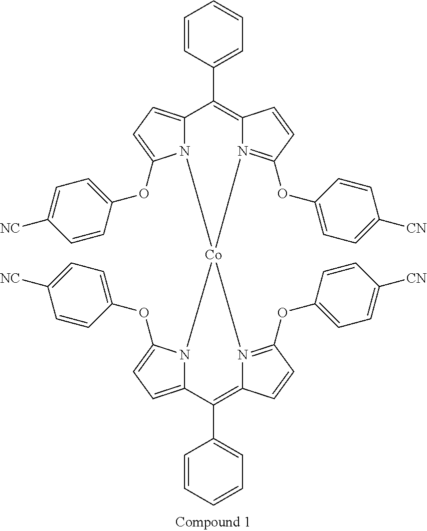

Synthesis of Compound 1_1

Benzaldehyde (20.0 g) was introduced to a pyrrole solvent and stirred well. Trifluoroacetic acid (0.10 equivalent) was slowly introduced thereto. After identifying the completion of the reaction, the result was extracted using dichloromethane and an aqueous sodium bicarbonate solution. The extracted organic layer was dried with sodium sulfate, and then a silica gel column was used to secure purified and separated Compound 1_1 (15.5 g, yield 37.0%).

Synthesis of Compound 1_2

Compound 1_1 (15.5 g) was stirred well and dissolved in a tetrahydrofuran solvent. The reaction solution was cooled to −78° C. using dry ice and acetone, and N-bromosuccinimide (NBS) (2.0 equivalent) was slowly introduced thereto in a solid state while maintaining the temperature. When the reaction was completed, the reaction solution was cooled to 0° C. using ice water, and then 2,3-dichloro-5,6-dicyano-1,4-benzoquinone (DDQ) (1.1 equivalent) was introduced thereto. When the reaction was completed, triethylamine (1.5 equivalent) was introduced thereto, and the result was extracted using diethyl ether and water. The extracted organic layer was dried with sodium sulfate, and then a silica gel column was used to secure purified and separated Compound 12 (10.2 g, yield 38.7%).

Synthesis of Compound 1_3

Compound 12 (3.0 g) was stirred well and dissolved in a methanol/chloroform (1/1) solvent. Cobalt acetate tetrahydrate (0.50 equivalent) was introduced to the reaction solution in a solid state. After the reaction was completed, the result was extracted with dichloromethane and water. The extracted organic layer was dried using sodium sulfate and, after removing the solvent by vacuum distillation, recrystallized using methanol. Through the recrystallization, purified and separated Compound 13 (2.7 g, yield 83.7%) was secured.

Synthesis of Compound 1

Compound 13 (2.7 g) was stirred well and dissolved in an N,N-dimethylformamide solvent. Sodium carbonate (20.0 equivalent) and 4-cyanophenol (10.0 equivalent) were introduced thereto, and the reaction solution was heated to 80° C. and stirred. After the reaction was completed, the result was extracted using chloroform and water. The extracted organic layer was dried with sodium sulfate and, after removing the solvent by vacuum distillation, recrystallized using methanol. Through the recrystallization, purified and separated Compound 1 (2.4 g, yield 74.8%) was secured. HR LC/MS/MS m/z calculated for C₅₈H₃₄CoN₈O₄ (M+): 965.2035; found: 965.2041.

### Preparation Example Compound 2

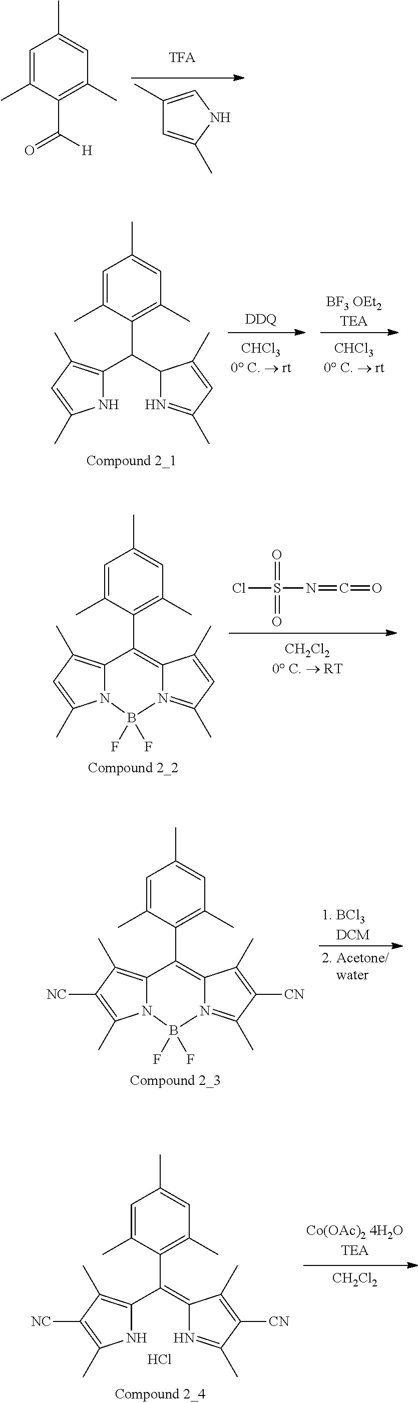

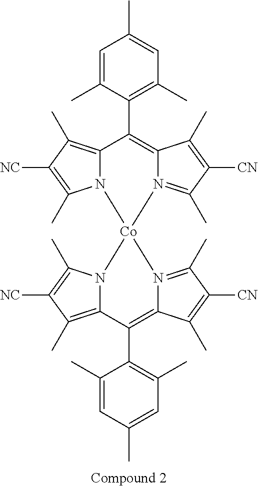

Synthesis of Compound 2_1

Mesityl aldehyde (20.0 g) was introduced to a 2,4-dimethylpyrrole solvent, and stirred well. Trifluoroacetic acid (0.10 equivalent) was slowly introduced thereto. After identifying the completion of the reaction, the result was extracted using dichloromethane and an aqueous sodium bicarbonate solution. The extracted organic layer was dried with sodium sulfate, and then a silica gel column was used to secure purified and separated Compound 2_1 (16.8 g, yield 38.8%).

Synthesis of Compound 2_2

Compound 2_1 (16.8 g) was stirred well and dissolved in a chloroform solvent. The reaction solution was cooled to 0° C. using ice water, and 2,3-dichloro-5,6-dicyano-1,4-benzoquinone (DDQ) (1.1 equivalent) was introduced thereto. The result was stirred well at room temperature, and when the reaction was completed, triethylamine (1.5 equivalent) was introduced thereto, and the result was extracted using diethyl ether and water. After drying the extracted organic layer with sodium sulfate, the reaction solution remaining in the container obtained by vacuum distilling the filtrate was stirred well again in a chloroform solvent. The reaction solution was cooled to 0° C. using ice water, and then triethylamine (20.0 equivalent) and a boron trifluoride ethyl ether complex (BF₃.OEt₂) (10.0 equivalent) were slowly introduced thereto. The reaction solution was stirred at room temperature, and when the reaction was finished, the result was extracted using chloroform and an aqueous sodium bicarbonate solution. The extracted organic layer was dried with sodium sulfate, and then a silica gel column was used to secure purified and separated Compound 2_2 (15.5 g, yield 80.7%).

Synthesis of Compound 2_3

Compound 2_2 (3.0 g) was stirred well and dissolved in a dichloromethane solvent. After cooling the reaction solution to 0° C. using ice water, chlorosulfonyl isocyanate (10.0 equivalent) was introduced thereto, and the result was stirred at room temperature. When the reaction was completed, N,N-dimethylformamide (20.0 equivalent) was introduced thereto, and the result was stirred well again for a sufficient period of time. The result was extracted using chloroform and water, and the organic layer was dried using sodium sulfate. The solvent was removed by vacuum distillation, and the result was recrystallized using methanol. Through the recrystallization, purified and separated Compound 2_3 (2.7 g, yield 79.2%) was secured.

Synthesis of Compound 2_4

Compound 2_3 (2.7 g) was stirred well and dissolved in a dichloromethane solvent. A boron trichloride 1.0 M heptane solution (1.0 equivalent) was slowly added dropwise thereto. When the reaction was completed, the solvent was vacuum distilled at a low temperature of 30° C. or lower, then acetone and water in a ratio of 10/1 were introduced to the reaction solution remaining in the container, and the result was stirred well again. When the reaction was completed, the result was extracted with dichloromethane and water, and the extracted organic layer was dried using sodium sulfate. The solvent was removed by vacuum distillation, and the result was recrystallized using methanol. Through the recrystallization, purified and separated Compound 2_4 (2.2 g, yield 83.8%) was secured.

Synthesis of Compound 2

Compound 2_4 (2.2 g) was stirred well and dissolved in a dichloromethane solvent. Cobalt acetate tetrahydrate (0.50 equivalent) was introduced to the reaction solution in a solid state, and triethylamine (2.5 equivalent) was further introduced thereto. After the reaction was completed, the result was extracted with dichloromethane and water. The extracted organic layer was dried using sodium sulfate and, after removing the solvent by vacuum distillation, recrystallized using methanol. Through the recrystallization, purified and separated Compound 2 (1.9 g, yield 88.1%) was secured. HR LC/MS/MS m/z calculated for C₄₈H₄₆CoN₈ (M+): 793.3177; found: 793.3183.

### Preparation Example Compound 3

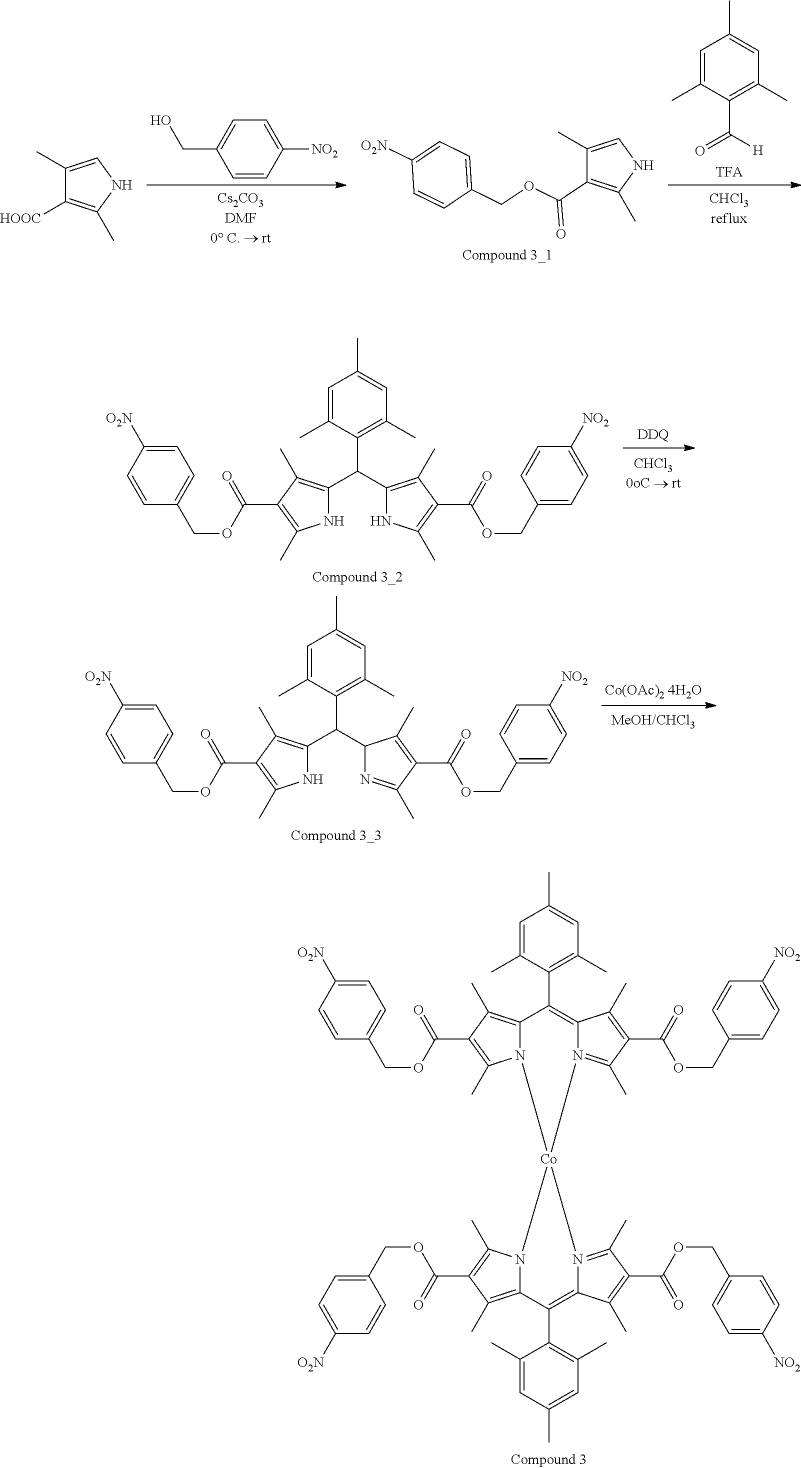

Synthesis of Compound 3_1

2,4-Dimethylpyrrole-3-carboxylic acid (10.0 g) was stirred well and dissolved in an N,N-dimethylformamide solvent. The reaction solution was cooled to 0° C. using ice water, and cesium carbonate (3.0 equivalent) and 4-nitrobenzyl alcohol (2.0 equivalent) were introduced thereto. After that, the reaction solution was stirred at room temperature. After the reaction was completed, the result was extracted using chloroform and water. The extracted organic layer was dried with sodium sulfate and, after removing the solvent by vacuum distillation, recrystallized using methanol. Through the recrystallization, purified and separated Compound 3_1(5.8 g, yield 29.4%) was secured.

Synthesis of Compound 3_2

Mesityl aldehyde (2.0 g) was introduced to a chloroform solvent and stirred well. Compound 3_1 (2.0 equivalent) was introduced thereto, and then trifluoroacetic acid (0.20 equivalent) was slowly introduced thereto. The reaction solution was stirred under reflux, and completion of the reaction was identified. The reaction solution was extracted using chloroform and an aqueous sodium bicarbonate solution. The extracted organic layer was dried with sodium sulfate, and then a silica gel column was used to secure purified and separated Compound 3_2 (3.8 g, yield 41.5%).

Synthesis of Compound 3_3

Compound 3_2 (3.8 g) was stirred well and dissolved in a chloroform solvent. The reaction solution was cooled to 0° C. using ice water, and 2,3-dichloro-5,6-dicyano-1,4-benzoquinone (DDQ) (1.1 equivalent) was introduced thereto. The result was stirred well at room temperature, and when the reaction was completed, triethylamine (1.5 equivalent) was introduced thereto, and the result was extracted using diethyl ether and water. The extracted organic layer was dried with sodium sulfate, and then a silica gel column was used to secure purified and separated Compound 33 (3.1 g, yield 81.8%).

Synthesis of Compound 3

Compound 3_3 (3.1 g) was stirred well and dissolved in a methanol/chloroform (1/1) solvent. Cobalt acetate tetrahydrate (0.50 equivalent) was introduced to the reaction solution in a solid state. After the reaction was completed, the result was extracted with dichloromethane and water. The extracted organic layer was dried using sodium sulfate and, after removing the solvent by vacuum distillation, recrystallized using methanol. Through the recrystallization, purified and separated Compound 3 (2.8 g, yield 86.7%) was secured. HR LC/MS/MS m/z calculated for C₇₆H₇₀CoN₈O₁₆ (M+): 1409.4242; found: 1409.4248.

### Preparation Example Compound 4

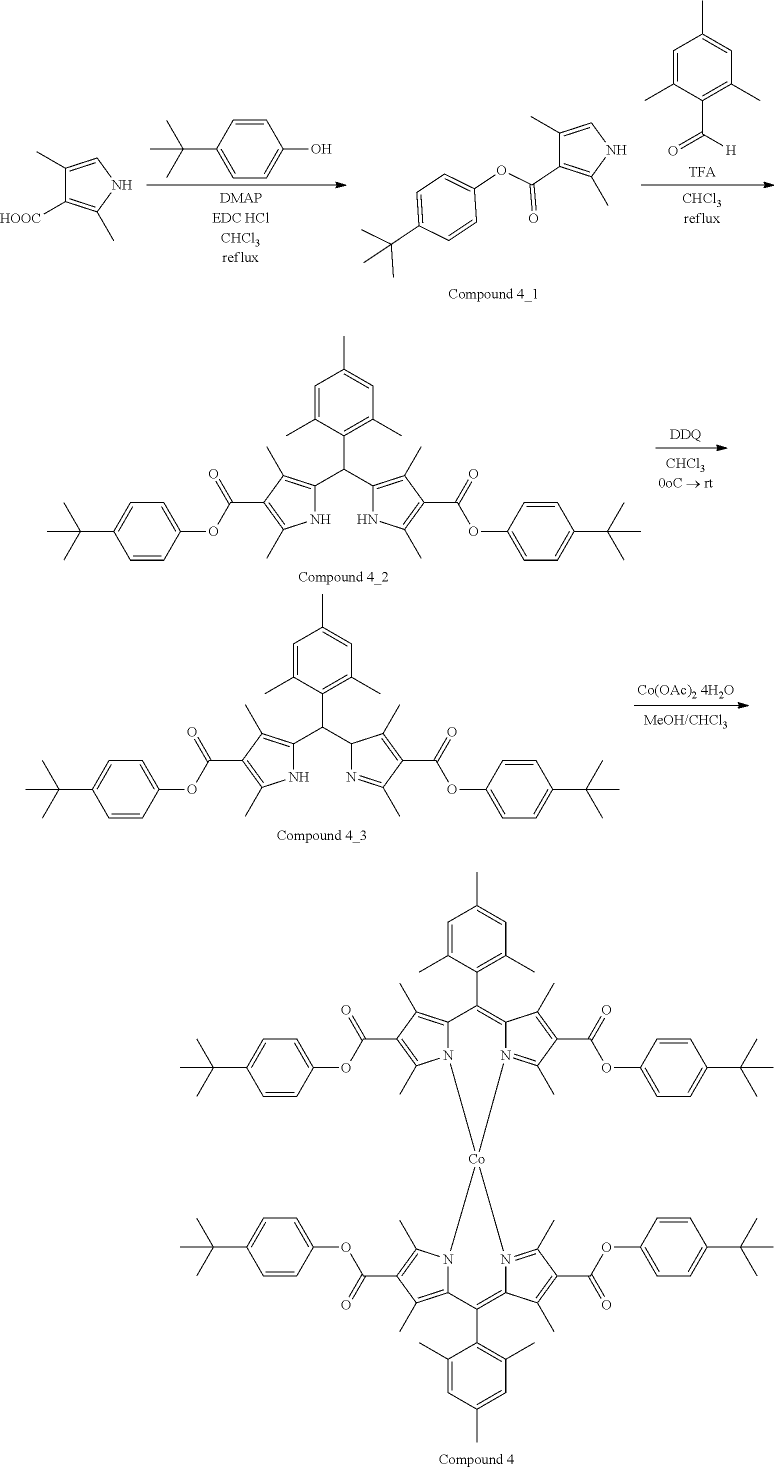

Synthesis of Compound 4_1

2,4-Dimethylpyrrole-3-carboxylic acid (10.0 g) was stirred well and dissolved in a chloroform solvent. 4-t-Butylphenol (2.0 equivalent), 4-dimethylaminopyridine (DMAP) (2.2 equivalent) and N-(3-dimethylaminopropyl)-N′-ethylcarbodiimide hydrochloride (EDC-HCl) (2.2 equivalent) were introduced thereto, and the result was stirred under reflux. After the reaction was completed, the reaction solution was cooled to room temperature, and extracted using chloroform and water. The extracted organic layer was dried with sodium sulfate and, after removing the solvent by vacuum distillation, recrystallized using ethanol. Through the recrystallization, purified and separated Compound 4_1 (9.5 g, yield 48.7%) was secured.

Synthesis of Compound 4_2

Mesityl aldehyde (3.0 g) was introduced to a chloroform solvent and stirred well. Compound 4_1 (2.0 equivalent) was introduced thereto, and then trifluoroacetic acid (0.20 equivalent) was slowly introduced thereto. The reaction solution was stirred under reflux, and completion of the reaction was identified. The reaction solution was extracted using chloroform and an aqueous sodium bicarbonate solution. The extracted organic layer was dried with sodium sulfate, and then a silica gel column was used to secure purified and separated Compound 42 (5.9 g, yield 43.3%).

Synthesis of Compound 4_3

Compound 4_2 (5.9 g) was stirred well and dissolved in a chloroform solvent. The reaction solution was cooled to 0° C. using ice water, and 2,3-dichloro-5,6-dicyano-1,4-benzoquinone (DDQ) (1.1 equivalent) was introduced thereto. The result was stirred well at room temperature, and when the reaction was completed, triethylamine (1.5 equivalent) was introduced thereto, and the result was extracted using diethyl ether and water. The extracted organic layer was dried with sodium sulfate, and then a silica gel column was used to secure purified and separated Compound 43 (5.1 g, yield 86.7%).

Synthesis of Compound 4

Compound 4_3 (3.0 g) was stirred well and dissolved in a methanol/chloroform (1/1) solvent. Cobalt acetate tetrahydrate (0.50 equivalent) was introduced to the reaction solution in a solid state. After the reaction was completed, the result was extracted with dichloromethane and water. The extracted organic layer was dried using sodium sulfate and, after removing the solvent by vacuum distillation, recrystallized using methanol. Through the recrystallization, purified and separated Compound 4 (2.7 g, yield 86.3%) was secured. HR LC/MS/MS m/z calculated for C₈₈H₉₈CoN₄O₈ (M+): 1397.6717; found: 1397.6723.

### Preparation Example Compound 5

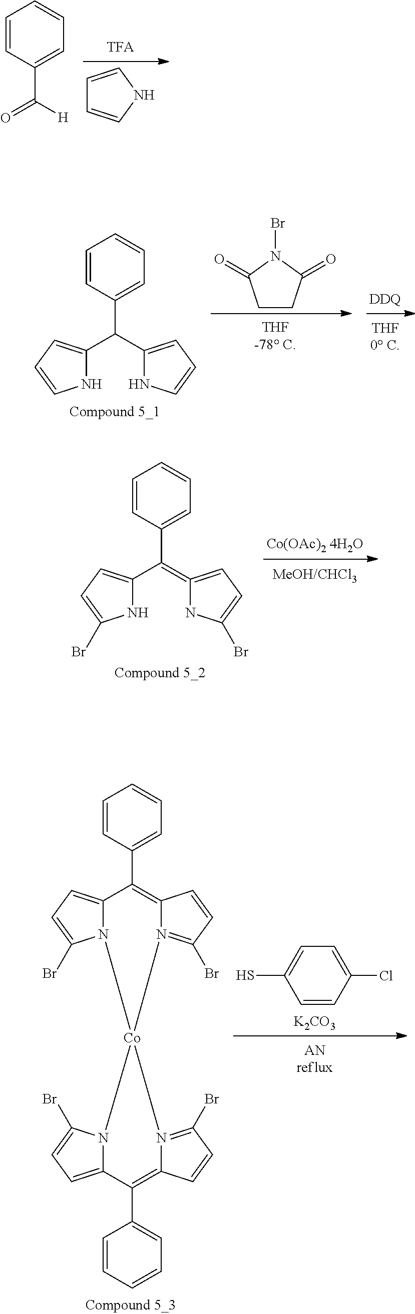

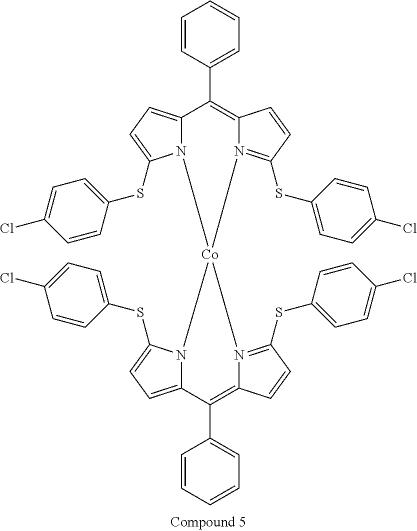

Synthesis of Compound 5_1

Benzaldehyde (20.0 g) was introduced to a pyrrole solvent and stirred well. Trifluoroacetic acid (0.10 equivalent) was slowly introduced thereto. After identifying the completion of the reaction, the result was extracted using dichloromethane and an aqueous sodium bicarbonate solution. The extracted organic layer was dried with sodium sulfate, and then a silica gel column was used to secure purified and separated Compound 5_1 (8.5 g, yield 20.3%).

Synthesis of Compound 5_2

Compound 5_1 (8.5 g) was stirred well and dissolved in a tetrahydrofuran solvent. The reaction solution was cooled to −78° C. using dry ice and acetone, and N-bromosuccinimide (NBS) (2.0 equivalent) was slowly introduced thereto in a solid state while maintaining the temperature. When the reaction was completed, the reaction solution was cooled to 0° C. using ice water, and then 2,3-dichloro-5,6-dicyano-1,4-benzoquinone (DDQ) (1.1 equivalent) was introduced thereto. When the reaction was completed, triethylamine (1.5 equivalent) was introduced thereto, and the result was extracted using diethyl ether and water. The extracted organic layer was dried with sodium sulfate, and then a silica gel column was used to secure purified and separated Compound 52 (7.7 g, yield 53.3%).

Synthesis of Compound 5_3

Compound 5_2 (3.0 g) was stirred well and dissolved in a methanol/chloroform (1/1) solvent. Cobalt acetate tetrahydrate (0.50 equivalent) was introduced to the reaction solution in a solid state. After the reaction was completed, the result was extracted with dichloromethane and water. The extracted organic layer was dried using sodium sulfate and, after removing the solvent by vacuum distillation, recrystallized using methanol. Through the recrystallization, purified and separated Compound 5_3 (2.6 g, yield 80.6%) was secured.

Synthesis of Compound 5

Compound 5_3 (2.6 g) was stirred well and dissolved in an acetonitrile solvent. Sodium carbonate (6.0 equivalent) and 4-chlorothiophenol (6.0 equivalent) were introduced thereto, and the reaction solution was heated to 80° C. and stirred under reflux. After the reaction was completed, the result was extracted using chloroform and water. The extracted organic layer was dried with sodium sulfate and, after removing the solvent by vacuum distillation, recrystallized using methanol. Through the recrystallization, purified and separated Compound 5 (2.8 g, yield 82.0%) was secured. HR LC/MS/MS m/z calculated for C₅₄H₃₄Cl₄CoN₄S₄ (M+): 1064.9752; found: 1064.9759.

### Preparation Example Compound 6

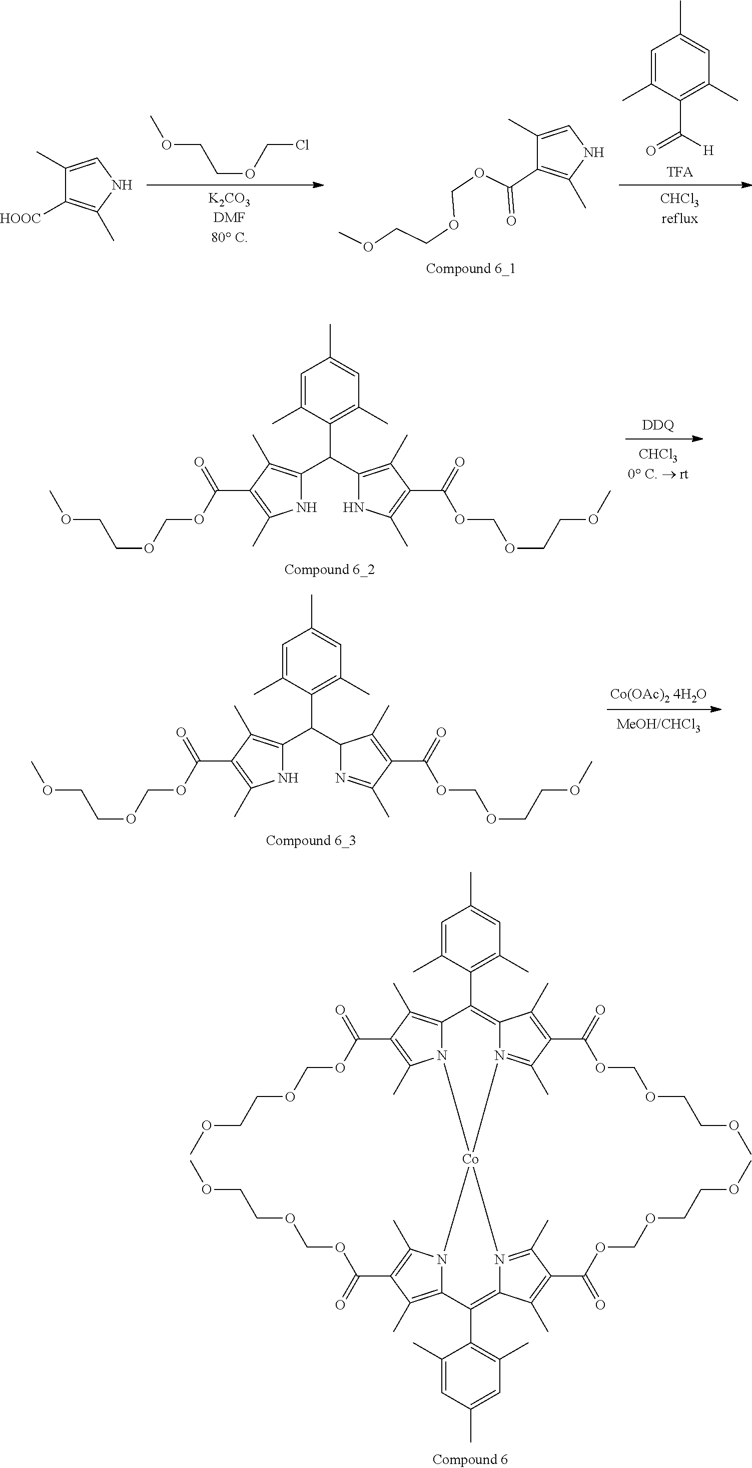

Synthesis of Compound 6_1

2,4-Dimethylpyrrole-3-carboxylic acid (10.0 g) was stirred well and dissolved in an N,N-dimethylformamide solvent. Sodium carbonate (3.0 equivalent) and 2-methoxyethoxymethyl chloride (2.0 equivalent) were introduced thereto, and the reaction solution was heated to 80° C. and stirred. After the reaction was completed, the result was extracted using chloroform and water. The extracted organic layer was dried with sodium sulfate and, after removing the solvent by vacuum distillation, recrystallized using methanol. Through the recrystallization, purified and separated Compound 6_1 (7.8 g, yield 47.8%) was secured.

Synthesis of Compound 6_2

Mesityl aldehyde (1.5 g) was introduced to a chloroform solvent and stirred well. Compound 6_1 (2.0 equivalent) was introduced thereto, and then trifluoroacetic acid (0.20 equivalent) was slowly introduced thereto. The reaction solution was stirred under reflux, and completion of the reaction was identified. The reaction solution was extracted using chloroform and an aqueous sodium bicarbonate solution. The extracted organic layer was dried with sodium sulfate, and then a silica gel column was used to secure purified and separated Compound 6_2 (2.9 g, yield 49.0%).

Synthesis of Compound 6_3

Compound 6_2 (2.9 g) was stirred well and dissolved in a chloroform solvent. The reaction solution was cooled to 0° C. using ice water, and 2,3-dichloro-5,6-dicyano-1,4-benzoquinone (DDQ) (1.1 equivalent) was introduced thereto. The result was stirred well at room temperature, and when the reaction was completed, triethylamine (1.5 equivalent) was introduced thereto, and the result was extracted using diethyl ether and water. The extracted organic layer was dried with sodium sulfate, and then a silica gel column was used to secure purified and separated Compound 6_3 (2.2 g, yield 76.1%).

Synthesis of Compound 6

Compound 6_3 (2.2 g) was stirred well and dissolved in a methanol/chloroform (1/1) solvent. Cobalt acetate tetrahydrate (0.50 equivalent) was introduced to the reaction solution in a solid state. After the reaction was completed, the result was extracted with dichloromethane and water. The extracted organic layer was dried using sodium sulfate and, after removing the solvent by vacuum distillation, recrystallized using methanol. Through the recrystallization, purified and separated Compound 6 (2.0 g, yield 86.7%) was secured. HR LC/MS/MS m/z calculated for C₆₄H₈₂CoN₄O₁₆ (M+): 1221.5058; found: 1221.5063.

### Preparation Example Compound 7

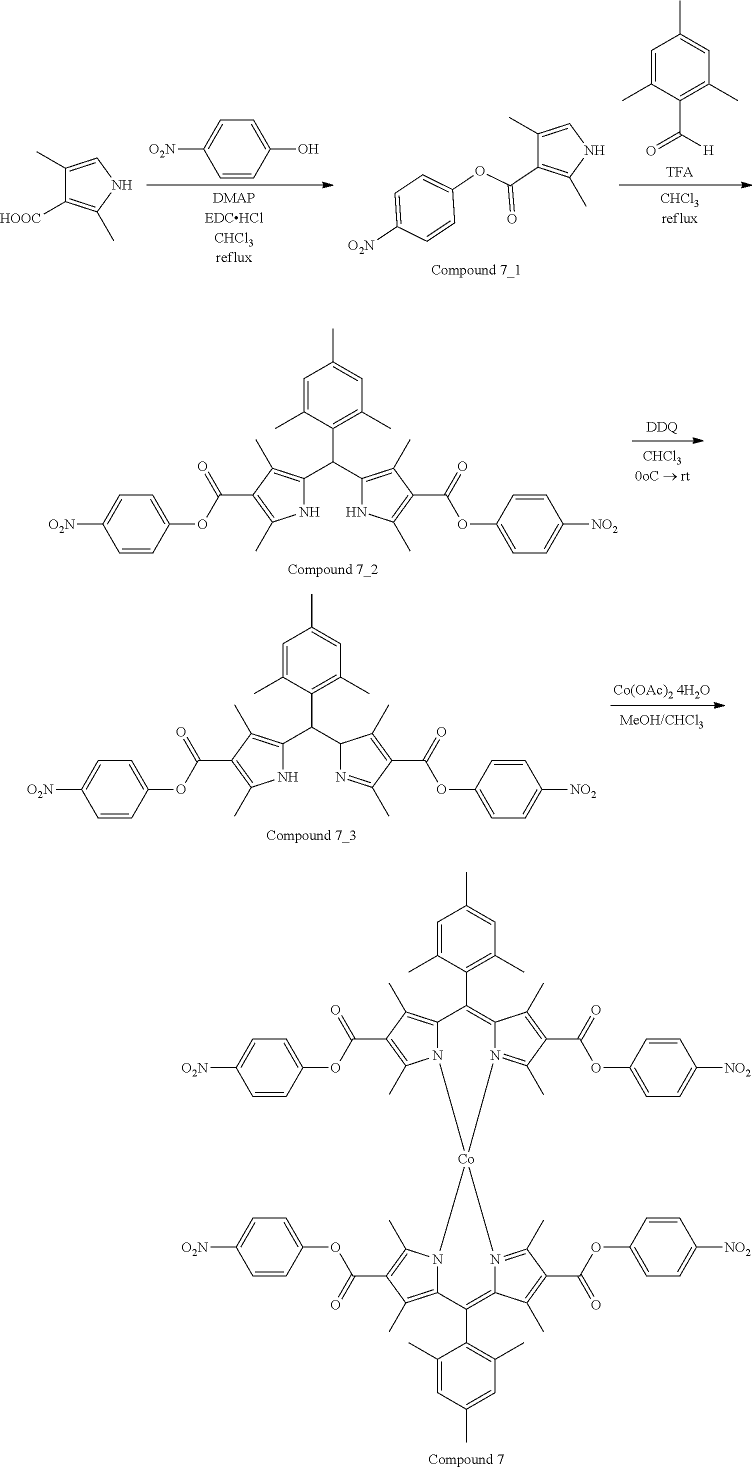

Synthesis of Compound 7_1

2,4-Dimethylpyrrole-3-carboxylic acid (10.0 g) was introduced to a chloroform solvent, stirred well and dissolved therein. 4-Nitrophenol (2.0 equivalent), 4-dimethylaminopyridine (DMAP) (2.2 equivalent) and N-(3-dimethylaminopropyl)-N′-ethylcarbodiimide hydrochloride (EDC-HCl) (2.2 equivalent) were introduced thereto, and the result was stirred under reflux. After the reaction was completed, the reaction solution was cooled to room temperature, and extracted using chloroform and water. The extracted organic layer was dried with sodium sulfate and, after removing the solvent by vacuum distillation, recrystallized using ethanol. Through the recrystallization, purified and separated Compound 7_1(10.9 g, yield 58.3%) was secured.

Synthesis of Compound 7_2

Mesityl aldehyde (2.0 g) was introduced to a chloroform solvent and stirred well. Compound 7_1 (2.0 equivalent) was introduced thereto, and then trifluoroacetic acid (0.20 equivalent) was slowly introduced thereto. The reaction solution was stirred under reflux, and completion of the reaction was identified. The reaction solution was extracted using chloroform and an aqueous sodium bicarbonate solution. The extracted organic layer was dried with sodium sulfate, and then a silica gel column was used to secure purified and separated Compound 72 (3.7 g, yield 42.1%).

Synthesis of Compound 7_3

Compound 7_2 (3.0 g) was stirred well and dissolved in a chloroform solvent. The reaction solution was cooled to 0° C. using ice water, and 2,3-dichloro-5,6-dicyano-1,4-benzoquinone (DDQ) (1.1 equivalent) was introduced thereto. The result was stirred well at room temperature, and when the reaction was completed, triethylamine (1.5 equivalent) was introduced thereto, and the result was extracted using diethyl ether and water. The extracted organic layer was dried with sodium sulfate, and then a silica gel column was used to secure purified and separated Compound 7_3 (2.5 g, yield 83.6%).

Synthesis of Compound 7

Compound 7_3 (2.5 g) was stirred well and dissolved in a methanol/chloroform (1/1) solvent. Cobalt acetate tetrahydrate (0.50 equivalent) was introduced to the reaction solution in a solid state. After the reaction was completed, the result was extracted with dichloromethane and water. The extracted organic layer was dried using sodium sulfate and, after removing the solvent by vacuum distillation, recrystallized using methanol. Through the recrystallization, purified and separated Compound 7 (2.2 g, yield 84.3%) was secured. HR LC/MS/MS m/z calculated for C₇₂H₆₂CoN₈O₁₆ (M+): 1353.3616; found: 1353.3624.

### Preparation Example Compound 8

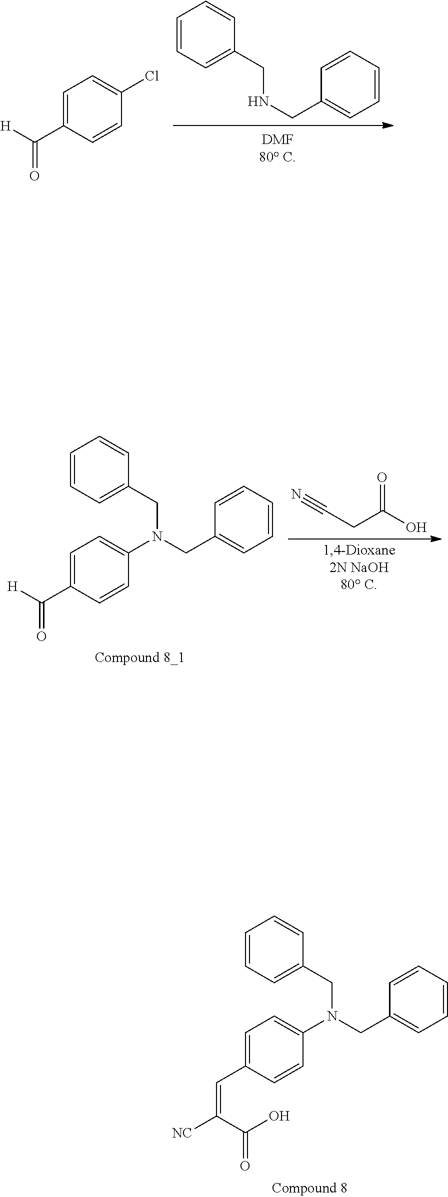

Preparation of Compound 8_1

4-Chlorobenzaldehyde (20.0 g) was stirred well in an N,N-dimethylformamide solvent. Dibenzylamine (1.1 equivalent) was introduced to the reaction solution, and the result was heated to 80° C. and stirred. After the reaction was completed, the result was cooled to room temperature, diluted with ethyl acetate, and washed 3 times with water. The collected organic layer was dried with anhydrous magnesium sulfate, and the solvent was removed by vacuum distillation. A silica gel column was used to secure purified and separated Compound 8_1 (26.1 g, yield 60.9%).

Preparation of Compound 8

Compound 8_1 (15.0 g) was stirred well in a 1,4-dioxane solvent. 2-Cyanoacetic acid (1.1 equivalent) was introduced to the reaction solution, an aqueous 2 N NaOH solution (10.0 mL) was added thereto, and the result was heated to 80° C. and stirred. After the reaction was finished, the result was cooled to room temperature, and 2 N HCl was added thereto to adjust the pH to neutral, and then precipitated solids were filtered. The filtered solids were slurried with hexane and isopropyl alcohol solvents to secure Compound 8 (17.2 g, yield 93.8%). HR LC/MS/MS m/z calculated for C₂₄H₂₀N₂O₂ (M+): 368.436; found: 368.442.

### Experimental Example

### Example 1

A composition for forming an optical film was prepared by adding, with respect to 100 parts by weight (solid content 15.5 parts by weight) of a butyl acrylate (BA)/hydroxyethyl acrylate (HEA) copolymer solution as an acrylate-based resin, 0.017 parts by weight of Compound 1, 0.016 parts by weight of an isocyanate-based crosslinking agent (T39M, Soken Chemical & Engineering Co., Ltd.), 0.035 parts by weight of a silane-based coupling agent (T-789J, Soken Chemical & Engineering Co., Ltd.), 0.002 parts by weight of a catalyst (dibutyltin dilaurate, Sigma-Aldrich), 0.8 parts by weight of an antioxidant (Kinox-80, Hannong Chemicals, Inc.), 0.14 parts by weight of an antistatic agent (FC-4400, 3M Corporation) and 0.031 parts by weight of a hindered amine light stabilizer (Tinuvin 123, BASF Corporation), and adding a solvent (MEK) in 25% by weight with respect to the total weight of the composition for forming an optical film.

The composition for forming an optical film mixed using a shaker (SKC 6100, JEIO Tech.) was coated on a release layer (PET) to a thickness of 22 μm to 23 μm using a knife bar coating device (KP-3000, Kipae E&T) to prepare an adhesive film. After the coating, the release layer was removed, and the adhesive film and a binder resin film (TAC: cellulose triacetate) were consecutively laminated on glass through lamination to prepare a sample.

Immediately after preparing the sample, transmittance of the sample was measured according to the CIE 1976 color coordinate using a UV-vis apparatus (Shimazu UV-3600). After exposing the sample to each of a heat resistance 80° C. 500 h condition, a moisture resistance 60° C. 90% RH 500 h condition and a light resistance 22,500,000 lx*hr condition, transmittance was remeasured. The value obtained by subtracting the transmittance immediately after preparing the sample from the remeasured transmittance was divided by the transmittance immediately after preparing the sample to calculate ΔY as a percentage, and the results are described in Table 2 by the following evaluation condition.

[Evaluation Condition]

∘: ΔY of 1.5% or less

Δ: ΔY of greater than 1.5% and 3% or less

X: ΔY of greater than 3%

### Example 2

A sample was prepared and measured in the same manner as in Example 1 except that 0.008 parts by weight of Compound 2 was used instead of Compound 1.

### Example 3

A sample was prepared and measured in the same manner as in Example 1 except that 0.014 parts by weight of Compound 3 was used instead of Compound 1.

### Example 4

A sample was prepared and measured in the same manner as in Example 1 except that 0.014 parts by weight of Compound 4 was used instead of Compound 1.

### Example 5

A sample was prepared and measured in the same manner as in Example 1 except that 0.022 parts by weight of Compound 5 was used instead of Compound 1.

### Example 6

A sample was prepared and measured in the same manner as in Example 1 except that 0.017 parts by weight of Compound 1 and 0.019 parts by weight of Compound 8 were used instead of Compound 1.

### Example 7

A sample was prepared and measured in the same manner as in Example 1 except that 0.008 parts by weight of Compound 2 and 0.026 parts by weight of Solvent Yellow 93 were used instead of Compound 1.

### Example 8

A sample was prepared and measured in the same manner as in Example 1 except that 0.014 parts by weight of Compound 3 and 0.078 parts by weight of Solvent Black 29 were used instead of Compound 1.

### Example 9

A sample was prepared and measured in the same manner as in Example 1 except that 0.014 parts by weight of Compound 4 and 0.054 parts by weight of Blue S-3R were used instead of Compound 1.

### Example 10

A sample was prepared and measured in the same manner as in Example 1 except that 0.020 parts by weight of Compound 5 and 0.012 parts by weight of FDG-007 were used instead of Compound 1.

### Example 11

A sample was prepared and measured in the same manner as in Example 1 except that 0.11 parts by weight of Compound 1, 0.09 parts by weight of Solvent Yellow 179 and 0.07 parts by weight of C₅₈₃CL were used instead of Compound 1.

### Example 12

A sample was prepared and measured in the same manner as in Example 1 except that 0.020 parts by weight of Compound 5, 0.088 parts by weight of Solvent Yellow 163 and 0.011 parts by weight of FDG-007 were used instead of Compound 1.

### Example 13

A sample was prepared and measured in the same manner as in Example 1 except that 0.014 parts by weight of Compound 4, 0.011 parts by weight of C₅₉₂VL and 0.053 parts by weight of Solvent Blue 67 were used instead of Compound 1.

### Example 14

A sample was prepared and measured in the same manner as in Example 1 except that 0.011 parts by weight of Compound 6 was used instead of Compound 1.

### Example 15

A sample was prepared and measured in the same manner as in Example 1 except that 0.009 parts by weight of Compound 7 was used instead of Compound 1.

### Example 16

A sample was prepared and measured in the same manner as in Example 1 except that 0.011 parts by weight of Compound 6 and 0.034 parts by weight of Solvent Black 34 were used instead of Compound 1.

### Example 17

A sample was prepared and measured in the same manner as in Example 1 except that 0.009 parts by weight of Compound 7 and 0.011 parts by weight of C592VL were used instead of Compound 1.

### Comparative Example 1

A sample was prepared and measured in the same manner as in Example 1 except that 0.008 parts by weight of FDB-007 (Yamada Chem.) was used instead of Compound 1.

### Comparative Example 2

A sample was prepared and measured in the same manner as in Example 1 except that 0.016 parts by weight of SD-021SP was used instead of Compound 1.

### Comparative Example 3

A sample was prepared and measured in the same manner as in Example 1 except that 0.008 parts by weight of FDB-007 (Yamada Chem.) and 0.019 parts by weight of Compound 8 were used instead of Compound 1.

### Comparative Example 4

A sample was prepared and measured in the same manner as in Example 1 except that 0.011 parts by weight of SD-021SP and 0.122 parts by weight of Solvent Black 29 were used instead of Compound 1.

### Comparative Example 5

A sample was prepared and measured in the same manner as in Example 1 except that 0.008 parts by weight of FDB-007 (Yamada Chem.), 0.012 parts by weight of Solvent Yellow 179 and 0.011 parts by weight of C583CL were used instead of Compound 1.

### Comparative Example 6

A sample was prepared and measured in the same manner as in Example 1 except that 0.014 parts by weight of SD-021SP, 0.009 parts by weight of C592VL and 0.071 parts by weight of Solvent Blue 67 were used instead of Compound 1.

Each of the dyes or pigments used in Examples 1 to 17 and Comparative Examples 1 to 6 are summarized in the following Table 1.

TABLE 1

| | Dye/Pigment 1 | Dye/Pigment 2 | Dye/Pigment 3 |
|---|---|---|---|
| Example 1 | Compound 1 | | |
| Example 2 | Compound 2 | | |
| Example 3 | Compound 3 | | |
| Example 4 | Compound 4 | | |
| Example 5 | Compound 5 | | |
| Example 6 | Compound 1 | Compound 8 | |
| Example 7 | Compound 2 | Solvent | |
| | | Yellow 93 | |
| | | (Lanxess) | |
| Example 8 | Compound 3 | Solvent | |
| | | Black 29 | |
| | | (BASF) | |
| Example 9 | Compound 4 | Blue S-3R | |
| | | (Yedam Chem.) | |
| Example 10 | Compound 5 | FDG-007 | |
| | | (Yamada | |
| | | Chem.) | |
| Example 11 | Compound 1 | Solvent | C583CL |
| | | Yellow 179 | (Kyungin |
| | | (Lanxess) | Chem.) |
| Example 12 | Compound 5 | Solvent | FDG-007 |
| | | Yellow 163 | (Yamada |
| | | (Yedam Chem.) | Chem.) |
| Example 13 | Compound 4 | C592VL | Solvent |
| | | (Kyungin | Blue 67 |
| | | Chem.) | (BASF) |
| Example 14 | Compound 6 | | |
| Example 15 | Compound 7 | | |
| Example 16 | Compound 6 | Solvent | |
| | | Black 34 | |
| | | (Orient) | |
| Example 17 | Compound 7 | C592VL | |
| | | (Kyungin | |
| | | Chem.) | |
| Comparative | FDB-007 | | |
| Example 1 | (Yamada | | |
| | Chem.) | | |
| Comparative | SD-021SP | | |
| Example 2 | (Mitsui | | |
| | Chem.) | | |
| Comparative | FDB-007 | Compound 8 | |
| Example 3 | (Yamada | | |
| | Chem.) | | |
| Comparative | SD-021SP | Solvent | |
| Example 4 | (Mitsui | Black 29 | |
| | Chem.) | (BASF) | |
| Comparative | FDB-007 | Solvent | C583CL |
| Example 5 | (Yamada | Yellow 179 | (Kyungin |
| | Chem.) | (Lanxess) | Chem.) |
| Comparative | SD-021SP | C592VL | Solvent |
| Example 6 | (Mitsui | (Kyungin | Blue 67 |
| | Chem.) | Chem.) | (BASF) |

TABLE 2

| | | Moisture | |
|---|---|---|---|
| | Heat | Resistance | Light |
| | Resistance | 60° C. 90% | Resistance |
| | 80° C. 500 h | RH 500 h | 22,500,000 lx*hr |
| | Condition | Condition | Condition |
| Example 1 | ◯ | ◯ | Δ |
| Example 2 | ◯ | ◯ | ◯ |
| Example 3 | ◯ | ◯ | ◯ |
| Example 4 | ◯ | ◯ | ◯ |
| Example 5 | ◯ | ◯ | ◯ |
| Example 6 | ◯ | ◯ | Δ |
| Example 7 | ◯ | ◯ | ◯ |
| Example 8 | ◯ | ◯ | ◯ |
| Example 9 | ◯ | ◯ | ◯ |
| Example 10 | Δ | ◯ | Δ |
| Example 11 | Δ | ◯ | Δ |
| Example 12 | Δ | ◯ | Δ |
| Example 13 | ◯ | ◯ | ◯ |
| Example 14 | Δ | Δ | ◯ |
| Example 15 | ◯ | ◯ | ◯ |
| Example 16 | Δ | Δ | ◯ |
| Example 17 | ◯ | ◯ | ◯ |
| Comparative | ◯ | ◯ | X |
| Example 1 | | | |
| Comparative | ◯ | ◯ | X |
| Example 2 | | | |
| Comparative | ◯ | ◯ | X |
| Example 3 | | | |
| Comparative | ◯ | ◯ | X |
| Example 4 | | | |
| Comparative | Δ | ◯ | X |
| Example 5 | | | |
| Comparative | ◯ | ◯ | X |
| Example 6 | | | |

According to Table 2, it was identified that the examples according to the present specification were all superior in the properties of heat resistance, moisture resistance and light resistance compared to the comparative examples. Particularly, Examples 1 to 17 had very superior light resistance properties compared to Comparative Examples 1 to 6. It was identified that this was the effect obtained by superior reliability of the compound represented by Chemical Formula 1 when using the optical film including the composition for forming an optical film according to the present specification in a display device such as an OLED device.

1. A composition for forming an optical film, comprising:
a compound represented by the following Chemical Formula 1; and
a binder resin: 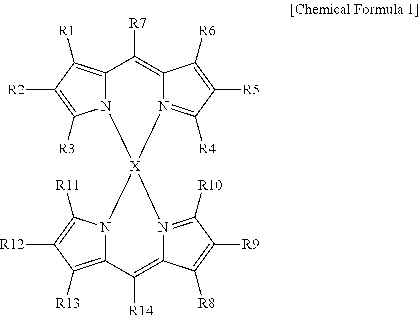
wherein, in the Chemical Formula 1,
X is Zn; Co; Ni or Pd;
R1 to R6 and R8 to R13 are the same as or different from each other, and each independently hydrogen; deuterium; a halogen group; a nitrile group; a nitro group; —OC(═O)R; —(C═O)NR′R″; —(C═O)OR′″; —(C═O)Rx; a substituted or unsubstituted alkyl group; a substituted or unsubstituted cycloalkyl group; a substituted or unsubstituted aryl group; a substituted or unsubstituted alkoxy group; a substituted or unsubstituted aryloxy group; a substituted or unsubstituted heteroaryloxy group; a substituted or unsubstituted alkylthio group; a substituted or unsubstituted arylthio group; a substituted or unsubstituted heteroarylthio group; or a substituted or unsubstituted heteroaryl group;
R7 and R14 are the same as or different from each other, and each independently hydrogen; a substituted or unsubstituted alkyl group; a substituted or unsubstituted aryl group; a substituted or unsubstituted heteroaryl group; a substituted or unsubstituted aryloxy group; a substituted or unsubstituted arylthio group; or a substituted or unsubstituted heteroaryloxy group;
when R7 and R14 are hydrogen; a substituted or unsubstituted alkyl group; a substituted or unsubstituted aryl group; or a substituted or unsubstituted heteroaryl group and R2, R5, R9 and R12 are —C(═O)OR′″, R′″ is a substituted or unsubstituted aryl group; a substituted or unsubstituted cycloalkyl group; a substituted alkyl group; or a branched unsubstituted alkyl group having 3 to 30 carbon atoms; and
R, R′, R″, R′″ and Rx are the same as or different from each other, and each independently hydrogen; a substituted or unsubstituted alkyl group: a substituted or unsubstituted cycloalkyl group: a substituted or unsubstituted heterocyclic group; or a substituted or unsubstituted aryl group, or R′ and R″ bind to each other to form a ring.

2. The composition for forming an optical film of claim 1, further comprising at least one selected from the group consisting of an antioxidant, an antistatic agent, a hindered amine-based light stabilizer, a UV stabilizer, a crosslinking agent, a coupling agent, a catalyst and a solvent.

3. The composition for forming an optical film of claim 1, wherein the binder resin includes a styrene-based resin or elastomer, an urethane-based resin or elastomer, a polyolefin-based resin or elastomer, a polyoxyalkylene-based resin or elastomer, a polyester-based resin or elastomer, a polyvinyl chloride-based resin or elastomer, a polycarbonate-based resin or elastomer, a polyphenylene sulfide-based resin or elastomer, a polyamide-based resin or elastomer, an acrylate-based resin or elastomer, an epoxy-based resin or elastomer, a silicone-based resin or elastomer, a fluorine-based resin or elastomer, or a mixture thereof.

4. The composition for forming an optical film of claim 1, further comprising an azo-based dye or pigment, a metal-containing azo-based dye or pigment, a quinoline-based dye or pigment, a methine-based dye or pigment, a coumarin-based dye or pigment, a porphyrin-based dye or pigment, an azaporphyrin-based dye or pigment, a phthalocyanine-based dye or pigment, an anthraquinone-based dye or pigment, a perylene-based dye or pigment, a squarylium-based dye or pigment, a benzoazole-based dye or pigment, or a triazine-based dye or pigment.

5. An optical film comprising the composition for forming an optical film of claim 1 or a cured product thereof.

6. An adhesive optical filter comprising:
the optical film of claim 5; and
a surface treatment layer.

7. A display device comprising the optical film of claim 5.

8. The display device of claim 7, comprising a display panel, and the optical film is provided on the display panel.

9. The display device of claim 8, wherein the display panel is a spontaneous light emitting display panel or a non-spontaneous light emitting display panel.

10. The display device of claim 8, wherein the display panel is an OLED panel; and
the display device is an OLED device comprising the OLED panel; and the optical film provided on one surface of the OLED panel.

11. The display device of claim 10, wherein the OLED panel further comprises a white pixel.

12. The composition for forming an optical film of claim 1, wherein the Chemical Formula 1 is represented by the following Chemical Formula 1-1: 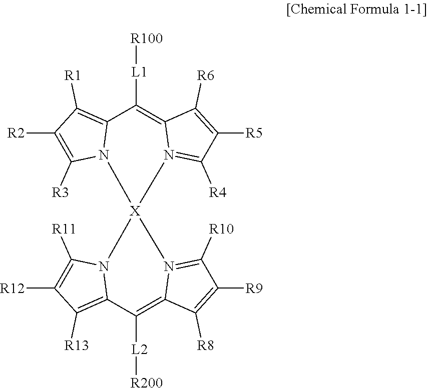
wherein, in the Chemical Formula 1-1,
R1, R2, R5, R6, R8, R9, R12 and R13 are the same as or different from each other, and each independently hydrogen; a halogen group; an aldehyde group; a nitrile group; a nitro group; a substituted or unsubstituted ester group; a substituted or unsubstituted amide group; a substituted or unsubstituted alkyl group; a substituted or unsubstituted cycloalkyl group; a substituted or unsubstituted aryl group; a substituted or unsubstituted aryloxy group; a substituted or unsubstituted arylthio group; or a substituted or unsubstituted heteroaryl group,
R3, R4, R10 and R11 are each independently hydrogen; a substituted or unsubstituted alkyl group; or a substituted or unsubstituted cycloalkyl group,
L1 and L2 are each independently O or S,
R100 and R200 are each independently a substituted or unsubstituted aryl group; or a substituted or unsubstituted heteroaryl group, and
X is Zn, Co, Ni or Pd.

13. The composition for forming an optical film of claim 12, wherein the Chemical Formula 1-1 is represented by any one of the following Chemical Formulae 1-1-1 to 1-1-8: 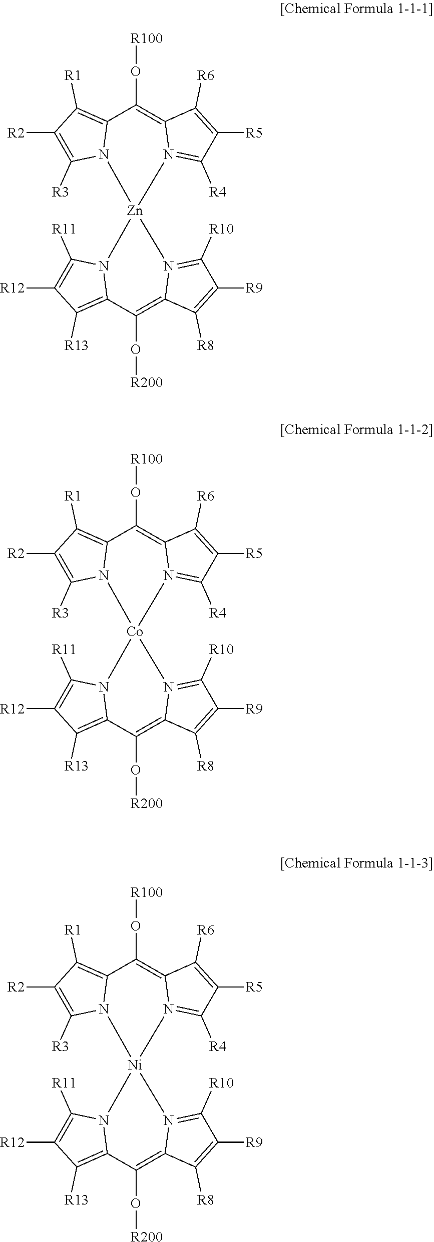 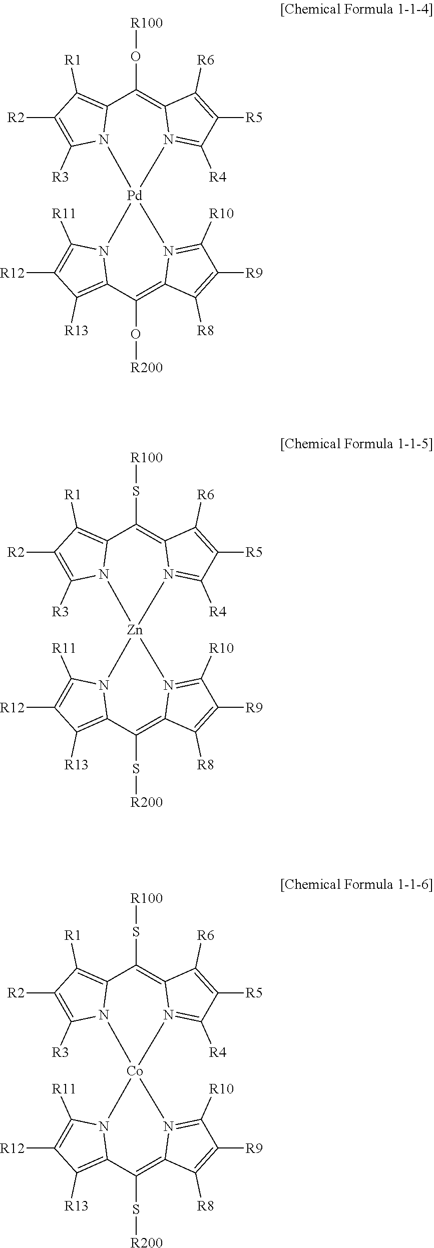 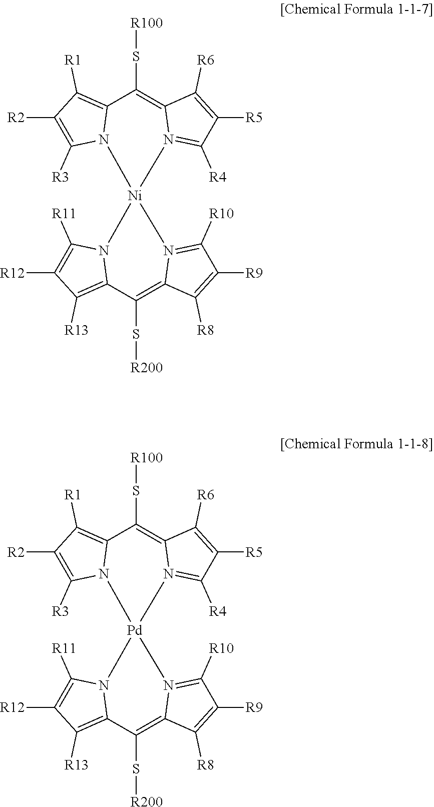
wherein, in the Chemical Formulae 1-1-1 to 1-1-8,
each substituent has the same definition as in Chemical Formula 1-1.

14. The composition for forming an optical film of claim 1, wherein the Chemical Formula 1 is represented by the following Chemical Formula 2-1: 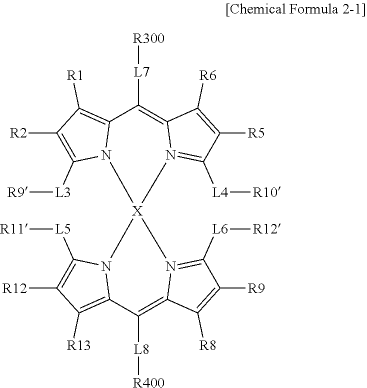
wherein, in the Chemical Formula 2-1,
R1, R2, R5, R6, R8, R9, R12 and R13 are the same as or different from each other, and each independently hydrogen; a halogen group; an aldehyde group; a nitrile group; a nitro group; a substituted or unsubstituted ester group; a substituted or unsubstituted amide group; a substituted or unsubstituted alkyl group; a substituted or unsubstituted cycloalkyl group; a substituted or unsubstituted aryl group; a substituted or unsubstituted aryloxy group; a substituted or unsubstituted arylthio group; or a substituted or unsubstituted heteroaryl group,
R9′ to R12′ are the same as or different from each other, and each independently a substituted or unsubstituted alkyl group; a substituted or unsubstituted aryl group; or a substituted or unsubstituted heteroaryl group,
L3 to L6, L7 and L8 are each independently O or S,
R300 and R400 are each independently a substituted or unsubstituted aryl group; or a substituted or unsubstituted heteroaryl group, and
X is Zn, Co, Ni or Pd.

15. The composition for forming an optical film of claim 14, wherein the Chemical Formula 2-1 is represented by any one of the following Chemical Formulae 2-1-1 to 2-1-8: 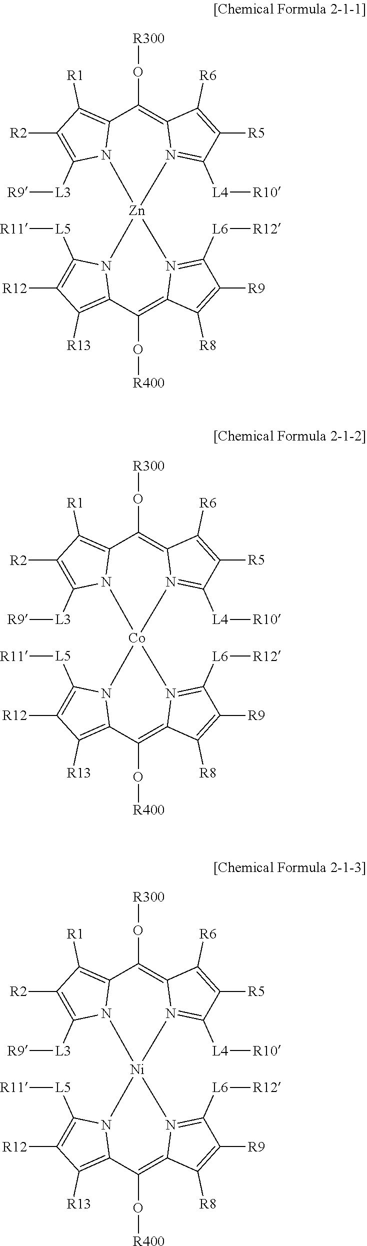 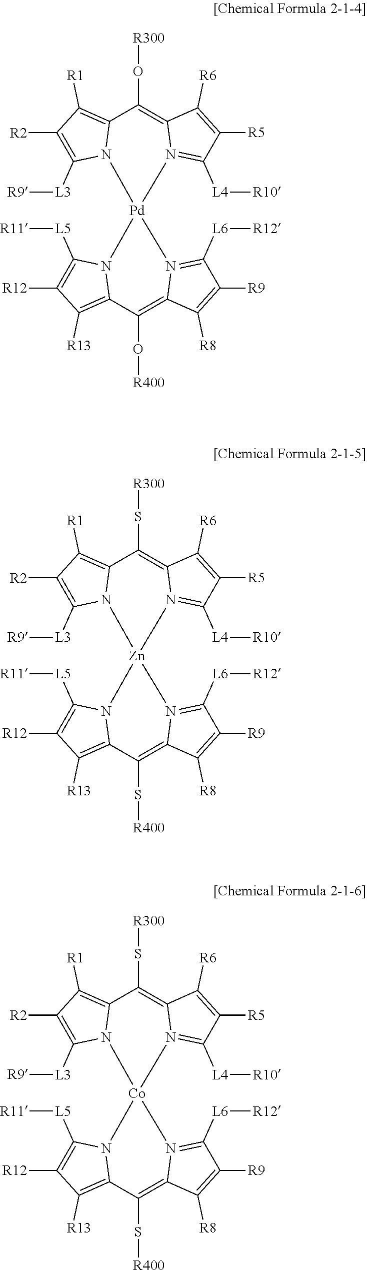 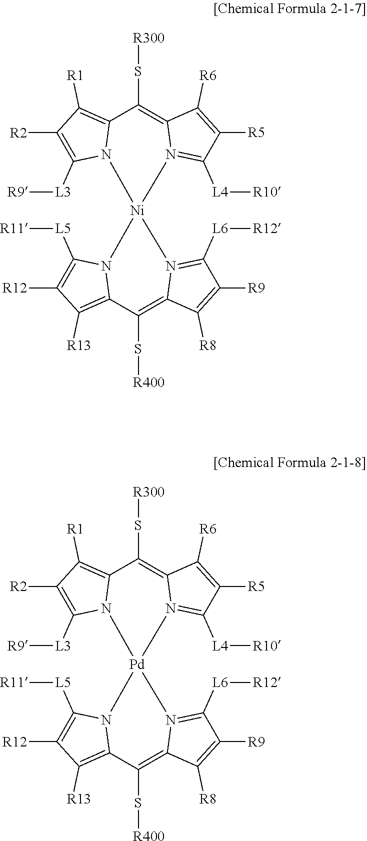
wherein, in Chemical Formulae 2-1-1 to 2-1-8,
each substituent has the same definition as in Chemical Formula 2-1.

16. The composition for forming an optical film of claim 1, wherein the Chemical Formula 1 is any one selected from the following compounds: 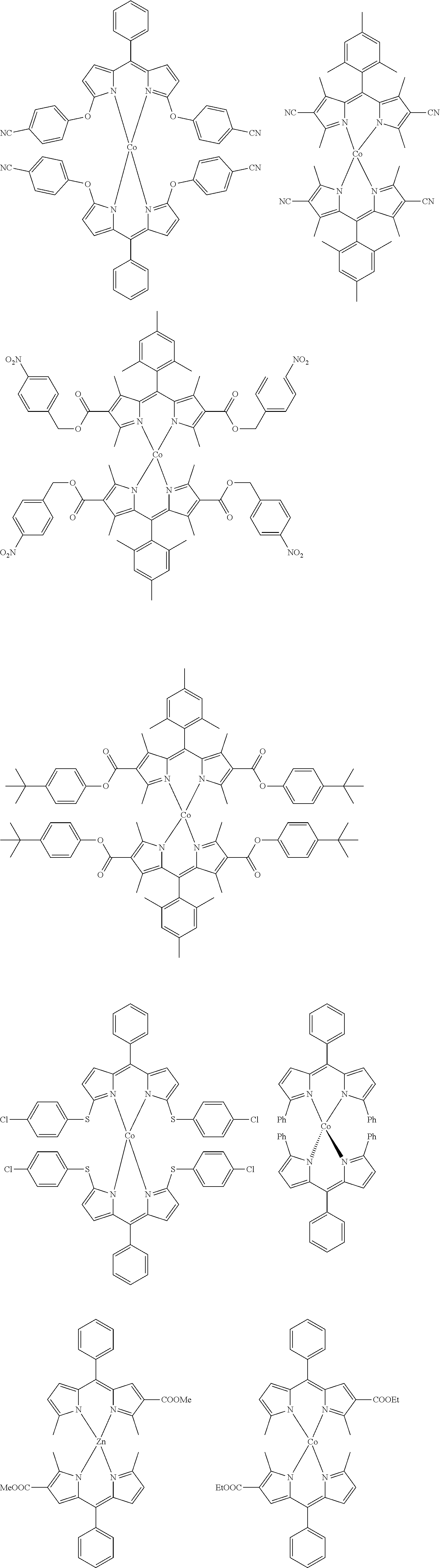 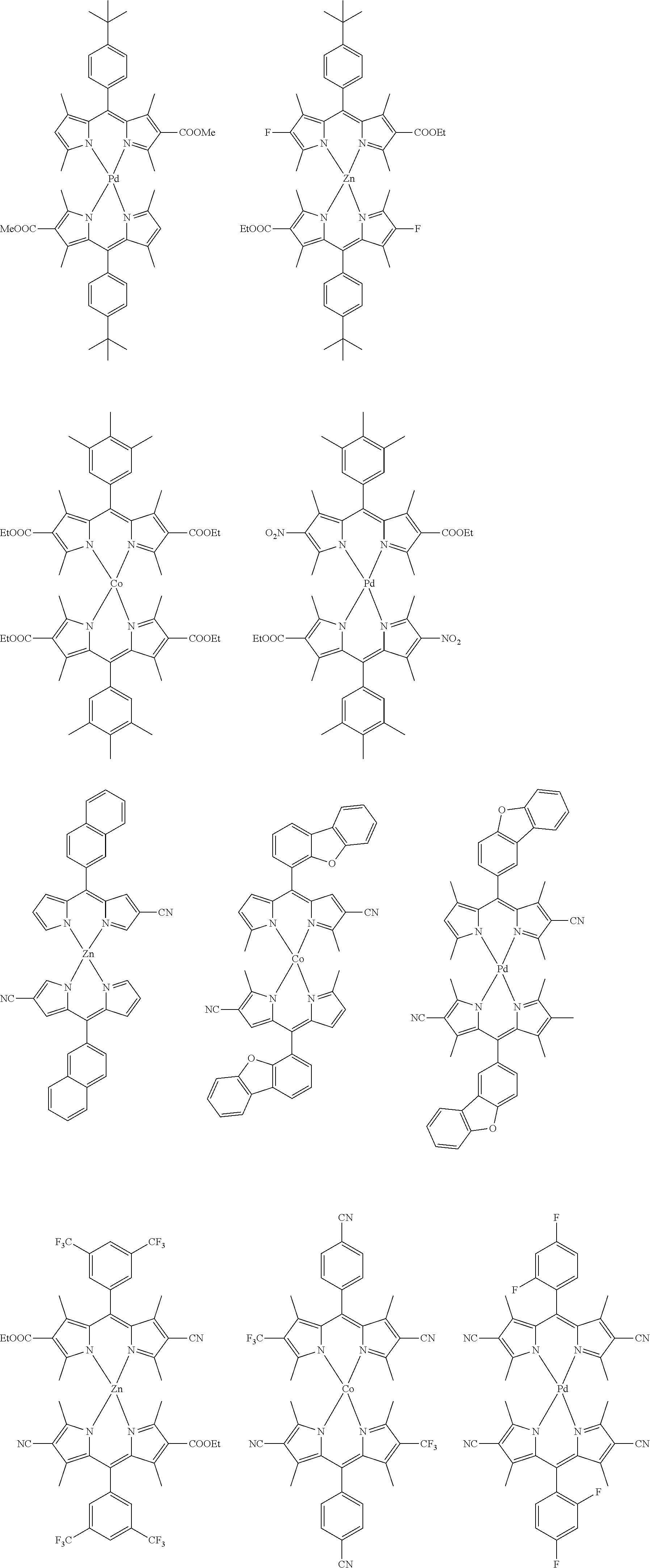 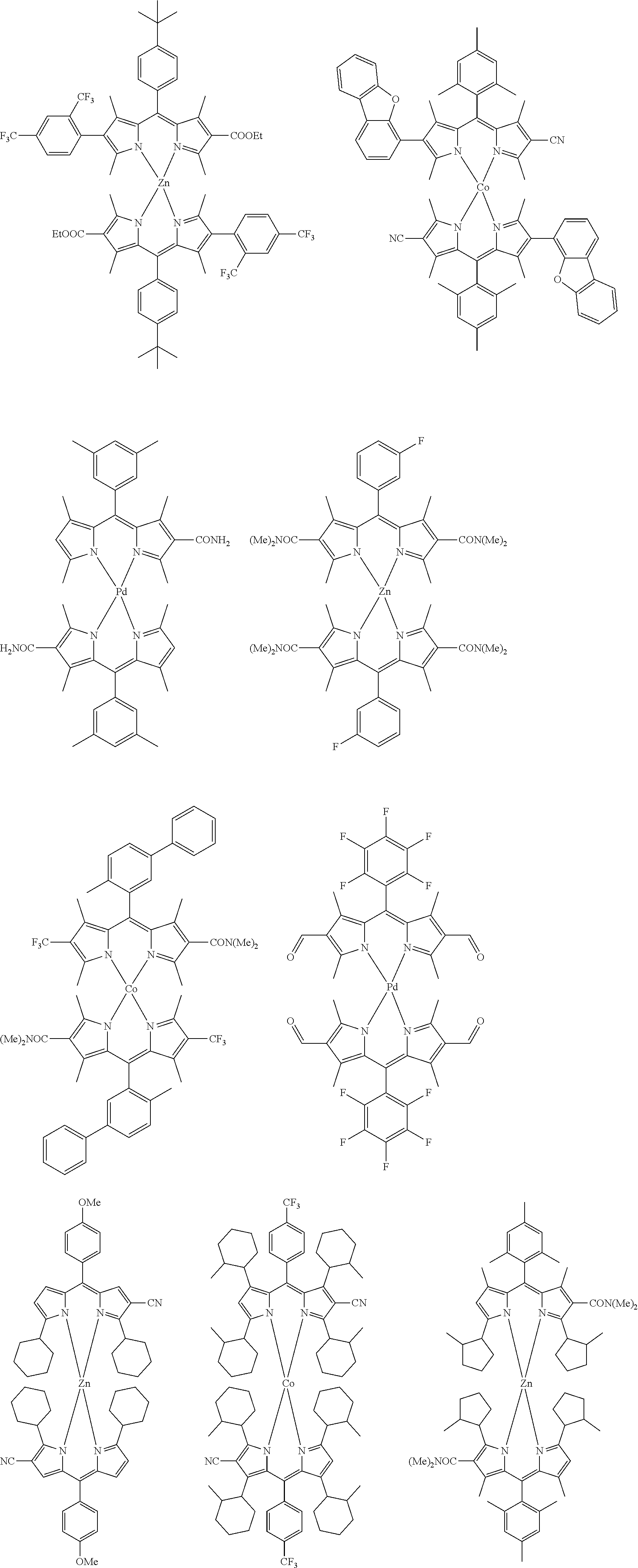 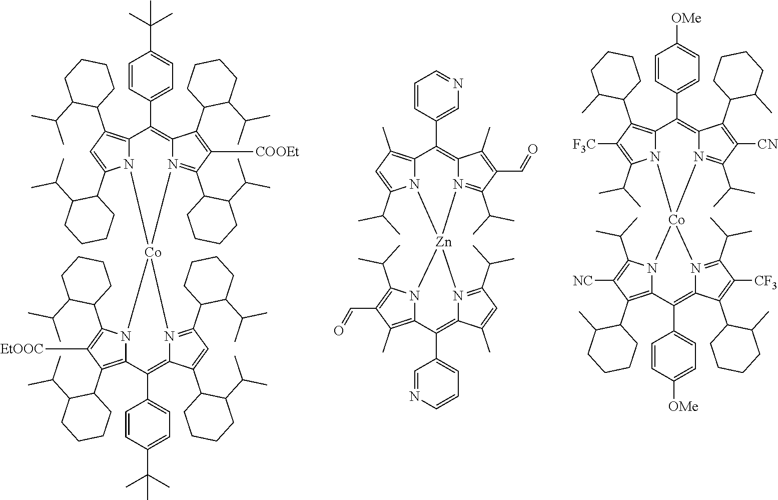 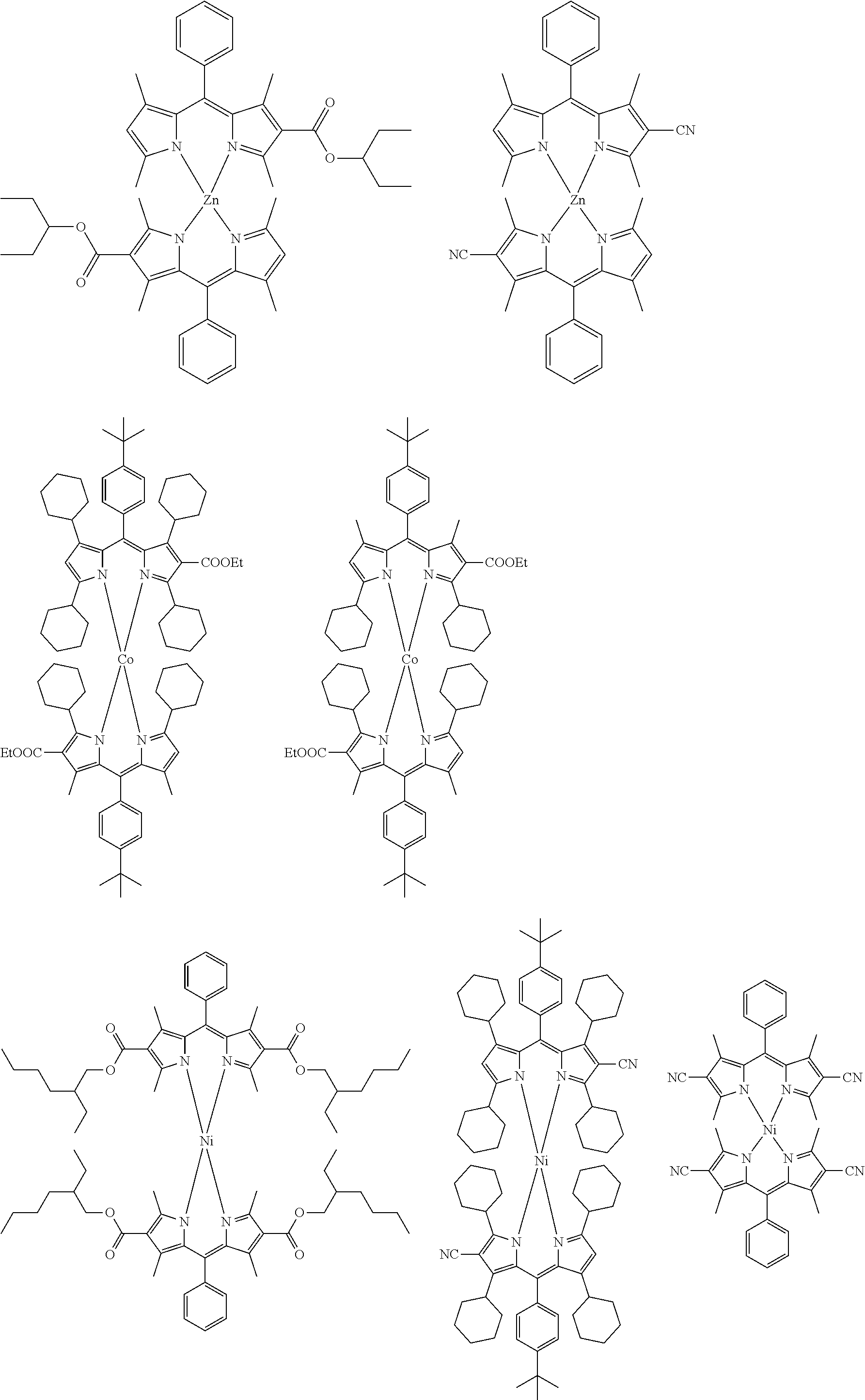 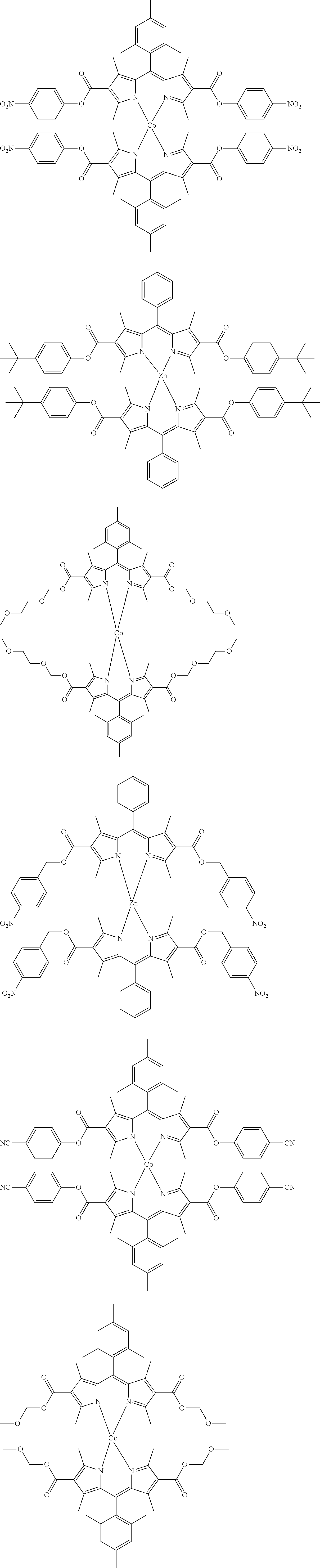 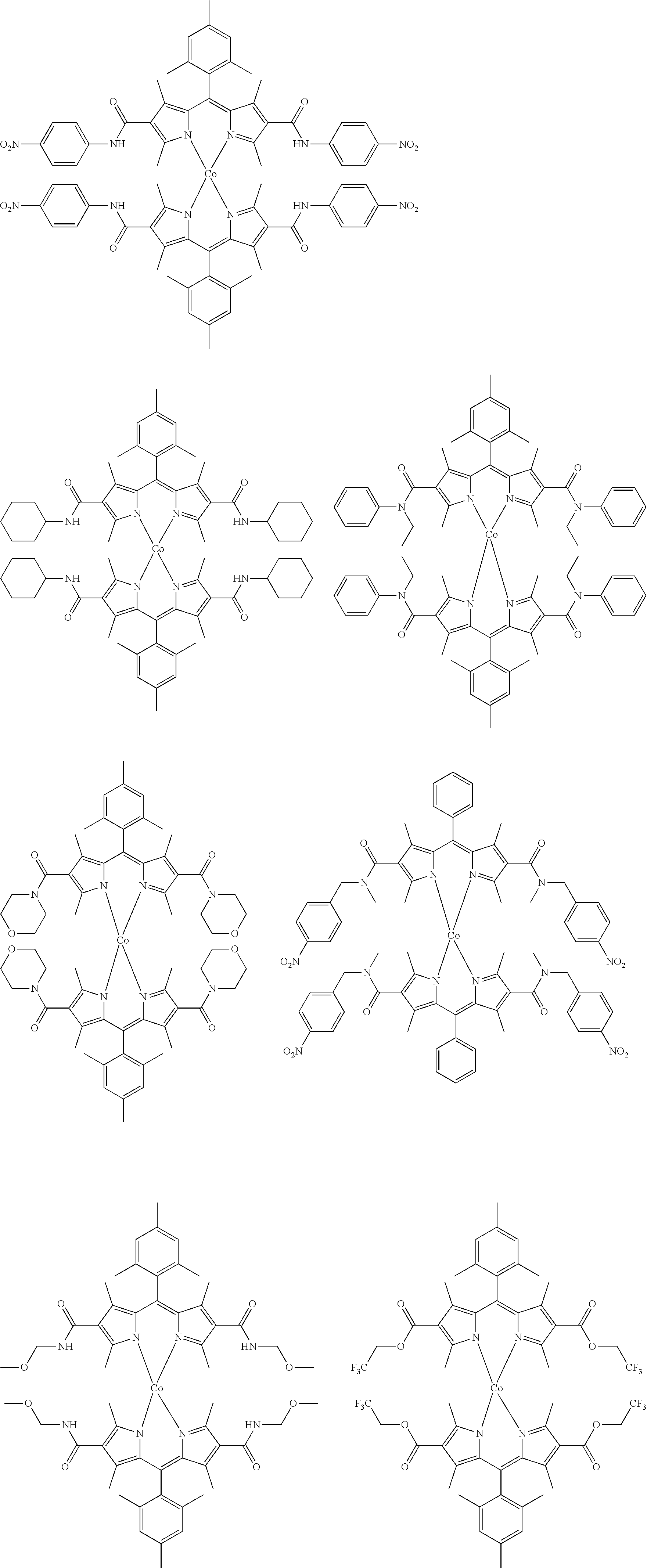 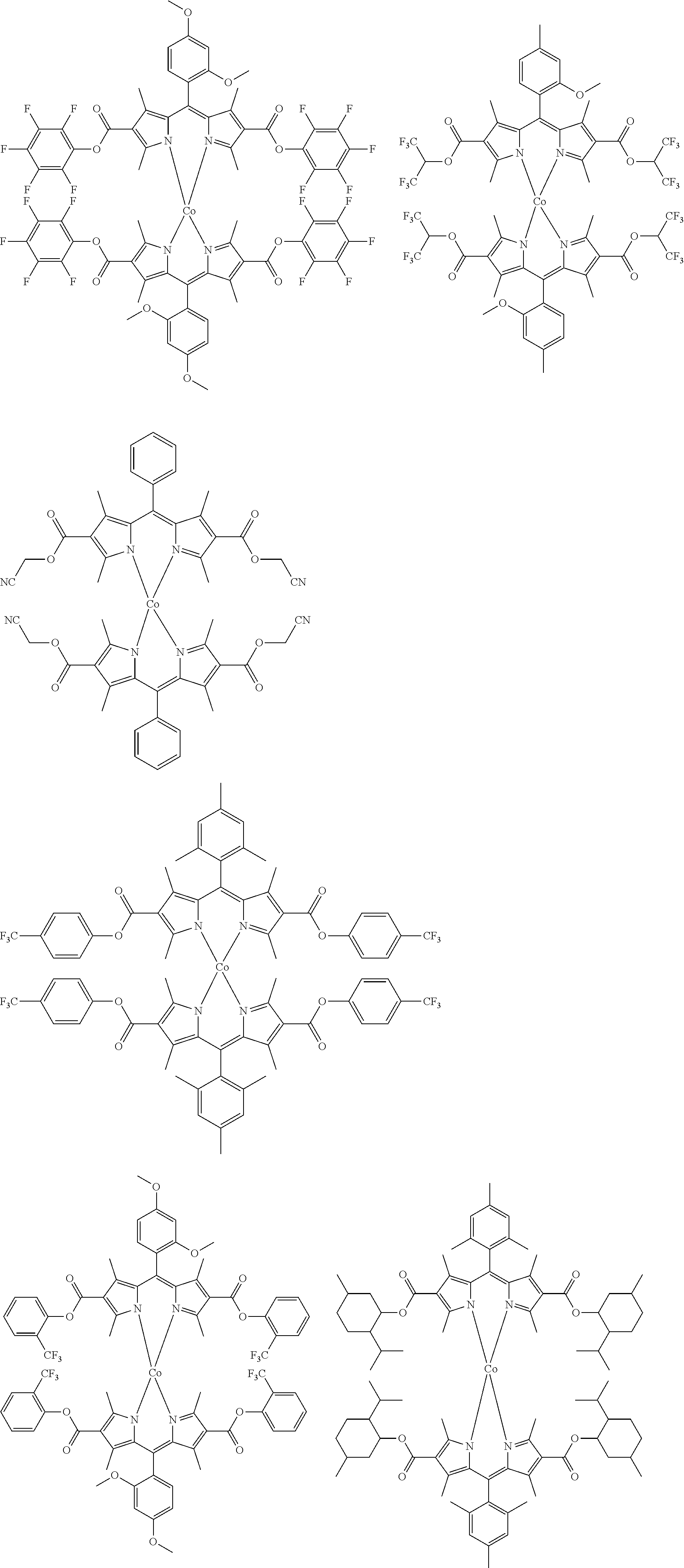 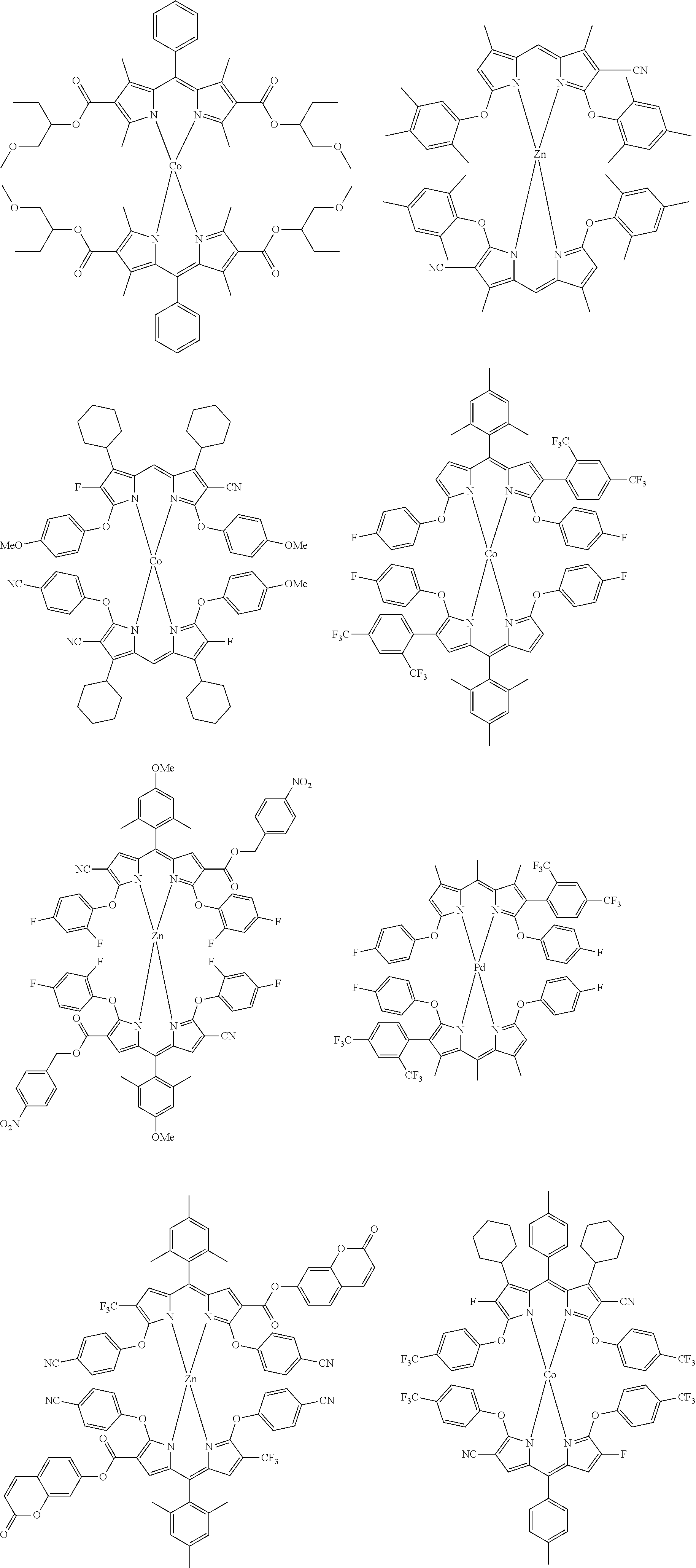 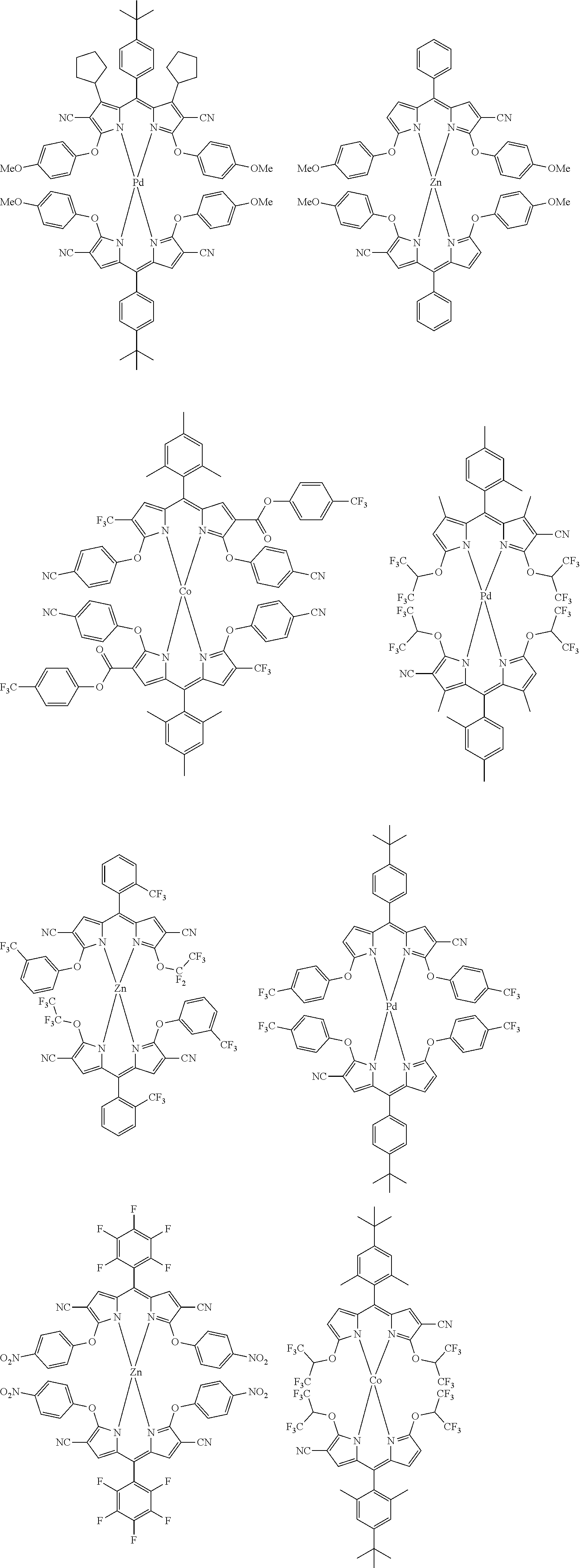 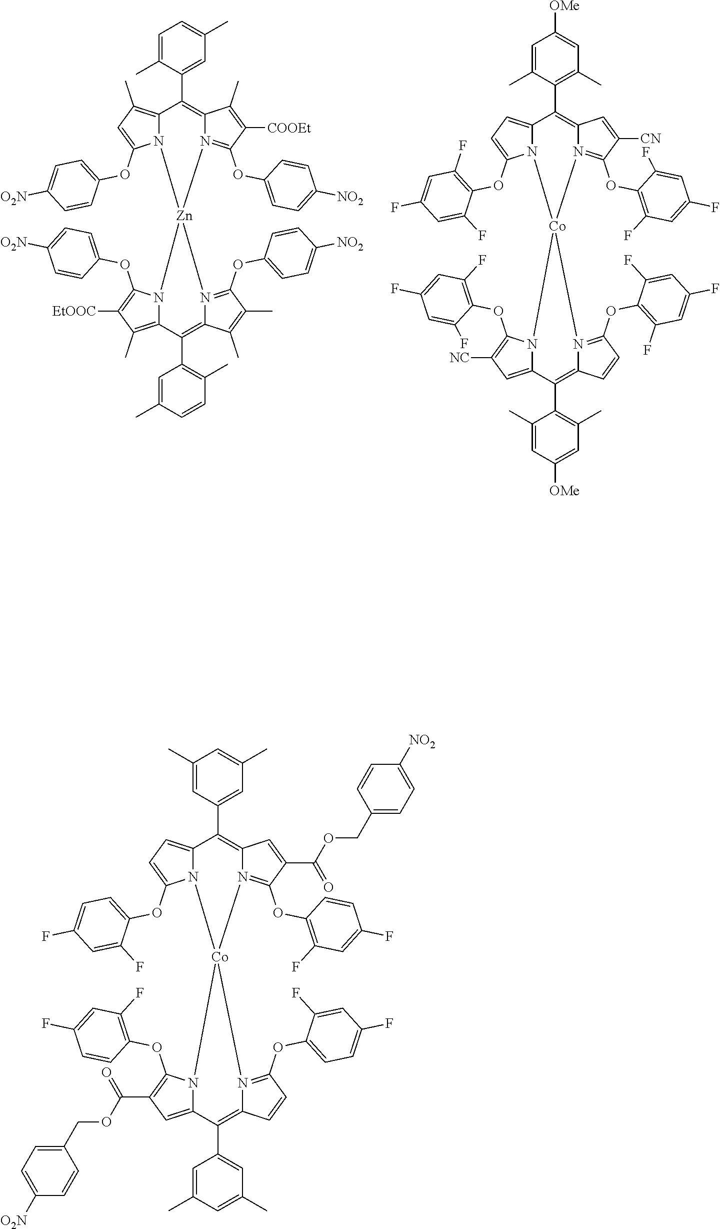 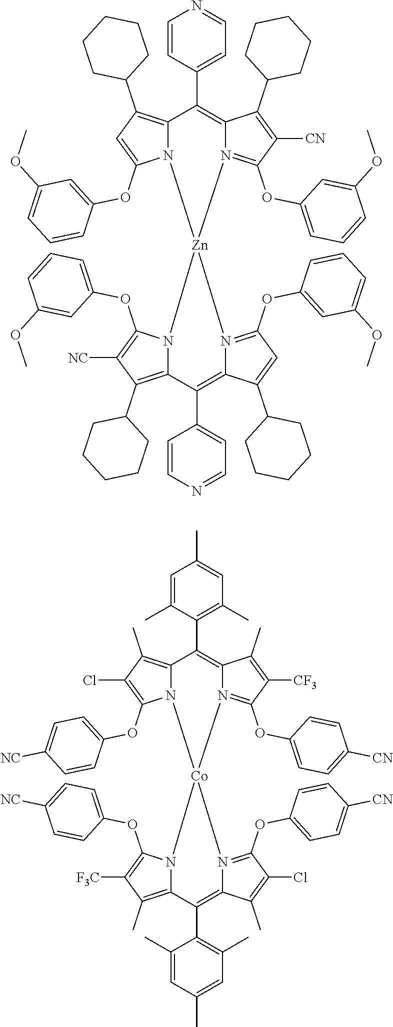 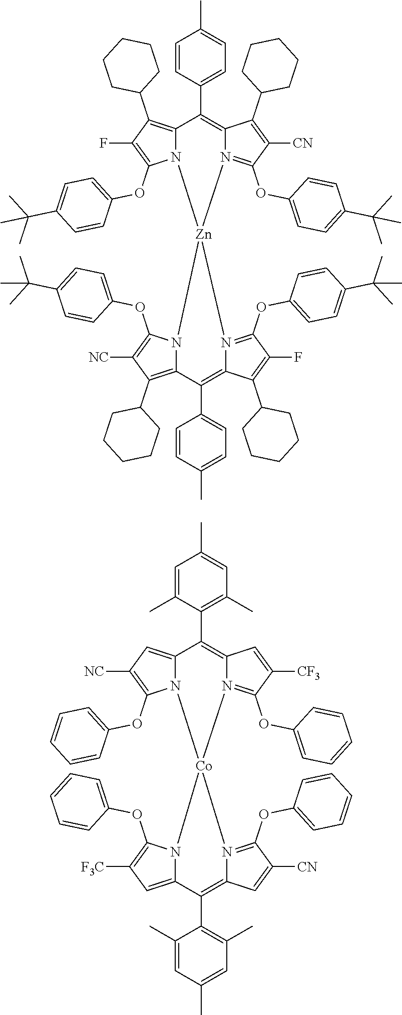 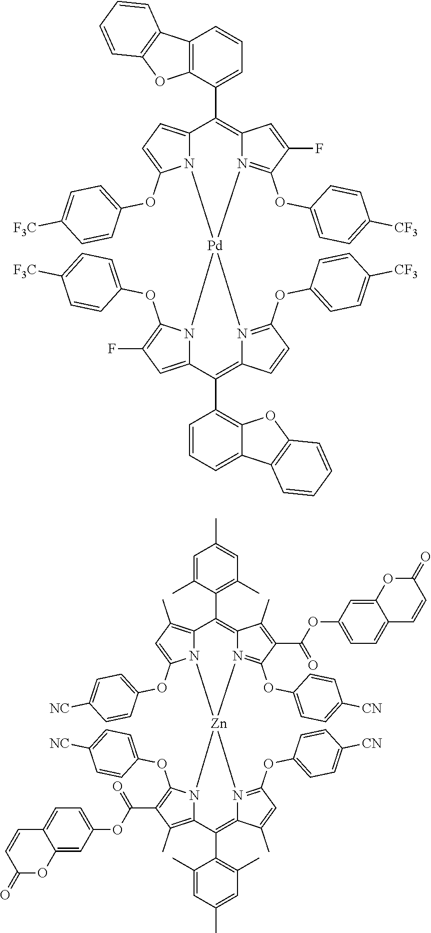 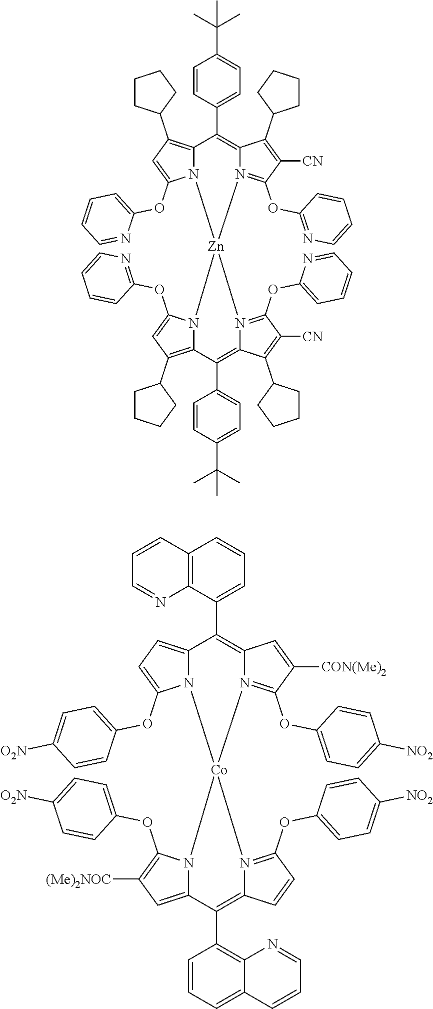 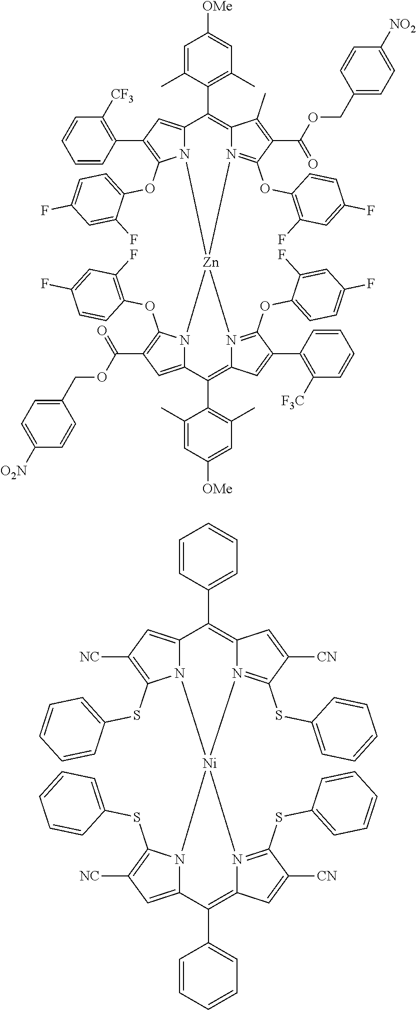 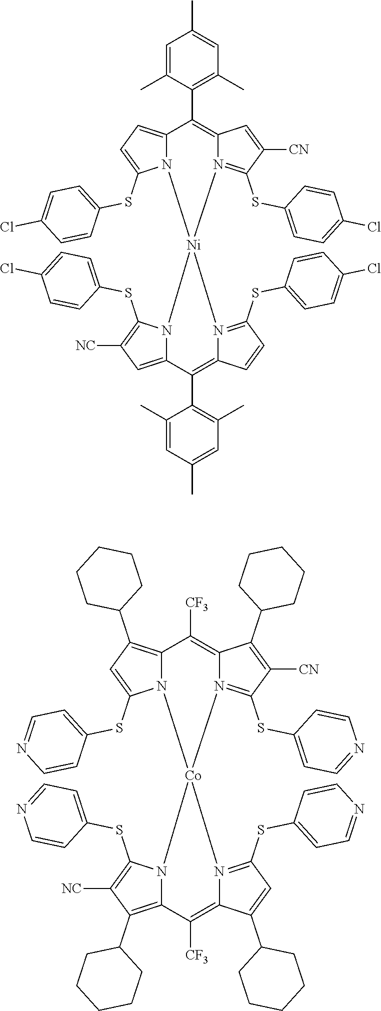 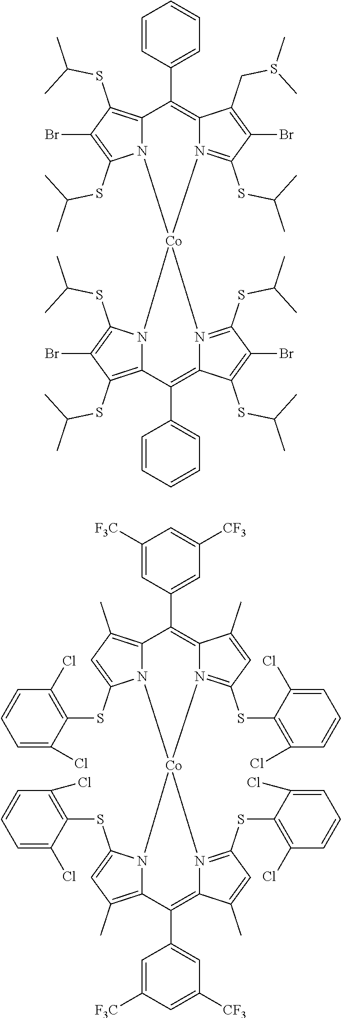 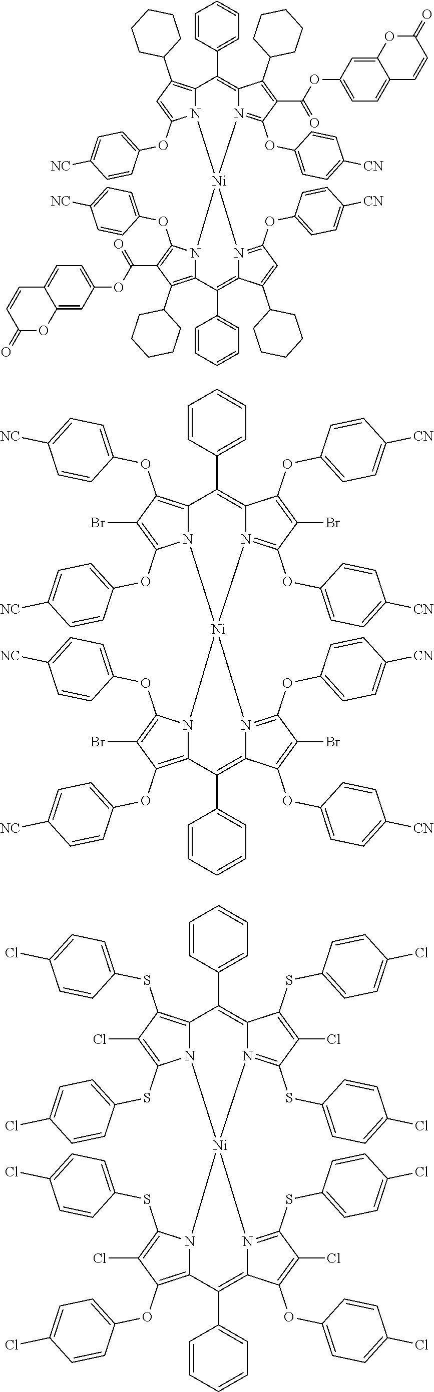 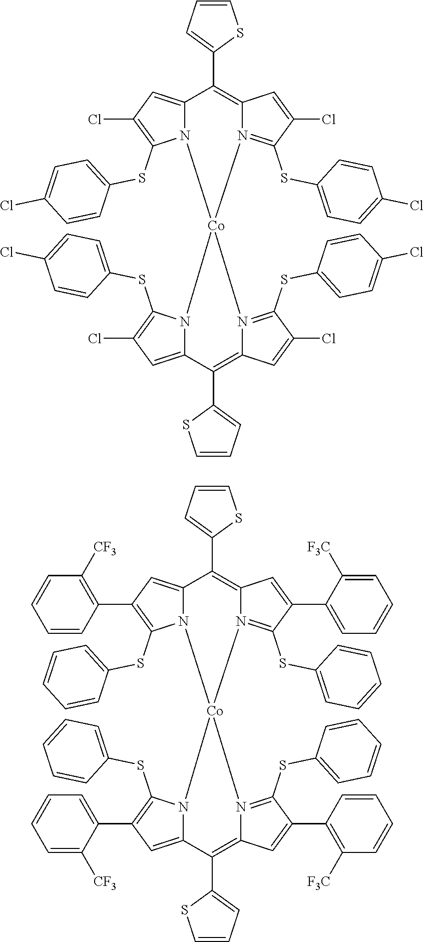 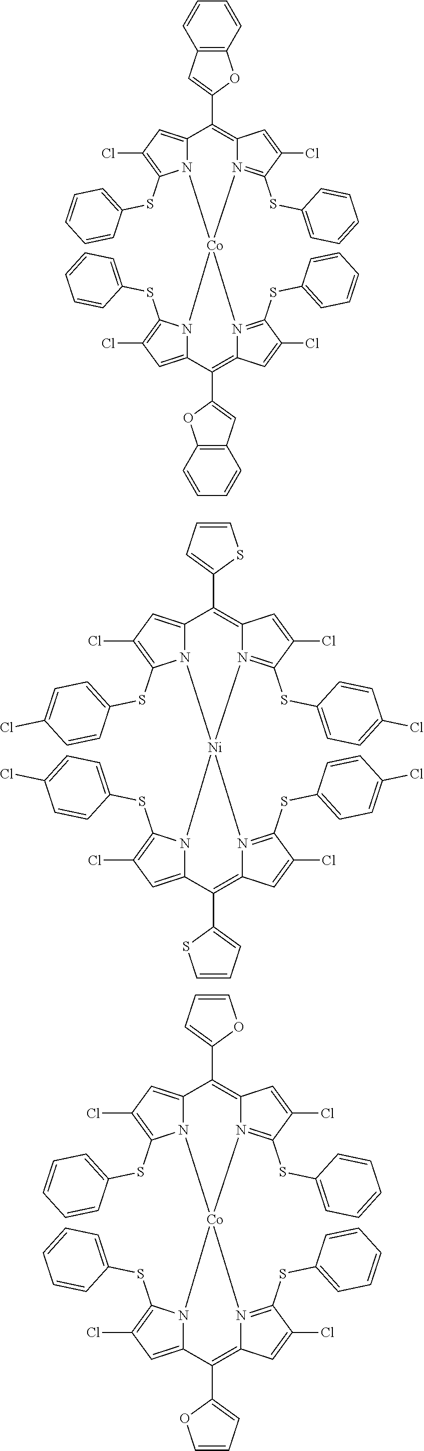 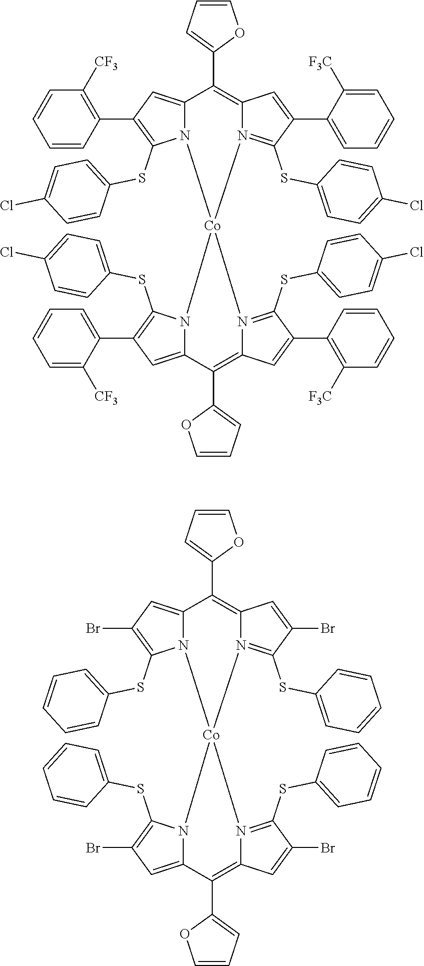 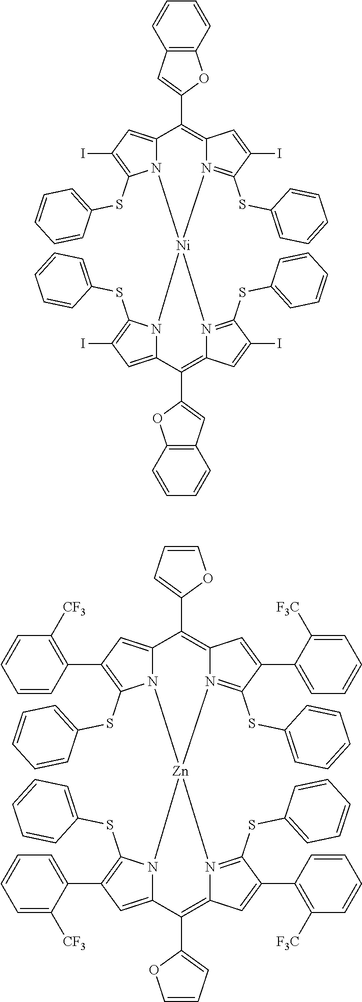 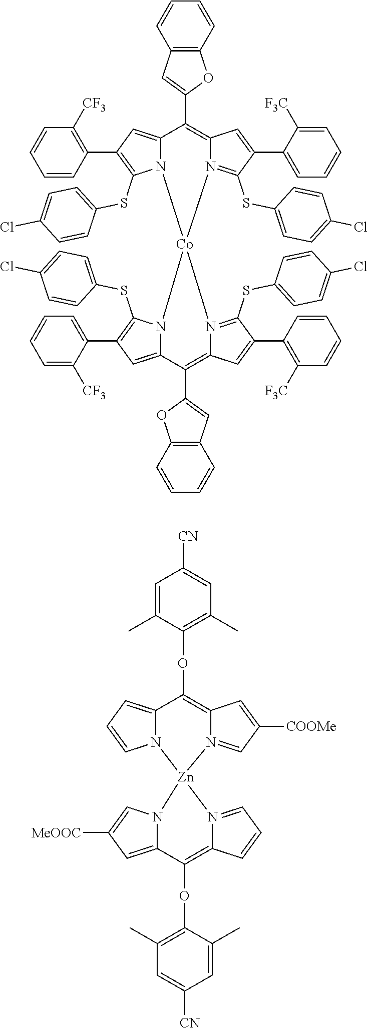 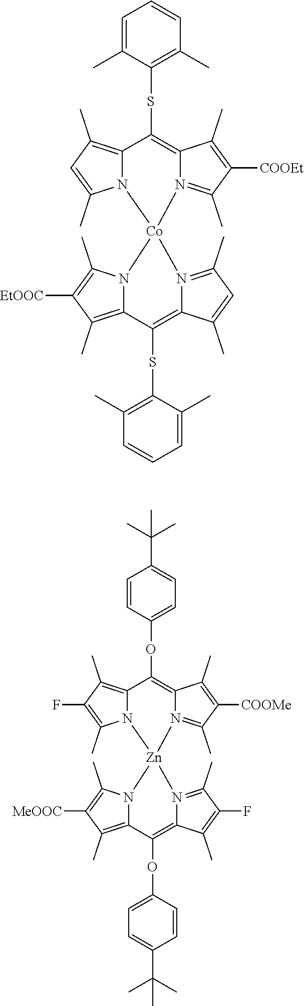 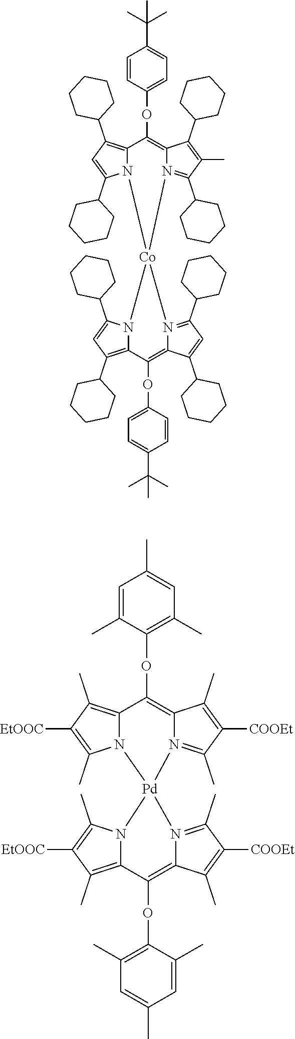 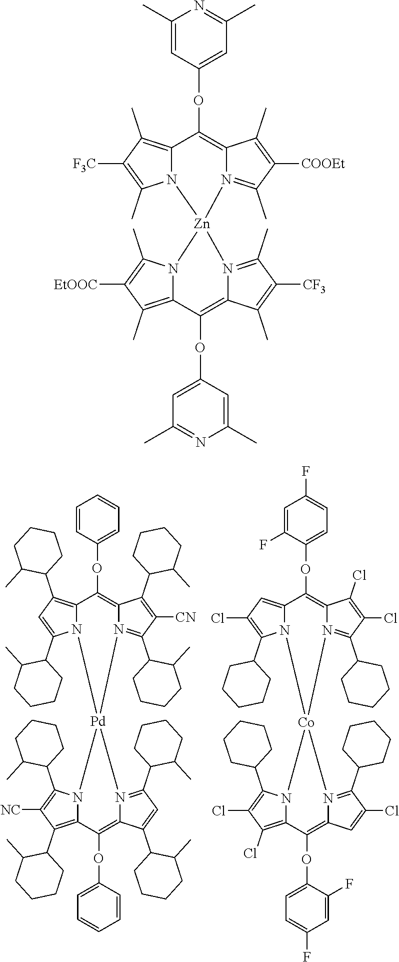 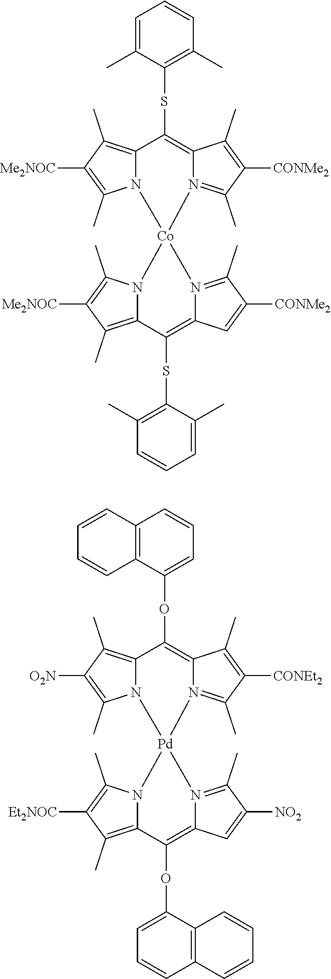 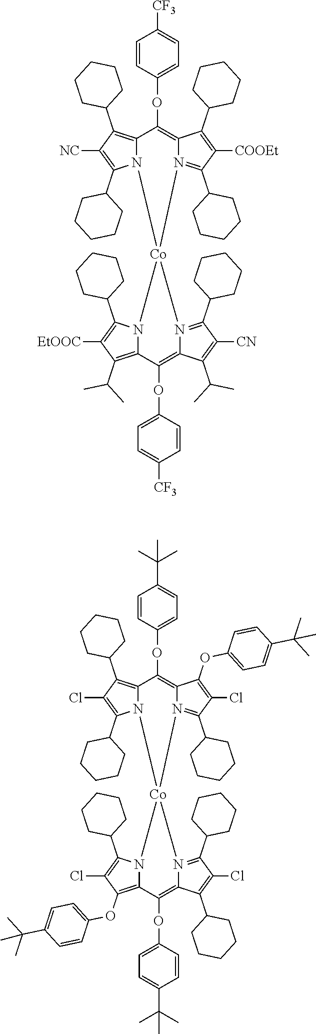 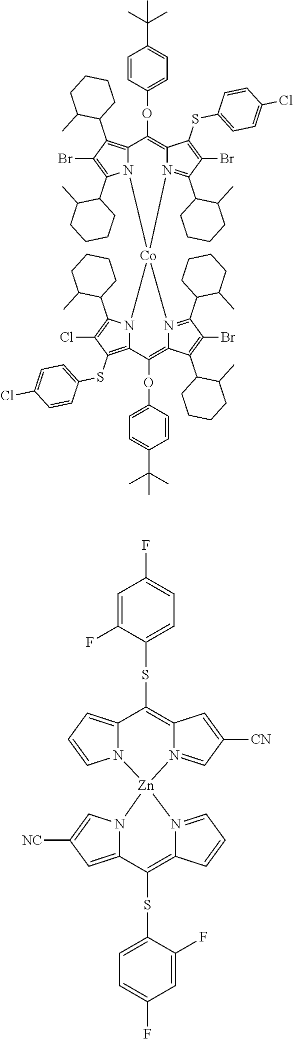 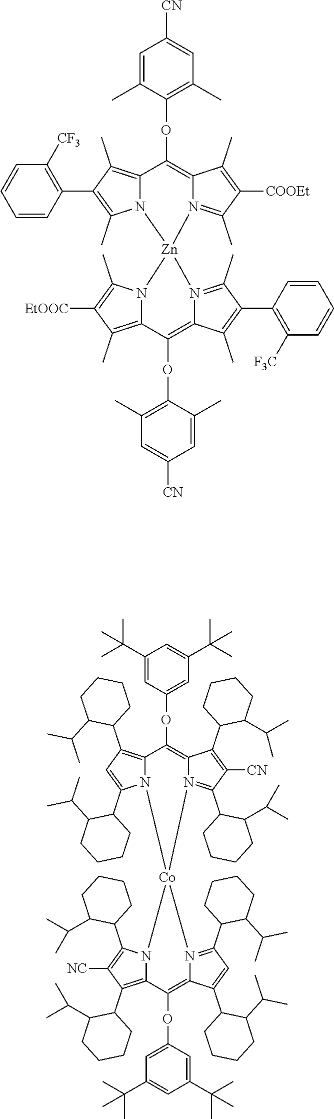 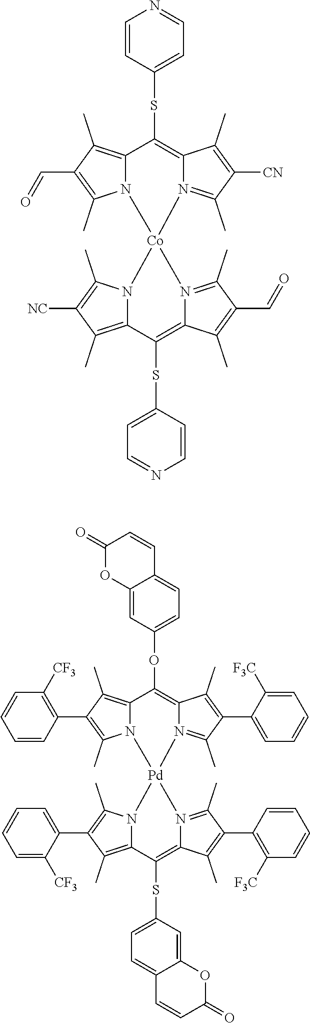 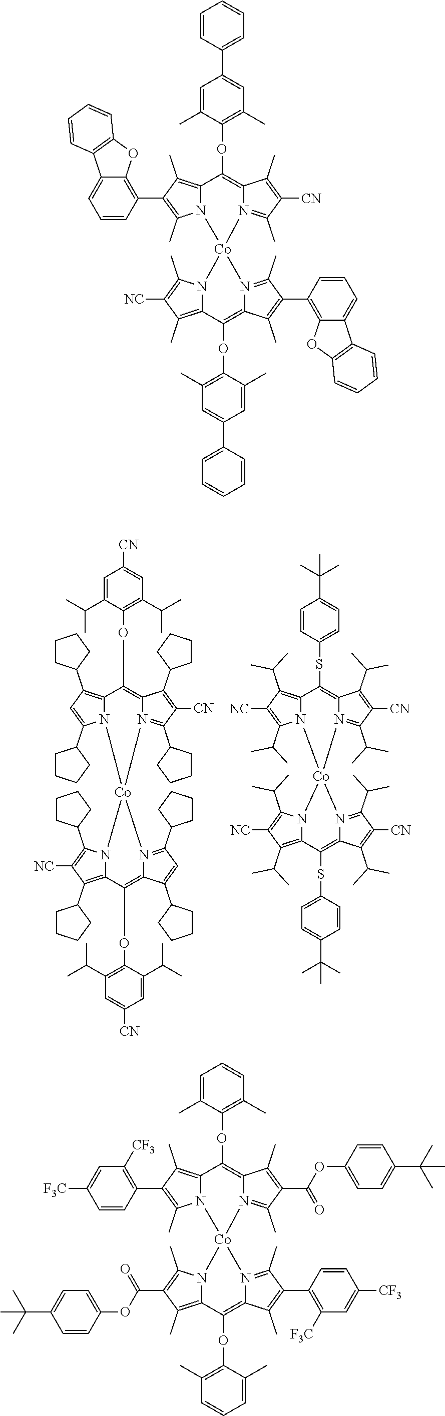 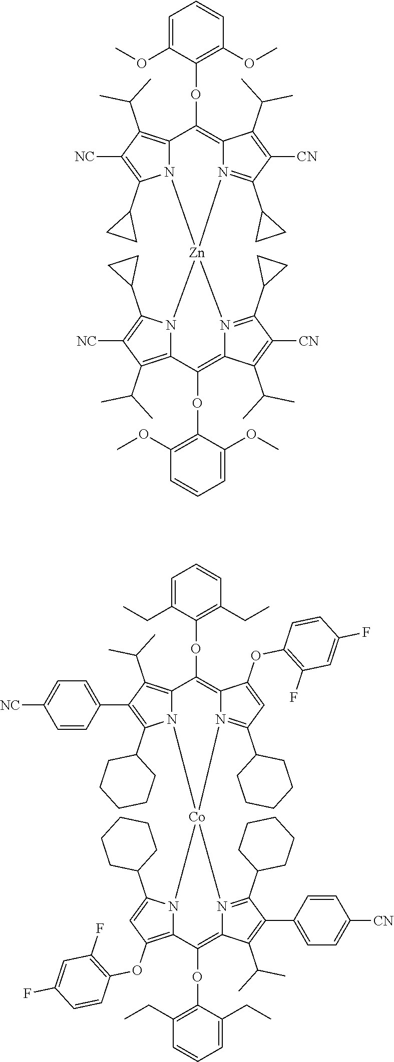 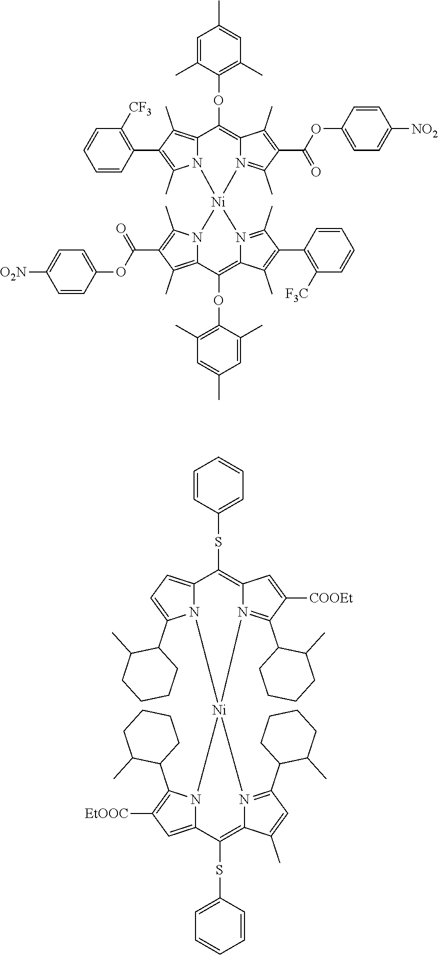 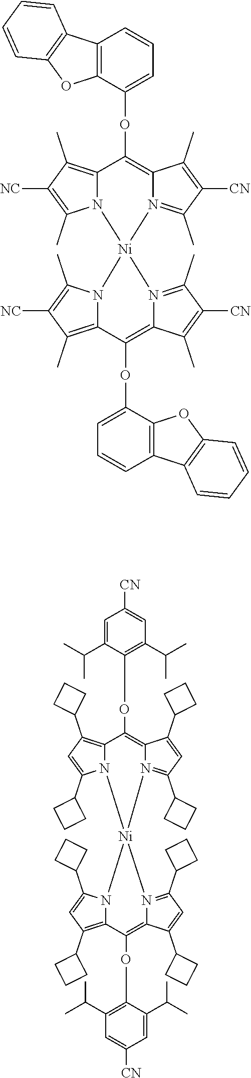 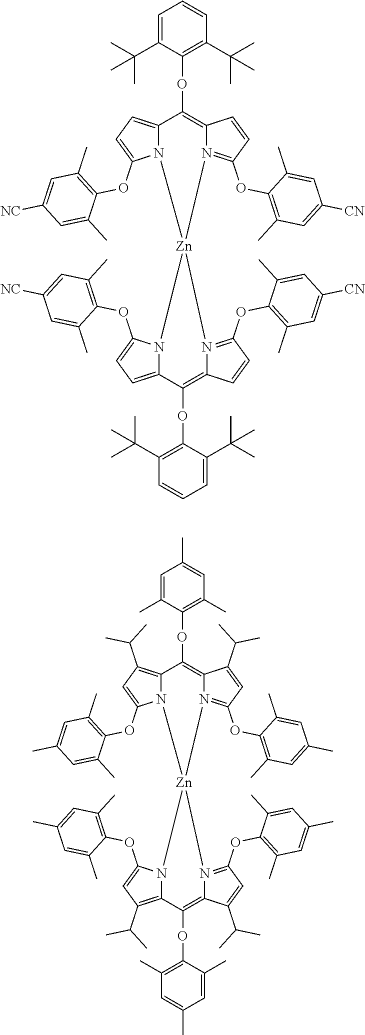 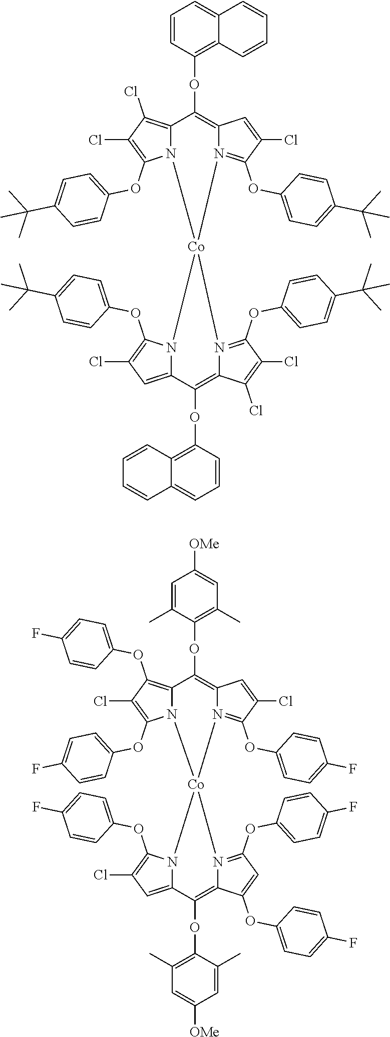 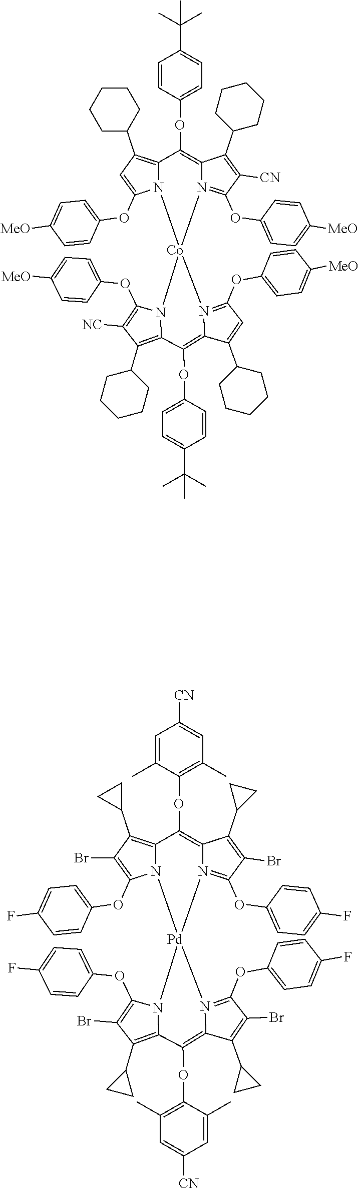 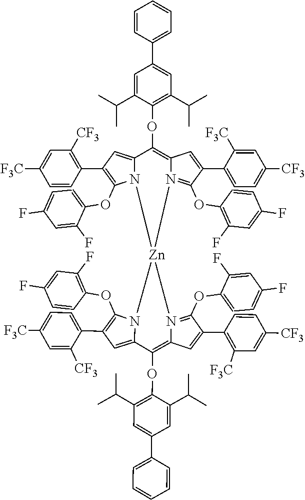 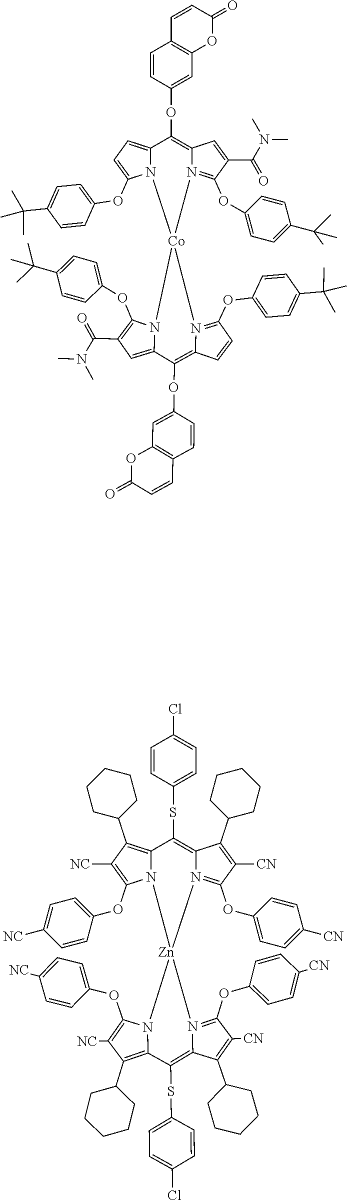 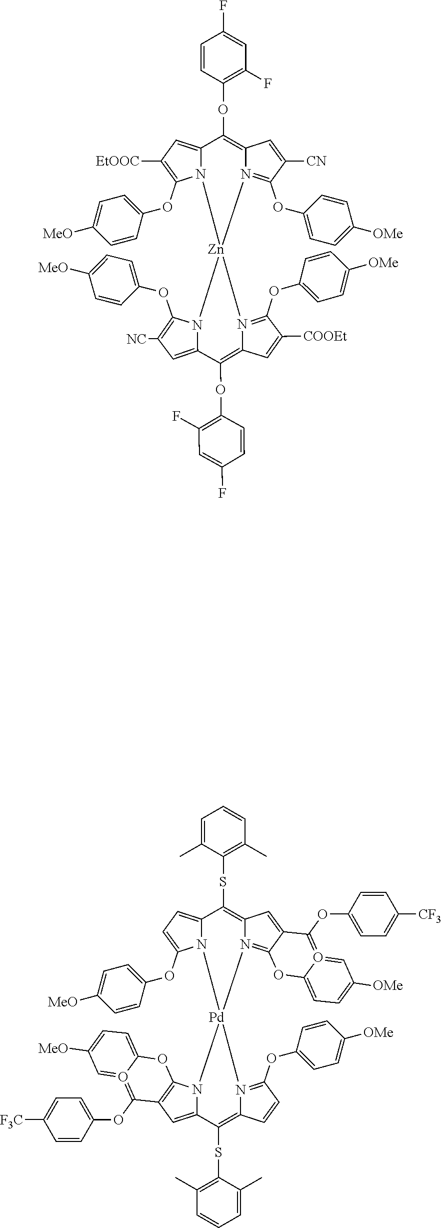 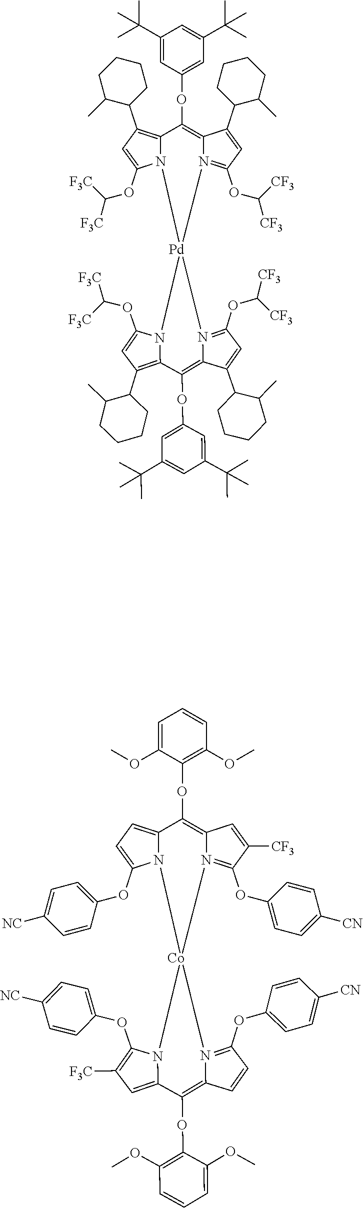 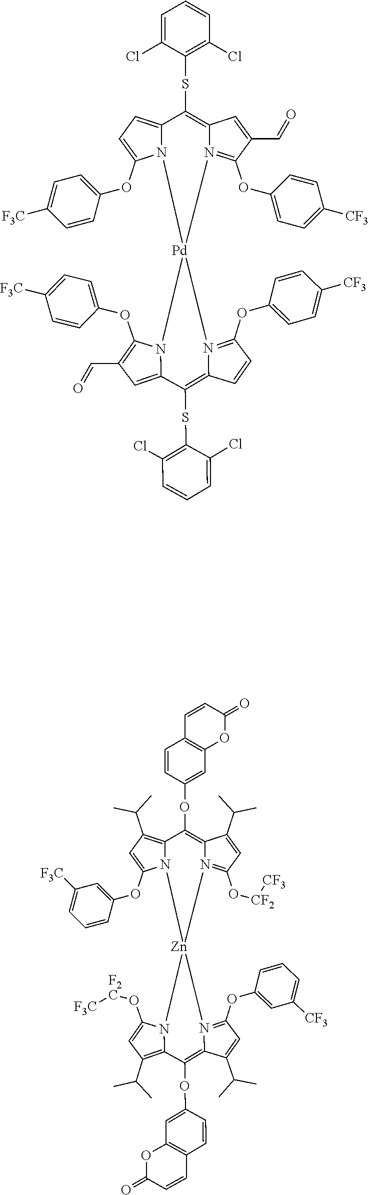 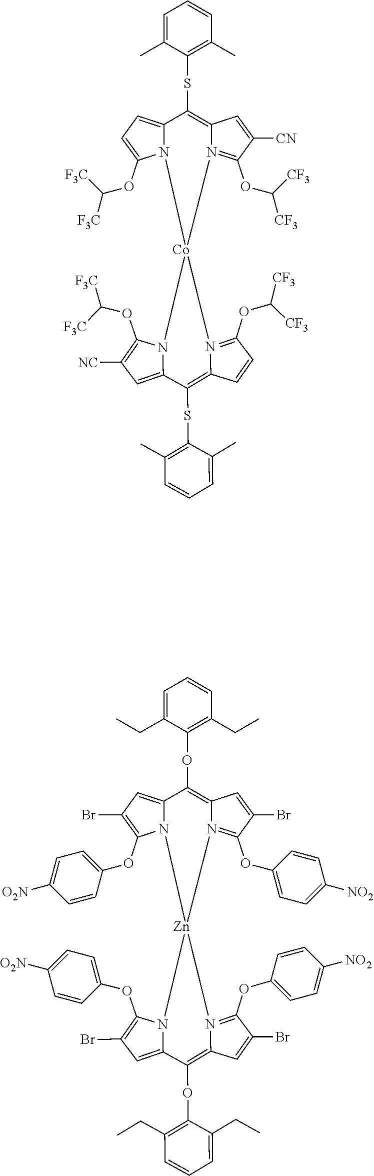 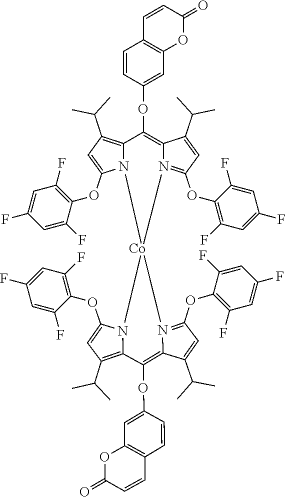 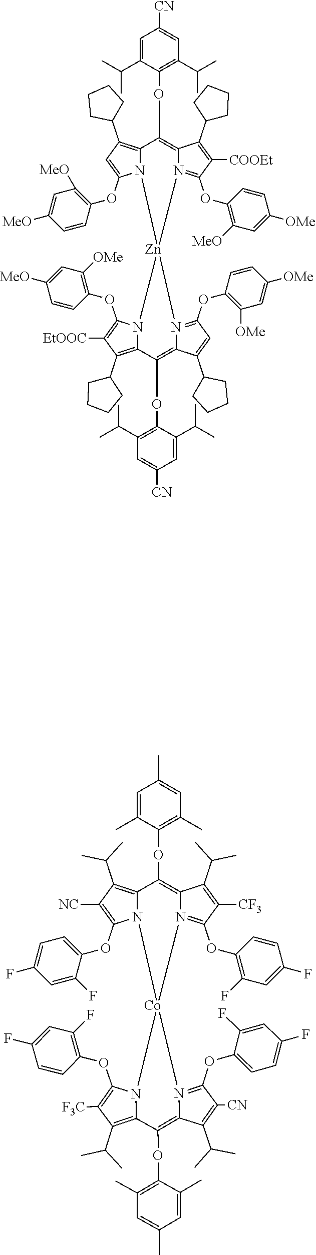 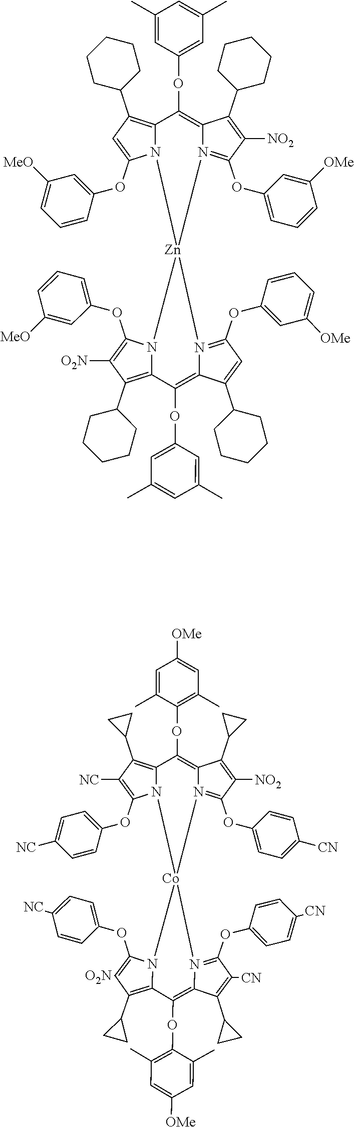 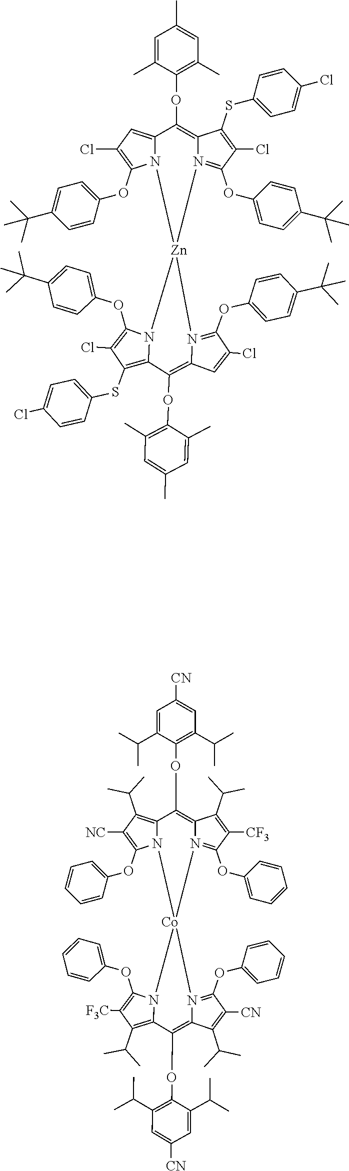 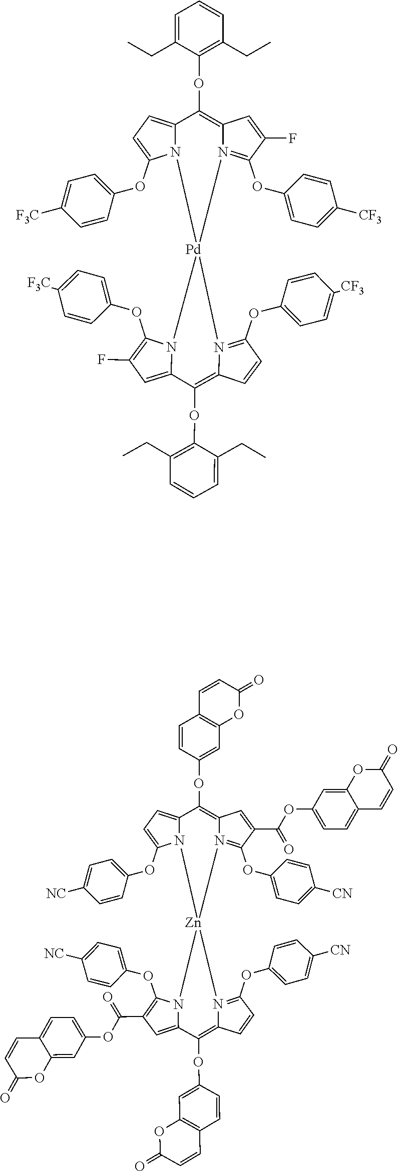 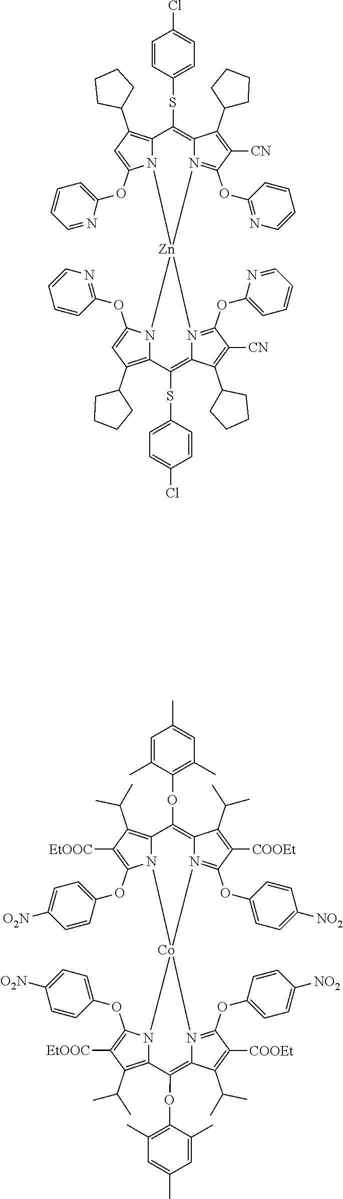 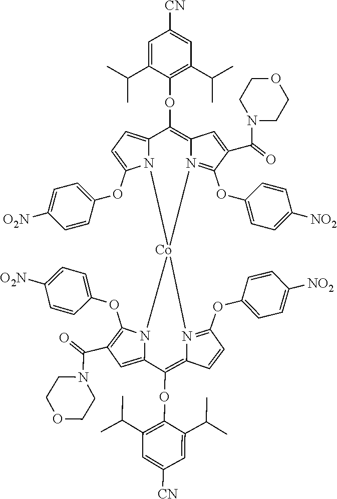 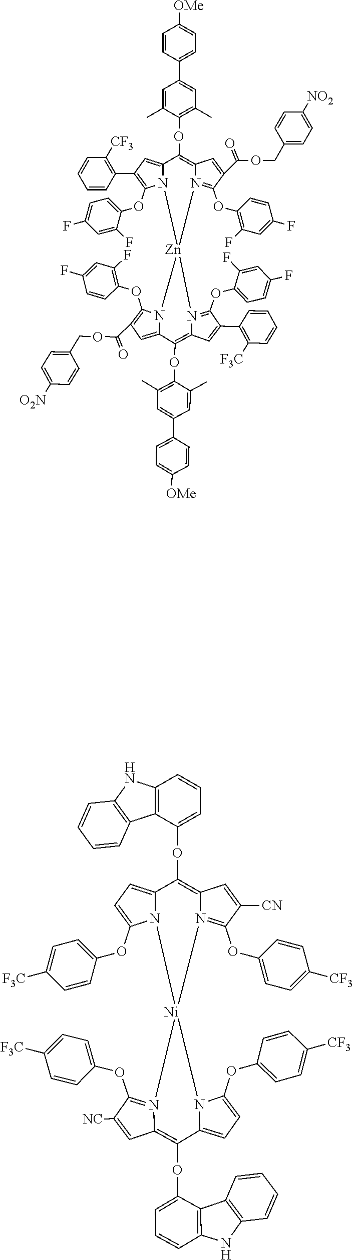 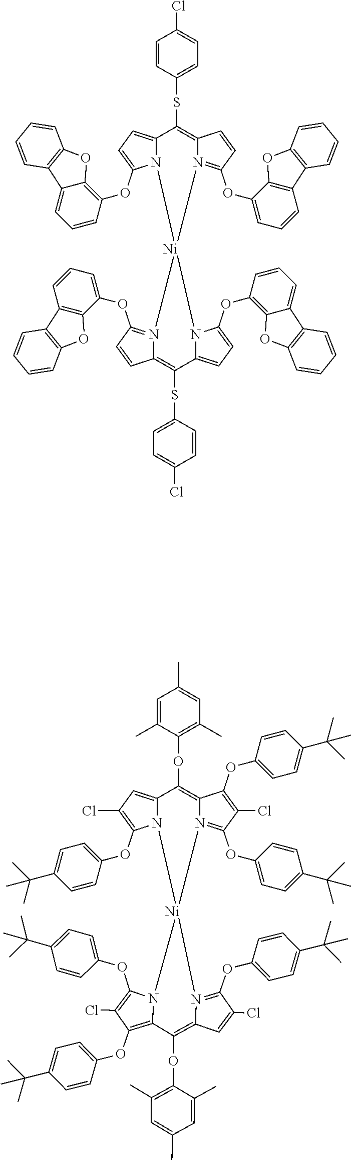 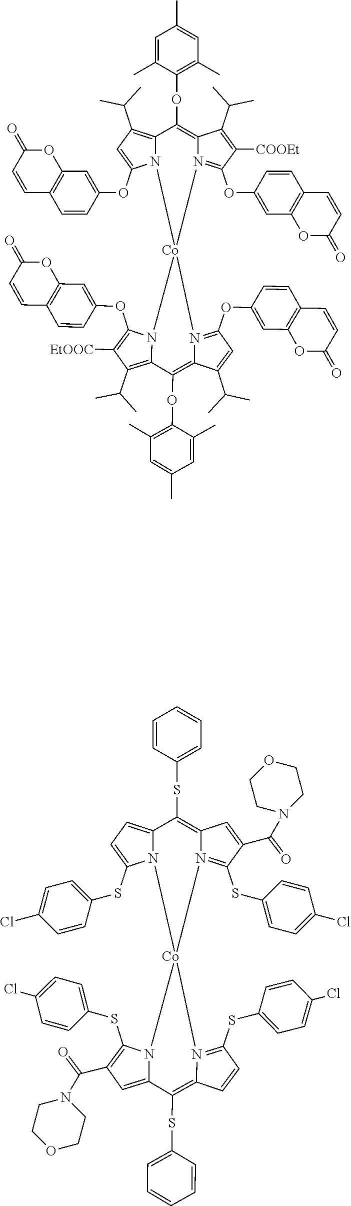 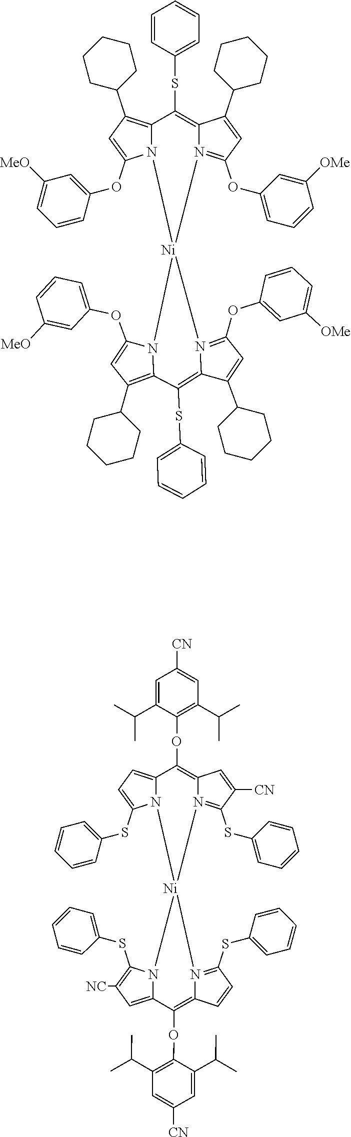 [chemical structure]
